SEQUENCE LISTING (51 sequences)

SEQ ID NO: 1 - protein, 85 aa, synthetic construct
IDQLVQETLAGKYGNGEQRKAALGSQYQAVMAVINGKATAPKKTVDQLAQEVIQGKHGNGEDRKKSLGPDYDAVQKRVTEILKGS

SEQ ID NO: 2 - protein, 84 aa, synthetic construct
SRSYRETGTMTVTVDALNVRRAPNTSGEIVAVYKRGESFDYDTVIIDVNGYVWVSYIGGSGKRNYVATGATKDGKRFGNAWGTF

SEQ ID NO: 3 - protein, 261 aa, synthetic construct
GKHLVICGHGQGRTGYDPGAVNAKLGITEAGKVRELSKLMSKYSGQQIDFITEQNVYDYRSITSIGKGYDSITELHFNAFNGSAKGTEVLIQSSLEADKEDMAILSLLSRYFQNRGIKKVDWLYNANQAASRGYTYRLVEIAFIDNEQDMAIFENKKEDIAKGLVSAITGVEVKTIVPSPPSSTVGSSGTPSKSIYLVGDSLRVLPHATHYQTGQKIANWVKGRTYKILQVKNVHQSNSKRAYLLDGIKSWVLEQDVEGTT

SEQ ID NO: 4 - protein, 145 aa, synthetic construct
TTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYIT

SEQ ID NO: 5 - DNA, 255 bp, synthetic construct
  source 1..255: mol_type = other DNA
atcgatcagctggtacaggaaaccttagctggcaagtatggcaatggcgagcaacgcaaagcggccctgggtagtcagtaccaagcagtgatggcggtgattaacggtaaggcgacagccccgaagaaaaccgtcgatcagttagcgcaagaggtcattcaaggcaagcacggtaatggcgaggatcgcaagaagtcgctgggccccgattacgatgcggtacagaagcgcgtcaccgagattttgaagggctcg

SEQ ID NO: 6 - DNA, 252 bp, synthetic construct
  source 1..252: mol_type = other DNA
agccgtagctatcgtgaaaccggcaccatgaccgttaccgttgatgcactgaatgttcgtcgtgcaccgaataccagcggtgaaattgttgcagtgtataaacgtggtgagagcttcgattatgataccgtgattattgatgtgaacggttatgtttgggtgagctatattggtggttcaggcaaacgtaattatgttgcaaccggtgccaccaaagatggtaaacgctttggtaatgcatggggcaccttt

SEQ ID NO: 7 - DNA, 783 bp, synthetic construct
  source 1..783: mol_type = other DNA
ggcaaacatctggtgatttgtggtcatggtcagggtcgtaccggttatgatccgggtgcagttaatgcaaaactgggtattaccgaagcaggtaaagttcgtgaactgagcaaactgatgagcaaatatagcggtcagcagatcgattttatcaccgaacagaacgtgtatgattatcgtagcattaccagcattggtaaaggctatgacagcattaccgaactgcattttaatgcctttaatggtagcgcaaaaggcaccgaagttctgattcagagcagcctggaagcagataaagaagatatggcaattctgagcctgctgagccgttattttcagaatcgtggtatcaaaaaagtggattggctgtataatgcaaatcaggcagcaagccgtggttatacctatcgtctggttgaaattgccttcatcgataacgaacaggatatggccatctttgaaaacaaaaaagaggatattgccaaaggtctggttagcgcaattaccggtgttgaagttaaaaccattgttccgagccctccgagcagcaccgttggtagcagcggcaccccgagcaaaagcatttatctggttggtgatagcctgcgtgttctgccgcatgcaacccattatcagaccggtcagaaaattgcaaattgggttaaaggtcgcacctacaaaattctgcaggttaaaaatgtgcatcagagcaatagcaaacgtgcatatctgctggatggtattaaaagctgggttctggaacaggatgttgaaggcaccacc

SEQ ID NO: 8 - DNA, 444 bp, synthetic construct
  source 1..444: mol_type = other DNA
accaccgttaatgaagccctgaataatgttcgtgcacaggttggtagcggtgttagcgttggtaatggtgaatgttatgcactggcaagctggtatgaacgtatgattagtccggatgcaaccgttggtctgggtgccggtgttggttgggttagcggtgcaattggtgataccattagcgcaaaaaacattggcagcagctataattggcaggcaaatggttggaccgttagcaccagcggtccgtttaaagcaggtcagattgttaccctgggtgcaacaccgggtaatccgtatggtcatgttgttattgttgaagccgttgatggtgatcgtctgaccattctggaacagaattatggtggtaaacgttatccggtgcgtaactattattcagcagcaagctatcgtcagcaggttgttcattatatcacccctccgggt

SEQ ID NO: 9 - protein, 557 aa, synthetic construct
  UNSURE 160..161: note = XX can be "GA", "AG", "GS" or "KY"
  UNSURE 289..290: note = XX can be "GA", "AG", "GS" or "KY"
  UNSURE 556..557: note = XX can be "GA", "AG", "GS" or "KY"
TTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGXXIDQLVQETLAGKYGNGEQRKAALGSQYQAVMAVINGKATAPKKTVDQLAQEVIQGKHGNGEDRKKSLGPDYDAVQKRVTEILKGSPSGNIPKTPSDAPKSEVVNSSTEPKTEETGANGKATDTKITKXXGKHLVICGHGQGRTGYDPGAVNAKLGITEAGKVRELSKLMSKYSGQQIDFITEQNVYDYRSITSIGKGYDSITELHFNAFNGSAKGTEVLIQSSLEADKEDMAILSLLSRYFQNRGIKKVDWLYNANQAASRGYTYRLVEIAFIDNEQDMAIFENKKEDIAKGLVSAITGVEVKTIVPSPPSSTVGSSGTPSKSIYLVGDSLRVLPHATHYQTGQKIANWVKGRTYKILQVKNVHQSNSKRAYLLDGIKSWVLEQDVEGTTKGHSXX

SEQ ID NO: 10 - DNA, 1671 bp, synthetic construct
  source 1..1671: mol_type = other DNA
  unsure 478..483: note = nnnnnn can be "GGTGCT", "GCAGGC", "GGAAGC" or "AAGTAC"
  unsure 865..870: note = nnnnnn can be "GGTGCT", "GCAGGC", "GGAAGC" or "AAGTAC"
  unsure 1666..1671: note = nnnnnn can be "GGTGCT", "GCAGGC", "GGAAGC" or "AAGTAC"
accaccgttaatgaagccctgaataatgttcgtgcacaggttggtagcggtgttagcgttggtaatggtgaatgttatgcactggcaagctggtatgaacgtatgattagtccggatgcaaccgttggtctgggtgccggtgttggttgggttagcggtgcaattggtgataccattagcgcaaaaaacattggcagcagctataattggcaggcaaatggttggaccgttagcaccagcggtccgtttaaagcaggtcagattgttaccctgggtgcaacaccgggtaatccgtatggtcatgttgttattgttgaagccgttgatggtgatcgtctgaccattctggaacagaattatggtggtaaacgttatccggtgcgtaactattattcagcagcaagctatcgtcagcaggttgttcattatatcacccctccgggtacagttgcacagagcgcaccgaatctggcaggtnnnnnnatcgatcagctggtacaggaaaccttagctggcaagtatggcaatggcgagcaacgcaaagcggccctgggtagtcagtaccaagcagtgatggcggtgattaacggtaaggcgacagccccgaagaaaaccgtcgatcagttagcgcaagaggtcattcaaggcaagcacggtaatggcgaggatcgcaagaagtcgctgggccccgattacgatgcggtacagaagcgcgtcaccgagattttgaagggctcgcctagtggcaacattcctaagactccgtcggatgcccctaagtcagaagtagtgaactcaagtacggagcccaaaaccgaagaaaccggcgcgaacggcaaggccaccgacactaagattaccaagnnnnnnggcaaacatctggtgatttgtggtcatggtcagggtcgtaccggttatgatccgggtgcagttaatgcaaaactgggtattaccgaagcaggtaaagttcgtgaactgagcaaactgatgagcaaatatagcggtcagcagatcgattttatcaccgaacagaacgtgtatgattatcgtagcattaccagcattggtaaaggctatgacagcattaccgaactgcattttaatgcctttaatggtagcgcaaaaggcaccgaagttctgattcagagcagcctggaagcagataaagaagatatggcaattctgagcctgctgagccgttattttcagaatcgtggtatcaaaaaagtggattggctgtataatgcaaatcaggcagcaagccgtggttatacctatcgtctggttgaaattgccttcatcgataacgaacaggatatggccatctttgaaaacaaaaaagaggatattgccaaaggtctggttagcgcaattaccggtgttgaagttaaaaccattgttccgagccctccgagcagcaccgttggtagcagcggcaccccgagcaaaagcatttatctggttggtgatagcctgcgtgttctgccgcatgcaacccattatcagaccggtcagaaaattgcaaattgggttaaaggtcgcacctacaaaattctgcaggttaaaaatgtgcatcagagcaatagcaaacgtgcatatctgctggatggtattaaaagctgggttctggaacaggatgttgaaggcaccaccaaaggtcatagcnnnnnn

SEQ ID NO: 11 - protein, 278 aa, synthetic construct
MKETWWETWWTEWSQPKKKRKVGATTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGAGSRSYRETGTMTVTVDALNVRRAPNTSGEIVAVYKRGESFDYDTVIIDVNGYVWVSYIGGSGKRNYVATGATKDGKRFGNAWGTFKGSHHHHHH

SEQ ID NO: 12 - protein, 374 aa, synthetic construct
MYGRKKRRQRRRGAGKHLVICGHGQGRTGYDPGAVNAKLGITEAGKVRELSKLMSKYSGQQIDFITEQNVYDYRSITSIGKGYDSITELHFNAFNGSAKGTEVLIQSSLEADKEDMAILSLLSRYFQNRGIKKVDWLYNANQAASRGYTYRLVEIAFIDNEQDMAIFENKKEDIAKGLVSAITGVEVKTIVPSPPSSTVGSSGTPSKSIYLVGDSLRVLPHATHYQTGQKIANWVKGRTYKILQVKNVHQSNSKRAYLLDGIKSWVLEQDVEGTTKGHSAGSRSYRETGTMTVTVDALNVRRAPNTSGEIVAVYKRGESFDYDTVIIDVNGYVWVSYIGGSGKRNYVATGATKDGKRFGNAWGTFKGSHHHHHH

SEQ ID NO: 13 - protein, 574 aa, synthetic construct
MRRRRRRRRGATTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGAGIDQLVQETLAGKYGNGEQRKAALGSQYQAVMAVINGKATAPKKTVDQLAQEVIQGKHGNGEDRKKSLGPDYDAVQKRVTEILKGSPSGNIPKTPSDAPKSEVVNSSTEPKTEETGANGKATDTKITKGSGKHLVICGHGQGRTGYDPGAVNAKLGITEAGKVRELSKLMSKYSGQQIDFITEQNVYDYRSITSIGKGYDSITELHFNAFNGSAKGTEVLIQSSLEADKEDMAILSLLSRYFQNRGIKKVDWLYNANQAASRGYTYRLVEIAFIDNEQDMAIFENKKEDIAKGLVSAITGVEVKTIVPSPPSSTVGSSGTPSKSIYLVGDSLRVLPHATHYQTGQKIANWVKGRTYKILQVKNVHQSNSKRAYLLDGIKSWVLEQDVEGTTKGHSKYHHHHHH

SEQ ID NO: 14 - protein, 532 aa, synthetic construct
MRRRRRRRRGATTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGAGSRSYRETGTMTVTVDALNVRRAPNTSGEIVAVYKRGESFDYDTVIIDVNGYVWVSYIGGSGKRNYVATGATKDGKRFGNAWGTFKGSGKHLVICGHGQGRTGYDPGAVNAKLGITEAGKVRELSKLMSKYSGQQIDFITEQNVYDYRSITSIGKGYDSITELHFNAFNGSAKGTEVLIQSSLEADKEDMAILSLLSRYFQNRGIKKVDWLYNANQAASRGYTYRLVEIAFIDNEQDMAIFENKKEDIAKGLVSAITGVEVKTIVPSPPSSTVGSSGTPSKSIYLVGDSLRVLPHATHYQTGQKIANWVKGRTYKILQVKNVHQSNSKRAYLLDGIKSWVLEQDVEGTTKGHSKYHHHHHH

SEQ ID NO: 15 - protein, 606 aa, synthetic construct
MGFGCNGPWNEDDLRCRNHCKSIKGYKGGYCAKGGFVCKCYGATTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGAGIDQLVQETLAGKYGNGEQRKAALGSQYQAVMAVINGKATAPKKTVDQLAQEVIQGKHGNGEDRKKSLGPDYDAVQKRVTEILKGSPSGNIPKTPSDAPKSEVVNSSTEPKTEETGANGKATDTKITKGSGKHLVICGHGQGRTGYDPGAVNAKLGITEAGKVRELSKLMSKYSGQQIDFITEQNVYDYRSITSIGKGYDSITELHFNAFNGSAKGTEVLIQSSLEADKEDMAILSLLSRYFQNRGIKKVDWLYNANQAASRGYTYRLVEIAFIDNEQDMAIFENKKEDIAKGLVSAITGVEVKTIVPSPPSSTVGSSGTPSKSIYLVGDSLRVLPHATHYQTGQKIANWVKGRTYKILQVKNVHQSNSKRAYLLDGIKSWVLEQDVEGTTKGHSKYHHHHHH

SEQ ID NO: 16 - protein, 535 aa, synthetic construct
MYGRKKRRQRRRGATTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGAGSRSYRETGTMTVTVDALNVRRAPNTSGEIVAVYKRGESFDYDTVIIDVNGYVWVSYIGGSGKRNYVATGATKDGKRFGNAWGTFKGSGKHLVICGHGQGRTGYDPGAVNAKLGITEAGKVRELSKLMSKYSGQQIDFITEQNVYDYRSITSIGKGYDSITELHFNAFNGSAKGTEVLIQSSLEADKEDMAILSLLSRYFQNRGIKKVDWLYNANQAASRGYTYRLVEIAFIDNEQDMAIFENKKEDIAKGLVSAITGVEVKTIVPSPPSSTVGSSGTPSKSIYLVGDSLRVLPHATHYQTGQKIANWVKGRTYKILQVKNVHQSNSKRAYLLDGIKSWVLEQDVEGTTKGHSKYHHHHHH

SEQ ID NO: 17 - protein, 577 aa, synthetic construct
MYGRKKRRQRRRGATTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGAGIDQLVQETLAGKYGNGEQRKAALGSQYQAVMAVINGKATAPKKTVDQLAQEVIQGKHGNGEDRKKSLGPDYDAVQKRVTEILKGSPSGNIPKTPSDAPKSEVVNSSTEPKTEETGANGKATDTKITKGSGKHLVICGHGQGRTGYDPGAVNAKLGITEAGKVRELSKLMSKYSGQQIDFITEQNVYDYRSITSIGKGYDSITELHFNAFNGSAKGTEVLIQSSLEADKEDMAILSLLSRYFQNRGIKKVDWLYNANQAASRGYTYRLVEIAFIDNEQDMAIFENKKEDIAKGLVSAITGVEVKTIVPSPPSSTVGSSGTPSKSIYLVGDSLRVLPHATHYQTGQKIANWVKGRTYKILQVKNVHQSNSKRAYLLDGIKSWVLEQDVEGTTKGHSKYHHHHHH

SEQ ID NO: 18 - protein, 587 aa, synthetic construct
MKETWWETWWTEWSQPKKKRKVGATTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGAGIDQLVQETLAGKYGNGEQRKAALGSQYQAVMAVINGKATAPKKTVDQLAQEVIQGKHGNGEDRKKSLGPDYDAVQKRVTEILKGSPSGNIPKTPSDAPKSEVVNSSTEPKTEETGANGKATDTKITKGSGKHLVICGHGQGRTGYDPGAVNAKLGITEAGKVRELSKLMSKYSGQQIDFITEQNVYDYRSITSIGKGYDSITELHFNAFNGSAKGTEVLIQSSLEADKEDMAILSLLSRYFQNRGIKKVDWLYNANQAASRGYTYRLVEIAFIDNEQDMAIFENKKEDIAKGLVSAITGVEVKTIVPSPPSSTVGSSGTPSKSIYLVGDSLRVLPHATHYQTGQKIANWVKGRTYKILQVKNVHQSNSKRAYLLDGIKSWVLEQDVEGTTKGHSKYHHHHHH

SEQ ID NO: 19 - protein, 564 aa, synthetic construct
MGFGCNGPWNEDDLRCRNHCKSIKGYKGGYCAKGGFVCKCYGATTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGAGSRSYRETGTMTVTVDALNVRRAPNTSGEIVAVYKRGESFDYDTVIIDVNGYVWVSYIGGSGKRNYVATGATKDGKRFGNAWGTFKGSGKHLVICGHGQGRTGYDPGAVNAKLGITEAGKVRELSKLMSKYSGQQIDFITEQNVYDYRSITSIGKGYDSITELHFNAFNGSAKGTEVLIQSSLEADKEDMAILSLLSRYFQNRGIKKVDWLYNANQAASRGYTYRLVEIAFIDNEQDMAIFENKKEDIAKGLVSAITGVEVKTIVPSPPSSTVGSSGTPSKSIYLVGDSLRVLPHATHYQTGQKIANWVKGRTYKILQVKNVHQSNSKRAYLLDGIKSWVLEQDVEGTTKGHSKYHHHHHH

SEQ ID NO: 20 - protein, 581 aa, synthetic construct
MRQIKWFQNRRMKWKKGATTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGAGIDQLVQETLAGKYGNGEQRKAALGSQYQAVMAVINGKATAPKKTVDQLAQEVIQGKHGNGEDRKKSLGPDYDAVQKRVTEILKGSPSGNIPKTPSDAPKSEVVNSSTEPKTEETGANGKATDTKITKGSGKHLVICGHGQGRTGYDPGAVNAKLGITEAGKVRELSKLMSKYSGQQIDFITEQNVYDYRSITSIGKGYDSITELHFNAFNGSAKGTEVLIQSSLEADKEDMAILSLLSRYFQNRGIKKVDWLYNANQAASRGYTYRLVEIAFIDNEQDMAIFENKKEDIAKGLVSAITGVEVKTIVPSPPSSTVGSSGTPSKSIYLVGDSLRVLPHATHYQTGQKIANWVKGRTYKILQVKNVHQSNSKRAYLLDGIKSWVLEQDVEGTTKGHSKYHHHHHH

SEQ ID NO: 21 - protein, 497 aa, synthetic construct
MKETWWETWWTEWSQPKKKRKVGATTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGAGSRSYRETGTMTVTVDALNVRRAPNTSGEIVAVYKRGESFDYDTVIIDVNGYVWVSYIGGSGKRNYVATGATKDGKRFGNAWGTFKGSTLKVSKNHINYTMDKRGKKPEGMVIHNDAGRSSGQQYENSLANAGYARYANGIAHYYGSEGYVWEAIDAKNQIAWHTGDGTGANSGNFRFAGIEVCQSMSASDAQFLKNEQAVFQFTAEKFKEWGLTPNRKTVRLHMEFVPTACPHRSMVLHTGFNPVTQGRPSQAIMNKLKDYFIKQIKNYMDKGTSSSTIVKDGKTSSASTPATRPVTGSWKKNQKYHHHHHH

SEQ ID NO: 22 - protein, 532 aa, synthetic construct
MRRRRRRRRGATTVNEALNNVRAQVGSGVSVGNGECYALASWYERMISPDATVGLGAGVGWVSGAIGDTISAKNIGSSYNWQANGWTVSTSGPFKAGQIVTLGATPGNPYGHVVIVEAVDGDRLTILEQNYGGKRYPVRNYYSAASYRQQVVHYITPPGTVAQSAPNLAGAGGKHLVICGHGQGRTGYDPGAVNAKLGITEAGKVRELSKLMSKYSGQQIDFITEQNVYDYRSITSIGKGYDSITELHFNAFNGSAKGTEVLIQSSLEADKEDMAILSLLSRYFQNRGIKKVDWLYNANQAASRGYTYRLVEIAFIDNEQDMAIFENKKEDIAKGLVSAITGVEVKTIVPSPPSSTVGSSGTPSKSIYLVGDSLRVLPHATHYQTGQKIANWVKGRTYKILQVKNVHQSNSKRAYLLDGIKSWVLEQDVEGTTKGHSGSSRSYRETGTMTVTVDALNVRRAPNTSGEIVAVYKRGESFDYDTVIIDVNGYVWVSYIGGSGKRNYVATGATKDGKRFGNAWGTFKKYHHHHHH

SEQ ID NO: 23 - DNA, 837 bp, synthetic construct
  source 1..837: mol_type = other DNA
atgaaggagacttggtgggaaacctggtggacggaatggagccaacccaagaagaagcgtaaagtcggtgctaccaccgttaatgaagccctgaataatgttcgtgcacaggttggtagcggtgttagcgttggtaatggtgaatgttatgcactggcaagctggtatgaacgtatgattagtccggatgcaaccgttggtctgggtgccggtgttggttgggttagcggtgcaattggtgataccattagcgcaaaaaacattggcagcagctataattggcaggcaaatggttggaccgttagcaccagcggtccgtttaaagcaggtcagattgttaccctgggtgcaacaccgggtaatccgtatggtcatgttgttattgttgaagccgttgatggtgatcgtctgaccattctggaacagaattatggtggtaaacgttatccggtgcgtaactattattcagcagcaagctatcgtcagcaggttgttcattatatcacccctccgggtacagttgcacagagcgcaccgaatctggcaggtgcaggcagccgtagctatcgtgaaaccggcaccatgaccgttaccgttgatgcactgaatgttcgtcgtgcaccgaataccagcggtgaaattgttgcagtgtataaacgtggtgagagcttcgattatgataccgtgattattgatgtgaacggttatgtttgggtgagctatattggtggttcaggcaaacgtaattatgttgcaaccggtgccaccaaagatggtaaacgctttggtaatgcatggggcacctttaaaggaagccaccatcatcatcatcattaa

SEQ ID NO: 24 - DNA, 1125 bp, synthetic construct
  source 1..1125: mol_type = other DNA
atgtatggacgcaagaagcgtcgccaacgtcgccgtggtgctggcaaacatctggtgatttgtggtcatggtcagggtcgtaccggttatgatccgggtgcagttaatgcaaaactgggtattaccgaagcaggtaaagttcgtgaactgagcaaactgatgagcaaatatagcggtcagcagatcgattttatcaccgaacagaacgtgtatgattatcgtagcattaccagcattggtaaaggctatgacagcattaccgaactgcattttaatgcctttaatggtagcgcaaaaggcaccgaagttctgattcagagcagcctggaagcagataaagaagatatggcaattctgagcctgctgagccgttattttcagaatcgtggtatcaaaaaagtggattggctgtataatgcaaatcaggcagcaagccgtggttatacctatcgtctggttgaaattgccttcatcgataacgaacaggatatggccatctttgaaaacaaaaaagaggatattgccaaaggtctggttagcgcaattaccggtgttgaagttaaaaccattgttccgagccctccgagcagcaccgttggtagcagcggcaccccgagcaaaagcatttatctggttggtgatagcctgcgtgttctgccgcatgcaacccattatcagaccggtcagaaaattgcaaattgggttaaaggtcgcacctacaaaattctgcaggttaaaaatgtgcatcagagcaatagcaaacgtgcatatctgctggatggtattaaaagctgggttctggaacaggatgttgaaggcaccaccaaaggtcatagcgcaggcagccgtagctatcgtgaaaccggcaccatgaccgttaccgttgatgcactgaatgttcgtcgtgcaccgaataccagcggtgaaattgttgcagtgtataaacgtggtgagagcttcgattatgataccgtgattattgatgtgaacggttatgtttgggtgagctatattggtggttcaggcaaacgtaattatgttgcaaccggtgccaccaaagatggtaaacgctttggtaatgcatggggcacctttaaaggaagccaccatcatcatcatcattaa

SEQ ID NO: 25 - DNA, 1725 bp, synthetic construct
  source 1..1725: mol_type = other DNA
atgcgccgtcgtcgccgccgtcgtcgcggtgctaccaccgttaatgaagccctgaataatgttcgtgcacaggttggtagcggtgttagcgttggtaatggtgaatgttatgcactggcaagctggtatgaacgtatgattagtccggatgcaaccgttggtctgggtgccggtgttggttgggttagcggtgcaattggtgataccattagcgcaaaaaacattggcagcagctataattggcaggcaaatggttggaccgttagcaccagcggtccgtttaaagcaggtcagattgttaccctgggtgcaacaccgggtaatccgtatggtcatgttgttattgttgaagccgttgatggtgatcgtctgaccattctggaacagaattatggtggtaaacgttatccggtgcgtaactattattcagcagcaagctatcgtcagcaggttgttcattatatcacccctccgggtacagttgcacagagcgcaccgaatctggcaggtgcaggcatcgatcagctggtacaggaaaccttagctggcaagtatggcaatggcgagcaacgcaaagcggccctgggtagtcagtaccaagcagtgatggcggtgattaacggtaaggcgacagccccgaagaaaaccgtcgatcagttagcgcaagaggtcattcaaggcaagcacggtaatggcgaggatcgcaagaagtcgctgggccccgattacgatgcggtacagaagcgcgtcaccgagattttgaagggctcgcctagtggcaacattcctaagactccgtcggatgcccctaagtcagaagtagtgaactcaagtacggagcccaaaaccgaagaaaccggcgcgaacggcaaggccaccgacactaagattaccaagggaagcggcaaacatctggtgatttgtggtcatggtcagggtcgtaccggttatgatccgggtgcagttaatgcaaaactgggtattaccgaagcaggtaaagttcgtgaactgagcaaactgatgagcaaatatagcggtcagcagatcgattttatcaccgaacagaacgtgtatgattatcgtagcattaccagcattggtaaaggctatgacagcattaccgaactgcattttaatgcctttaatggtagcgcaaaaggcaccgaagttctgattcagagcagcctggaagcagataaagaagatatggcaattctgagcctgctgagccgttattttcagaatcgtggtatcaaaaaagtggattggctgtataatgcaaatcaggcagcaagccgtggttatacctatcgtctggttgaaattgccttcatcgataacgaacaggatatggccatctttgaaaacaaaaaagaggatattgccaaaggtctggttagcgcaattaccggtgttgaagttaaaaccattgttccgagccctccgagcagcaccgttggtagcagcggcaccccgagcaaaagcatttatctggttggtgatagcctgcgtgttctgccgcatgcaacccattatcagaccggtcagaaaattgcaaattgggttaaaggtcgcacctacaaaattctgcaggttaaaaatgtgcatcagagcaatagcaaacgtgcatatctgctggatggtattaaaagctgggttctggaacaggatgttgaaggcaccaccaaaggtcatagcaagtaccaccatcatcatcatcattaa

SEQ ID NO: 26 - DNA, 1599 bp, synthetic construct
  source 1..1599: mol_type = other DNA
atgcgccgtcgtcgccgccgtcgtcgcggtgctaccaccgttaatgaagccctgaataatgttcgtgcacaggttggtagcggtgttagcgttggtaatggtgaatgttatgcactggcaagctggtatgaacgtatgattagtccggatgcaaccgttggtctgggtgccggtgttggttgggttagcggtgcaattggtgataccattagcgcaaaaaacattggcagcagctataattggcaggcaaatggttggaccgttagcaccagcggtccgtttaaagcaggtcagattgttaccctgggtgcaacaccgggtaatccgtatggtcatgttgttattgttgaagccgttgatggtgatcgtctgaccattctggaacagaattatggtggtaaacgttatccggtgcgtaactattattcagcagcaagctatcgtcagcaggttgttcattatatcacccctccgggtacagttgcacagagcgcaccgaatctggcaggtgcaggcagccgtagctatcgtgaaaccggcaccatgaccgttaccgttgatgcactgaatgttcgtcgtgcaccgaataccagcggtgaaattgttgcagtgtataaacgtggtgagagcttcgattatgataccgtgattattgatgtgaacggttatgtttgggtgagctatattggtggttcaggcaaacgtaattatgttgcaaccggtgccaccaaagatggtaaacgctttggtaatgcatggggcacctttaaaggaagcggcaaacatctggtgatttgtggtcatggtcagggtcgtaccggttatgatccgggtgcagttaatgcaaaactgggtattaccgaagcaggtaaagttcgtgaactgagcaaactgatgagcaaatatagcggtcagcagatcgattttatcaccgaacagaacgtgtatgattatcgtagcattaccagcattggtaaaggctatgacagcattaccgaactgcattttaatgcctttaatggtagcgcaaaaggcaccgaagttctgattcagagcagcctggaagcagataaagaagatatggcaattctgagcctgctgagccgttattttcagaatcgtggtatcaaaaaagtggattggctgtataatgcaaatcaggcagcaagccgtggttatacctatcgtctggttgaaattgccttcatcgataacgaacaggatatggccatctttgaaaacaaaaaagaggatattgccaaaggtctggttagcgcaattaccggtgttgaagttaaaaccattgttccgagccctccgagcagcaccgttggtagcagcggcaccccgagcaaaagcatttatctggttggtgatagcctgcgtgttctgccgcatgcaacccattatcagaccggtcagaaaattgcaaattgggttaaaggtcgcacctacaaaattctgcaggttaaaaatgtgcatcagagcaatagcaaacgtgcatatctgctggatggtattaaaagctgggttctggaacaggatgttgaaggcaccaccaaaggtcatagcaagtaccaccatcatcatcatcattaa

SEQ ID NO: 27 - DNA, 1821 bp, synthetic construct
  source 1..1821: mol_type = other DNA
atgggttttggttgtaatggtccctggaatgaggacgatttgcgctgccgtaatcattgtaagtcaattaagggatataagggcggttattgtgcaaagggtggttttgtttgtaaatgttacggtgctaccaccgttaatgaagccctgaataatgttcgtgcacaggttggtagcggtgttagcgttggtaatggtgaatgttatgcactggcaagctggtatgaacgtatgattagtccggatgcaaccgttggtctgggtgccggtgttggttgggttagcggtgcaattggtgataccattagcgcaaaaaacattggcagcagctataattggcaggcaaatggttggaccgttagcaccagcggtccgtttaaagcaggtcagattgttaccctgggtgcaacaccgggtaatccgtatggtcatgttgttattgttgaagccgttgatggtgatcgtctgaccattctggaacagaattatggtggtaaacgttatccggtgcgtaactattattcagcagcaagctatcgtcagcaggttgttcattatatcacccctccgggtacagttgcacagagcgcaccgaatctggcaggtgcaggcatcgatcagctggtacaggaaaccttagctggcaagtatggcaatggcgagcaacgcaaagcggccctgggtagtcagtaccaagcagtgatggcggtgattaacggtaaggcgacagccccgaagaaaaccgtcgatcagttagcgcaagaggtcattcaaggcaagcacggtaatggcgaggatcgcaagaagtcgctgggccccgattacgatgcggtacagaagcgcgtcaccgagattttgaagggctcgcctagtggcaacattcctaagactccgtcggatgcccctaagtcagaagtagtgaactcaagtacggagcccaaaaccgaagaaaccggcgcgaacggcaaggccaccgacactaagattaccaagggaagcggcaaacatctggtgatttgtggtcatggtcagggtcgtaccggttatgatccgggtgcagttaatgcaaaactgggtattaccgaagcaggtaaagttcgtgaactgagcaaactgatgagcaaatatagcggtcagcagatcgattttatcaccgaacagaacgtgtatgattatcgtagcattaccagcattggtaaaggctatgacagcattaccgaactgcattttaatgcctttaatggtagcgcaaaaggcaccgaagttctgattcagagcagcctggaagcagataaagaagatatggcaattctgagcctgctgagccgttattttcagaatcgtggtatcaaaaaagtggattggctgtataatgcaaatcaggcagcaagccgtggttatacctatcgtctggttgaaattgccttcatcgataacgaacaggatatggccatctttgaaaacaaaaaagaggatattgccaaaggtctggttagcgcaattaccggtgttgaagttaaaaccattgttccgagccctccgagcagcaccgttggtagcagcggcaccccgagcaaaagcatttatctggttggtgatagcctgcgtgttctgccgcatgcaacccattatcagaccggtcagaaaattgcaaattgggttaaaggtcgcacctacaaaattctgcaggttaaaaatgtgcatcagagcaatagcaaacgtgcatatctgctggatggtattaaaagctgggttctggaacaggatgttgaaggcaccaccaaaggtcatagcaagtaccaccatcatcatcatcattaa

SEQ ID NO: 28 - DNA, 1608 bp, synthetic construct
  source 1..1608: mol_type = other DNA
atgtatggacgcaagaagcgtcgccaacgtcgccgtggtgctaccaccgttaatgaagccctgaataatgttcgtgcacaggttggtagcggtgttagcgttggtaatggtgaatgttatgcactggcaagctggtatgaacgtatgattagtccggatgcaaccgttggtctgggtgccggtgttggttgggttagcggtgcaattggtgataccattagcgcaaaaaacattggcagcagctataattggcaggcaaatggttggaccgttagcaccagcggtccgtttaaagcaggtcagattgttaccctgggtgcaacaccgggtaatccgtatggtcatgttgttattgttgaagccgttgatggtgatcgtctgaccattctggaacagaattatggtggtaaacgttatccggtgcgtaactattattcagcagcaagctatcgtcagcaggttgttcattatatcacccctccgggtacagttgcacagagcgcaccgaatctggcaggtgcaggcagccgtagctatcgtgaaaccggcaccatgaccgttaccgttgatgcactgaatgttcgtcgtgcaccgaataccagcggtgaaattgttgcagtgtataaacgtggtgagagcttcgattatgataccgtgattattgatgtgaacggttatgtttgggtgagctatattggtggttcaggcaaacgtaattatgttgcaaccggtgccaccaaagatggtaaacgctttggtaatgcatggggcacctttaaaggaagcggcaaacatctggtgatttgtggtcatggtcagggtcgtaccggttatgatccgggtgcagttaatgcaaaactgggtattaccgaagcaggtaaagttcgtgaactgagcaaactgatgagcaaatatagcggtcagcagatcgattttatcaccgaacagaacgtgtatgattatcgtagcattaccagcattggtaaaggctatgacagcattaccgaactgcattttaatgcctttaatggtagcgcaaaaggcaccgaagttctgattcagagcagcctggaagcagataaagaagatatggcaattctgagcctgctgagccgttattttcagaatcgtggtatcaaaaaagtggattggctgtataatgcaaatcaggcagcaagccgtggttatacctatcgtctggttgaaattgccttcatcgataacgaacaggatatggccatctttgaaaacaaaaaagaggatattgccaaaggtctggttagcgcaattaccggtgttgaagttaaaaccattgttccgagccctccgagcagcaccgttggtagcagcggcaccccgagcaaaagcatttatctggttggtgatagcctgcgtgttctgccgcatgcaacccattatcagaccggtcagaaaattgcaaattgggttaaaggtcgcacctacaaaattctgcaggttaaaaatgtgcatcagagcaatagcaaacgtgcatatctgctggatggtattaaaagctgggttctggaacaggatgttgaaggcaccaccaaaggtcatagcaagtaccaccatcatcatcatcattaa

SEQ ID NO: 29 - DNA, 1734 bp, synthetic construct
  source 1..1734: mol_type = other DNA
atgtatggacgcaagaagcgtcgccaacgtcgccgtggtgctaccaccgttaatgaagccctgaataatgttcgtgcacaggttggtagcggtgttagcgttggtaatggtgaatgttatgcactggcaagctggtatgaacgtatgattagtccggatgcaaccgttggtctgggtgccggtgttggttgggttagcggtgcaattggtgataccattagcgcaaaaaacattggcagcagctataattggcaggcaaatggttggaccgttagcaccagcggtccgtttaaagcaggtcagattgttaccctgggtgcaacaccgggtaatccgtatggtcatgttgttattgttgaagccgttgatggtgatcgtctgaccattctggaacagaattatggtggtaaacgttatccggtgcgtaactattattcagcagcaagctatcgtcagcaggttgttcattatatcacccctccgggtacagttgcacagagcgcaccgaatctggcaggtgcaggcatcgatcagctggtacaggaaaccttagctggcaagtatggcaatggcgagcaacgcaaagcggccctgggtagtcagtaccaagcagtgatggcggtgattaacggtaaggcgacagccccgaagaaaaccgtcgatcagttagcgcaagaggtcattcaaggcaagcacggtaatggcgaggatcgcaagaagtcgctgggccccgattacgatgcggtacagaagcgcgtcaccgagattttgaagggctcgcctagtggcaacattcctaagactccgtcggatgcccctaagtcagaagtagtgaactcaagtacggagcccaaaaccgaagaaaccggcgcgaacggcaaggccaccgacactaagattaccaagggaagcggcaaacatctggtgatttgtggtcatggtcagggtcgtaccggttatgatccgggtgcagttaatgcaaaactgggtattaccgaagcaggtaaagttcgtgaactgagcaaactgatgagcaaatatagcggtcagcagatcgattttatcaccgaacagaacgtgtatgattatcgtagcattaccagcattggtaaaggctatgacagcattaccgaactgcattttaatgcctttaatggtagcgcaaaaggcaccgaagttctgattcagagcagcctggaagcagataaagaagatatggcaattctgagcctgctgagccgttattttcagaatcgtggtatcaaaaaagtggattggctgtataatgcaaatcaggcagcaagccgtggttatacctatcgtctggttgaaattgccttcatcgataacgaacaggatatggccatctttgaaaacaaaaaagaggatattgccaaaggtctggttagcgcaattaccggtgttgaagttaaaaccattgttccgagccctccgagcagcaccgttggtagcagcggcaccccgagcaaaagcatttatctggttggtgatagcctgcgtgttctgccgcatgcaacccattatcagaccggtcagaaaattgcaaattgggttaaaggtcgcacctacaaaattctgcaggttaaaaatgtgcatcagagcaatagcaaacgtgcatatctgctggatggtattaaaagctgggttctggaacaggatgttgaaggcaccaccaaaggtcatagcaagtaccaccatcatcatcatcattaa

SEQ ID NO: 30 - DNA, 1764 bp, synthetic construct
  source 1..1764: mol_type = other DNA
atgaaggagacttggtgggaaacctggtggacggaatggagccaacccaagaagaagcgtaaagtcggtgctaccaccgttaatgaagccctgaataatgttcgtgcacaggttggtagcggtgttagcgttggtaatggtgaatgttatgcactggcaagctggtatgaacgtatgattagtccggatgcaaccgttggtctgggtgccggtgttggttgggttagcggtgcaattggtgataccattagcgcaaaaaacattggcagcagctataattggcaggcaaatggttggaccgttagcaccagcggtccgtttaaagcaggtcagattgttaccctgggtgcaacaccgggtaatccgtatggtcatgttgttattgttgaagccgttgatggtgatcgtctgaccattctggaacagaattatggtggtaaacgttatccggtgcgtaactattattcagcagcaagctatcgtcagcaggttgttcattatatcacccctccgggtacagttgcacagagcgcaccgaatctggcaggtgcaggcatcgatcagctggtacaggaaaccttagctggcaagtatggcaatggcgagcaacgcaaagcggccctgggtagtcagtaccaagcagtgatggcggtgattaacggtaaggcgacagccccgaagaaaaccgtcgatcagttagcgcaagaggtcattcaaggcaagcacggtaatggcgaggatcgcaagaagtcgctgggccccgattacgatgcggtacagaagcgcgtcaccgagattttgaagggctcgcctagtggcaacattcctaagactccgtcggatgcccctaagtcagaagtagtgaactcaagtacggagcccaaaaccgaagaaaccggcgcgaacggcaaggccaccgacactaagattaccaagggaagcggcaaacatctggtgatttgtggtcatggtcagggtcgtaccggttatgatccgggtgcagttaatgcaaaactgggtattaccgaagcaggtaaagttcgtgaactgagcaaactgatgagcaaatatagcggtcagcagatcgattttatcaccgaacagaacgtgtatgattatcgtagcattaccagcattggtaaaggctatgacagcattaccgaactgcattttaatgcctttaatggtagcgcaaaaggcaccgaagttctgattcagagcagcctggaagcagataaagaagatatggcaattctgagcctgctgagccgttattttcagaatcgtggtatcaaaaaagtggattggctgtataatgcaaatcaggcagcaagccgtggttatacctatcgtctggttgaaattgccttcatcgataacgaacaggatatggccatctttgaaaacaaaaaagaggatattgccaaaggtctggttagcgcaattaccggtgttgaagttaaaaccattgttccgagccctccgagcagcaccgttggtagcagcggcaccccgagcaaaagcatttatctggttggtgatagcctgcgtgttctgccgcatgcaacccattatcagaccggtcagaaaattgcaaattgggttaaaggtcgcacctacaaaattctgcaggttaaaaatgtgcatcagagcaatagcaaacgtgcatatctgctggatggtattaaaagctgggttctggaacaggatgttgaaggcaccaccaaaggtcatagcaagtaccaccatcatcatcatcattaa

SEQ ID NO: 31 - DNA, 1695 bp, synthetic construct
  source 1..1695: mol_type = other DNA
atgggttttggttgtaatggtccctggaatgaggacgatttgcgctgccgtaatcattgtaagtcaattaagggatataagggcggttattgtgcaaagggtggttttgtttgtaaatgttacggtgctaccaccgttaatgaagccctgaataatgttcgtgcacaggttggtagcggtgttagcgttggtaatggtgaatgttatgcactggcaagctggtatgaacgtatgattagtccggatgcaaccgttggtctgggtgccggtgttggttgggttagcggtgcaattggtgataccattagcgcaaaaaacattggcagcagctataattggcaggcaaatggttggaccgttagcaccagcggtccgtttaaagcaggtcagattgttaccctgggtgcaacaccgggtaatccgtatggtcatgttgttattgttgaagccgttgatggtgatcgtctgaccattctggaacagaattatggtggtaaacgttatccggtgcgtaactattattcagcagcaagctatcgtcagcaggttgttcattatatcacccctccgggtacagttgcacagagcgcaccgaatctggcaggtgcaggcagccgtagctatcgtgaaaccggcaccatgaccgttaccgttgatgcactgaatgttcgtcgtgcaccgaataccagcggtgaaattgttgcagtgtataaacgtggtgagagcttcgattatgataccgtgattattgatgtgaacggttatgtttgggtgagctatattggtggttcaggcaaacgtaattatgttgcaaccggtgccaccaaagatggtaaacgctttggtaatgcatggggcacctttaaaggaagcggcaaacatctggtgatttgtggtcatggtcagggtcgtaccggttatgatccgggtgcagttaatgcaaaactgggtattaccgaagcaggtaaagttcgtgaactgagcaaactgatgagcaaatatagcggtcagcagatcgattttatcaccgaacagaacgtgtatgattatcgtagcattaccagcattggtaaaggctatgacagcattaccgaactgcattttaatgcctttaatggtagcgcaaaaggcaccgaagttctgattcagagcagcctggaagcagataaagaagatatggcaattctgagcctgctgagccgttattttcagaatcgtggtatcaaaaaagtggattggctgtataatgcaaatcaggcagcaagccgtggttatacctatcgtctggttgaaattgccttcatcgataacgaacaggatatggccatctttgaaaacaaaaaagaggatattgccaaaggtctggttagcgcaattaccggtgttgaagttaaaaccattgttccgagccctccgagcagcaccgttggtagcagcggcaccccgagcaaaagcatttatctggttggtgatagcctgcgtgttctgccgcatgcaacccattatcagaccggtcagaaaattgcaaattgggttaaaggtcgcacctacaaaattctgcaggttaaaaatgtgcatcagagcaatagcaaacgtgcatatctgctggatggtattaaaagctgggttctggaacaggatgttgaaggcaccaccaaaggtcatagcaagtaccaccatcatcatcatcattaa

SEQ ID NO: 32 - DNA, 1746 bp, synthetic construct
  source 1..1746: mol_type = other DNA
atgcgccaaatcaagtggttccagaaccgccgtatgaaatggaagaaaggtgctaccaccgttaatgaagccctgaataatgttcgtgcacaggttggtagcggtgttagcgttggtaatggtgaatgttatgcactggcaagctggtatgaacgtatgattagtccggatgcaaccgttggtctgggtgccggtgttggttgggttagcggtgcaattggtgataccattagcgcaaaaaacattggcagcagctataattggcaggcaaatggttggaccgttagcaccagcggtccgtttaaagcaggtcagattgttaccctgggtgcaacaccgggtaatccgtatggtcatgttgttattgttgaagccgttgatggtgatcgtctgaccattctggaacagaattatggtggtaaacgttatccggtgcgtaactattattcagcagcaagctatcgtcagcaggttgttcattatatcacccctccgggtacagttgcacagagcgcaccgaatctggcaggtgcaggcatcgatcagctggtacaggaaaccttagctggcaagtatggcaatggcgagcaacgcaaagcggccctgggtagtcagtaccaagcagtgatggcggtgattaacggtaaggcgacagccccgaagaaaaccgtcgatcagttagcgcaagaggtcattcaaggcaagcacggtaatggcgaggatcgcaagaagtcgctgggccccgattacgatgcggtacagaagcgcgtcaccgagattttgaagggctcgcctagtggcaacattcctaagactccgtcggatgcccctaagtcagaagtagtgaactcaagtacggagcccaaaaccgaagaaaccggcgcgaacggcaaggccaccgacactaagattaccaagggaagcggcaaacatctggtgatttgtggtcatggtcagggtcgtaccggttatgatccgggtgcagttaatgcaaaactgggtattaccgaagcaggtaaagttcgtgaactgagcaaactgatgagcaaatatagcggtcagcagatcgattttatcaccgaacagaacgtgtatgattatcgtagcattaccagcattggtaaaggctatgacagcattaccgaactgcattttaatgcctttaatggtagcgcaaaaggcaccgaagttctgattcagagcagcctggaagcagataaagaagatatggcaattctgagcctgctgagccgttattttcagaatcgtggtatcaaaaaagtggattggctgtataatgcaaatcaggcagcaagccgtggttatacctatcgtctggttgaaattgccttcatcgataacgaacaggatatggccatctttgaaaacaaaaaagaggatattgccaaaggtctggttagcgcaattaccggtgttgaagttaaaaccattgttccgagccctccgagcagcaccgttggtagcagcggcaccccgagcaaaagcatttatctggttggtgatagcctgcgtgttctgccgcatgcaacccattatcagaccggtcagaaaattgcaaattgggttaaaggtcgcacctacaaaattctgcaggttaaaaatgtgcatcagagcaatagcaaacgtgcatatctgctggatggtattaaaagctgggttctggaacaggatgttgaaggcaccaccaaaggtcatagcaagtaccaccatcatcatcatcattaa

SEQ ID NO: 33 - DNA, 1494 bp, synthetic construct
  source 1..1494: mol_type = other DNA
atgaaggagacttggtgggaaacctggtggacggaatggagccaacccaagaagaagcgtaaagtcggtgctaccaccgttaatgaagccctgaataatgttcgtgcacaggttggtagcggtgttagcgttggtaatggtgaatgttatgcactggcaagctggtatgaacgtatgattagtccggatgcaaccgttggtctgggtgccggtgttggttgggttagcggtgcaattggtgataccattagcgcaaaaaacattggcagcagctataattggcaggcaaatggttggaccgttagcaccagcggtccgtttaaagcaggtcagattgttaccctgggtgcaacaccgggtaatccgtatggtcatgttgttattgttgaagccgttgatggtgatcgtctgaccattctggaacagaattatggtggtaaacgttatccggtgcgtaactattattcagcagcaagctatcgtcagcaggttgttcattatatcacccctccgggtacagttgcacagagcgcaccgaatctggcaggtgcaggcagccgtagctatcgtgaaaccggcaccatgaccgttaccgttgatgcactgaatgttcgtcgtgcaccgaataccagcggtgaaattgttgcagtgtataaacgtggtgagagcttcgattatgataccgtgattattgatgtgaacggttatgtttgggtgagctatattggtggttcaggcaaacgtaattatgttgcaaccggtgccaccaaagatggtaaacgctttggtaatgcatggggcacctttaaaggaagcaccctgaaagtgagcaagaaccacatcaactataccatggataagcgtggtaaaaagccggagggcatggttattcacaacgacgcgggccgtagcagcggtcagcaatacgaaaacagcctggcgaacgcgggttacgcgcgttatgcgaacggcatcgcgcactactatggcagcgagggttatgtgtgggaagcgatcgacgcgaaaaaccagattgcgtggcacaccggtgatggtaccggtgcgaacagcggtaacttccgttttgcgggcatcgaggtgtgccagagcatgagcgcgagcgatgcgcaattcctgaagaacgaacaggcggttttccaatttaccgcggagaaatttaaggaatggggtctgaccccgaaccgtaaaaccgtgcgtctgcacatggagtttgttccgaccgcgtgcccgcaccgtagcatggtgctgcacaccggttttaacccggttacccagggccgtccgagccaagcgatcatgaacaagctgaaggactacttcatcaagcaaatcaagaactacatggataagggtaccagcagcagcaccattgtgaaagacggcaagaccagcagcgcgagcaccccggcgacccgtccggttaccggtagctggaaaaagaaccagaagtaccaccatcatcatcatcattaa

SEQ ID NO: 34 - DNA, 1599 bp, synthetic construct
  source 1..1599: mol_type = other DNA
atgcgccgtcgtcgccgccgtcgtcgcggtgctaccaccgttaatgaagccctgaataatgttcgtgcacaggttggtagcggtgttagcgttggtaatggtgaatgttatgcactggcaagctggtatgaacgtatgattagtccggatgcaaccgttggtctgggtgccggtgttggttgggttagcggtgcaattggtgataccattagcgcaaaaaacattggcagcagctataattggcaggcaaatggttggaccgttagcaccagcggtccgtttaaagcaggtcagattgttaccctgggtgcaacaccgggtaatccgtatggtcatgttgttattgttgaagccgttgatggtgatcgtctgaccattctggaacagaattatggtggtaaacgttatccggtgcgtaactattattcagcagcaagctatcgtcagcaggttgttcattatatcacccctccgggtacagttgcacagagcgcaccgaatctggcaggtgcaggcggcaaacatctggtgatttgtggtcatggtcagggtcgtaccggttatgatccgggtgcagttaatgcaaaactgggtattaccgaagcaggtaaagttcgtgaactgagcaaactgatgagcaaatatagcggtcagcagatcgattttatcaccgaacagaacgtgtatgattatcgtagcattaccagcattggtaaaggctatgacagcattaccgaactgcattttaatgcctttaatggtagcgcaaaaggcaccgaagttctgattcagagcagcctggaagcagataaagaagatatggcaattctgagcctgctgagccgttattttcagaatcgtggtatcaaaaaagtggattggctgtataatgcaaatcaggcagcaagccgtggttatacctatcgtctggttgaaattgccttcatcgataacgaacaggatatggccatctttgaaaacaaaaaagaggatattgccaaaggtctggttagcgcaattaccggtgttgaagttaaaaccattgttccgagccctccgagcagcaccgttggtagcagcggcaccccgagcaaaagcatttatctggttggtgatagcctgcgtgttctgccgcatgcaacccattatcagaccggtcagaaaattgcaaattgggttaaaggtcgcacctacaaaattctgcaggttaaaaatgtgcatcagagcaatagcaaacgtgcatatctgctggatggtattaaaagctgggttctggaacaggatgttgaaggcaccaccaaaggtcatagcggaagcagccgtagctatcgtgaaaccggcaccatgaccgttaccgttgatgcactgaatgttcgtcgtgcaccgaataccagcggtgaaattgttgcagtgtataaacgtggtgagagcttcgattatgataccgtgattattgatgtgaacggttatgtttgggtgagctatattggtggttcaggcaaacgtaattatgttgcaaccggtgccaccaaagatggtaaacgctttggtaatgcatggggcacctttaaaaagtaccaccatcatcatcatcattaa

SEQ ID NO: 35 - protein, 9 aa, synthetic construct
MRRRRRRRR

SEQ ID NO: 36 - protein, 12 aa, synthetic construct
MYGRKKRRQRRR

SEQ ID NO: 37 - protein, 16 aa, synthetic construct
MRQIKWFQNRRMKWKK

SEQ ID NO: 38 - protein, 22 aa, synthetic construct
MKETWWETWWTEWSQPKKKRKV

SEQ ID NO: 39 - protein, 41 aa, synthetic construct
MGFGCNGPWNEDDLRCHNHCKSIKGYKGGYCAKGGFVCKCY

SEQ ID NO: 40 - protein, 41 aa, synthetic construct
MGFGCNGPWNEDDLRCKNHCKSIKGYKGGYCAKGGFVCKCY

SEQ ID NO: 41 - protein, 41 aa, synthetic construct
MGFGCNGPWNEDDLRCRNHCKSIKGYKGGYCAKGGFVCKCY

SEQ ID NO: 42 - protein, 41 aa, synthetic construct
MGFGCNGPWNEDDLRCHNKCKSIKGYKGGYCAKGGFVCKCY

SEQ ID NO: 43 - protein, 41 aa, synthetic construct
MGFGCNGPWNEDDLRCHNRCKSIKGYKGGYCAKGGFVCKCY

SEQ ID NO: 44 - protein, 41 aa, synthetic construct
MGFGCNGPWNEDDLRCKNKCKSIKGYKGGYCAKGGFVCKCY

SEQ ID NO: 45 - protein, 41 aa, synthetic construct
MGFGCNGPWNEDDLRCKNRCKSIKGYKGGYCAKGGFVCKCY

SEQ ID NO: 46 - protein, 41 aa, synthetic construct
MGFGCNGPWNEDDLRCRNKCKSIKGYKGGYCAKGGFVCKCY

SEQ ID NO: 47 - protein, 41 aa, synthetic construct
MGFGCNGPWNEDDLRCRNRCKSIKGYKGGYCAKGGFVCKCY

SEQ ID NO: 48 - protein, 42 aa, synthetic construct
PSGNIPKTPSDAPKSEVVNSSTEPKTEETGANGKATDTKITK

SEQ ID NO: 49 - protein, 14 aa, synthetic construct
PPGTVAQSAPNLAG

SEQ ID NO: 50 - protein, 4 aa, synthetic construct
KGHS

SEQ ID NO: 51 - protein, 14 aa, synthetic construct
MYGRKKRRQRRRGA

(19) United States
(12) Patent Application Publication
(10) Pub. No.: US 2026/0275317 A1
(43) Pub. Date: Sep. 17, 2026

(54) CHIMERIC ENDOLYSINS WITH ACTIVITY AGAINST STREPTOCOCCI AND STAPHYLOCOCCI

(71) Applicants: UNIVERSITEIT GENT, Gent (BE); KATHOLIEKE UNIVERSITEIT LEUVEN, Leuven (BE)

(72) Inventors: Niels Vander Elst, Merelbeke (BE); Yves BRIERS, Gent (BE); Evelyne MEYER, Merelbeke (BE); Rob LAVIGNE, Leuven (BE)

(21) Appl. No.: 19/469,202

(22) PCT Filed: Mar. 28, 2024
(86) PCT No.: PCT/EP2024/058545

§ 371 Date: Sep. 25, 2025

(30) Foreign Application Priority Data
Mar. 29, 2023 (EP) 23164960.9
Oct. 24, 2023 (EP) 23205478.3

(51) Int. Cl. C12N 9/36; A61K 31/431; A61K 38/00; A61P 31/04; C07K 14/00

(52) U.S. Cl. CPC C12N 9/2462; A61K 31/431; A61P 31/04; C07K 14/00; C12Y 302/01017; A61K 38/00; C07K 2319/70

(57) ABSTRACT

The present invention relates to novel fusion proteins, more specifically chimeric endolysins, and compositions comprising them. Furthermore, the invention relates to polynucleotides, such as nucleic acids and vectors encoding for said chimeric endolysins, as well as host cells comprising these polynucleotides. Also provided by the present invention is a pharmaceutical composition comprising an endolysin of the invention. Finally, the present invention relates to chimeric endolysins, polynucleotides (such as nucleic acid sequences, vectors), host cells, or compositions for use in medicine, in particular veterinary medicine, more in particular for the treatment and/or prevention of infections, in particular mammary gland infections, more in particular udder infections caused by Streptococci and/or Staphylococci.

### FIELD OF THE INVENTION

The present invention relates to novel fusion proteins, more specifically chimeric endolysins and compositions comprising them. Furthermore, the invention relates to polynucleotides, such as nucleic acids and vectors encoding for said chimeric endolysins, as well as host cells comprising these polynucleotides. Also provided by the present invention is a pharmaceutical composition comprising an endolysin of the invention. Finally, the present invention relates to chimeric endolysins, polynucleotides (such as nucleic acid sequences, vectors), host cells, or compositions for use in medicine, in particular veterinary medicine, more in particular for the treatment and/or prevention of infections such as mammary gland or udder infections caused by Streptococci and/or Staphylococci.

### BACKGROUND TO THE INVENTION

Bovine mastitis, an infection of the cow's udder, is the most important economic disease affecting dairy cattle. Staphylococcus aureus, Streptococcus uberis, Streptococcus dysgalactiae, and to a lesser extent Streptococcus agalactiae, are considered a substantial cause of this disease.

Streptococcal mastitis is usually treated with intramammary antibiotics, but the use of these antibiotics has several disadvantages. First of all, antibiotics that are considered critical for human health care by the world's health organization (WHO) and European Union (EU) are still frequently applied although their use is prohibited by law (e.g. cefquinome, a fourth-generation cephalosporin). Dairy farmers currently lack alternatives for these superior antimicrobials, resulting in their persistent use. The latter fact is a major concern in the transition to a more sustainable way of producing dairy products, in which the use of critical antibiotics has no longer a place. Dairy cows are usually treated with antibiotics at the end of their lactation period to counteract infection in their dry period. Indeed, dairy farmers apply antibiotics in a preventive way leading to a consistent overuse in the sector. Moreover, bovine staphylococci and streptococci penetrate bovine mammary epithelial cells and are able to form biofilms, which are regarded as virulence factors that allow them to circumvent antibiotic therapy. This causes the infection to chronically persist and reoccur after antibiotic treatment. Furthermore, the emergence of antimicrobial resistance (AMR) is also a concern in veterinary medicine and the development of new pharmaceuticals with required activity in raw cow's milk is an additional hurdle for companies to proceed with their compounds to a bovine mastitis application.

To circumvent these disadvantages/limitations of antibiotics, several alternatives such as probiotics and vaccination (e.g. UBAC marketed by HIPRA) have been proposed but till date did not succeed to provide the highly anticipated curative and/or preventive therapeutic relief. This is of particular importance for S. aureus, S. uberis and S. dysgalactiae, as these pathogens propagate both in the cow's environment and in the infected cow's udder.

In contrast, bacteriophage-derived endolysins (enzyme-based antibiotics or “enzy-biotics”) are increasingly gaining attention due to their specificity, high killing efficacy, unlikely resistance development and rapid degradation in the environment. Indeed, these lytic enzymes from a Gram-positive background feature a modular design, consisting of enzymatically active domains (so called EADs), which define the enzyme's cleavage sites, and cell wall-binding domains (so called CBDs), which confer their bacterial specificity. Although several research groups have evaluated natural and/or rationally designed endolysins in the context of bovine Streptococcal mastitis (i.e., λSA2 & B30 (Schmelcher et al. 2015), Ply0643 (Liu G et al. 2021), PlyC (Nelson D et al. 2006), ClyR (Yang H et al. 2015, PlySs2 &-9 (Vander Elst N et al. 2020)), the primary focus of the scientific community has mainly been on St. aureus (i.e., LysRODI (Gutiérrez D et al. 2020), TRX-SA1 (Fan J et al. 2016), phi11 Donovan D M et al. 2006), φH5 (Obeso J M et al. 2008) & Lys109 (Son B et al. 2021)).

On the other hand, bacteriophages featured by self-replication, have also been proposed and investigated as an innovative strategy to combat bovine mastitis, albeit mainly in the context of Staphylococcus aureus. However, the use of bacteriophages has several disadvantages compared to (engineered) endolysins: high specificity at the strain level requiring personalized medicine or phage cocktails, resistance development on multiple levels, higher chance on developing neutralizing anti-drug antibodies, transfer of unknown and potentially harmful genes, and non-predictable pharmacokinetics and -dynamics.

To counter the rise of drug-resistant pathogenic bacteria, there is an urgent and unmet need for new treatment strategies. High-throughput screening of chimeric endolysins to simultaneously target Staphylococcus aureus and several streptococci (i.e., S. uberis, S. dysgalactiae and/or S. agalactiae), also tackling their virulence factors to circumvent antibiotic therapy (i.e., AMR, biofilm formation and intracellular persistence) and/or combined with significant synergistic activity with antibiotics, defines the value of the engineered endolysins of the present invention.

Accordingly, it was an object of the present invention to provide a solution to some of the problems listed herein above. The inventors have now surprisingly found that the chimeric endolysins according to the invention display improved lytic activity against S. aureus, S. uberis, S. dysgalactiae and/or S. agalactiae peptidoglycan, and living cells of the bacterial species S. uberis, S. dysgalactiae, and/or S. agalactiae. Moreover, the endolysins according to the invention are particularly suitable as an add-on therapy to the current antimicrobial arsenal (antibiotics) against streptococci or staphylococci.

Main advantages of the present invention are that the use of chimeric endolysins e.g. in veterinary medicine (be it as an add-on therapy with antibiotics) leads to (1) less spreading of the infection in the herd, which in its own way will translate into less therapeutic use of antibiotics and less premature slaughtering of affected animals; (2) a decrease in the therapeutic dose of antibiotics used in animal agriculture as advocated by the WHO, EU, as well as the dairy consumer; (3) an increase for the dairy farmer of his/her profitability, as a more effective treatment will lead to less veterinary costs, less chronically infected animals and thus an increase in milk production; (4) animal welfare due to an improved treatment.

### SUMMARY OF THE INVENTION

In a first aspect, the present invention provides a fusion protein, comprising
a cell penetrating peptide (CPP);
at least one enzymatic activity domain (EAD); and
at least one cell wall binding domain (CBD) comprising an amino acid sequence selected from SEQ ID NO: [1] (i.e. Ply1081 CW_7), SEQ ID NO: [2] (i.e. PlySs2 SH3_5), or a sequence having at least 85% identity thereto.

In a further embodiment, the present invention provides a fusion protein wherein said at least one EAD comprises an amino acid sequence as set forth in SEQ ID NO: [3] (i.e. PlySs9 Amidase3), or a sequence having at least 85% identity thereto.

In another embodiment, the present invention provides a fusion protein further comprising at least one other EAD comprising an amino acid sequence as set forth in SEQ ID NO: [4] (i.e. PlySs2 CHAP), or a sequence having at least 85% identity thereto.

In a specific embodiment, the present invention provides a fusion protein wherein said at least one CBD comprises an amino acid sequence as set forth in SEQ ID NO: [1], or a sequence having at least 85% identity thereto.

In a particular embodiment, a fusion protein is provided wherein said CPP is located at the N-terminal position of said fusion protein.

In another particular embodiment, the fusion protein according to the invention comprises the peptide and domains in the order CPP-EAD-CBD-EAD from the N-terminal to the C-terminal position.

In yet a specific embodiment, the present invention provides a fusion protein wherein said CPP is positively charged.

In another embodiment, the present invention provides a fusion wherein said CPP is selected from the list comprising: HIV-1 TAT, polyarginine, NZ2114 or any NZ2114-derived peptide, Pep-1 and Penetratin.

In a specific embodiment, the fusion protein according to the invention comprises an amino acid sequence as set forth in SEQ ID NO: [17] (i.e. NC5), or a sequence having at least 85% identity thereto.

In a specific embodiment, the fusion protein according to the invention comprises an amino acid sequence as set forth in SEQ ID NO: [15] (i.e. NC3), or a sequence having at least 85% identity thereto.

In a specific embodiment, the fusion protein according to the invention comprises an amino acid sequence as set forth in SEQ ID NO: [20] (i.e. NC8), or a sequence having at least 85% identity thereto.

In a specific embodiment, the fusion protein according to the invention comprises an amino acid sequence as set forth in SEQ ID NO: [14] (i.e. NC2), or a sequence having at least 85% identity thereto.

In a further aspect, an isolated nucleic acid encoding the fusion protein according to the invention is provided.

In a specific embodiment, the present invention provides an isolated nucleic acid wherein said nucleic acid comprises a nucleic acid sequence that has at least 75% identity as set forth in SEQ ID NO: [29].

In yet a further aspect, the present invention provides a vector comprising the nucleic acid according to the invention.

In a further embodiment, the present invention provides a host cell expressing the fusion protein according to the invention, or a host cell comprising the nucleic acid or the vector according to the invention.

The invention furthermore encompasses a method of making the fusion protein provided herein, said method comprising the steps of:
introducing into a host cell a nucleic acid or construct encoding the fusion protein;
culturing said host cell under conditions suitable for expression of said protein; and
recovering the protein so expressed.

In yet a further aspect, the present invention provides a pharmaceutical composition or combination comprising the fusion protein, the nucleic acid, the vector, or the host cell according to the invention, and a pharmaceutically acceptable excipient.

In a particular embodiment, the pharmaceutical composition or combination according to the invention, further comprising at least one antibiotic, in particular a beta-lactam antibiotic, more in particular penicillin, even more in particular cloxacillin.

In a further aspect, the present invention provides a fusion protein, a nucleic acid, a vector, a host cell, or a pharmaceutical composition or combination according to the invention, for use in medicine, in particular for use in veterinary medicine.

In another embodiment, the present invention provides a fusion protein, composition or combination for use in the treatment and/or prevention of an infection caused by Staphylococcus aureus, Streptococcus uberis, Streptococcus dysgalactiae and/or Streptococcus agalactiae in a subject, in particular by Streptococcus uberis, Streptococcus dysgalactiae and/or Streptococcus agalactiae.

In a particular embodiment, the present invention provides a fusion protein, composition or combination for use in the treatment and/or prevention of mastitis, in particular bovine mastitis.

In yet another embodiment, the present invention provides a fusion protein, composition or combination for use in the treatment and/or prevention of an infection, wherein the infection is of (part of) the mammary gland, in particular of the udder, more in particular the bovine udder.

In a specific embodiment, the present invention provides a fusion protein, composition or combination for use in the treatment and/or prevention of an infection, wherein the subject is a (mono- or polygastric) mammal, in particular a cow, buffalo, goat, sheep, camel, yak, horse, reindeer or donkey, in particular a ruminant, more in particular a (dairy) cow.

In a very specific embodiment, the fusion protein, composition or combination according to the invention is administered locally, in particular intraductally such as through (one of) the teat orifice(s) via the teat canal(s) of the mammary gland/udder, or on the teat apex such as through dipping.

In a further aspect, the present invention provides a method of treating and/or preventing an infection or disease in a subject comprising administering to said subject an effective dosage of a fusion protein, a nucleic acid, a vector, a host cell, a pharmaceutical composition or combination according to the invention.

In a further embodiment of the method of the present invention, the infection is caused by Staphylococcus aureus, Streptococcus uberis, Streptococcus dysgalactiae and/or Streptococcus agalactiae, in particular by Streptococcus uberis, Streptococcus dysgalactiae and/or Streptococcus agalactiae.

### BRIEF DESCRIPTION OF THE DRAWINGS

With specific reference to the figures, it is to be noted that the particulars shown are by way of example and for purposes of illustrative discussion of the different embodiments of the present invention. They are presented in the cause of providing what is believed to be the most useful and readily description of the principles and conceptual aspects of the invention. In this regard no attempt is made to show structural details of the invention in more detail than is necessary for a fundamental understanding of the invention. The description taken with the drawings make it apparent to those skilled in the art how the several forms of the invention may be embodied in practice.

FIG. 1: General overview of the high throughput assembly, screening and hit-to-lead selection of 6 libraries (A to F) containing 88,704 designer endolysins. The modular subdomains of the designer endolysins are represented as follows: cell penetrating peptides (CPPs) as circles, enzymatically active domains (EADs) as squares and cell wall binding domains (CBDs) as triangles.

FIG. 2: Graphic display of six designs selected to screen for bispecific designer endolysins, together with the number of theoretical combinations possible per design. In total, 88,704 theoretical combinations can be VersaTile-assembled. CPP, EAD, CBD indicates the position in each design, whereas the polyhistidine tag (HIS) is included for purification purposes.

FIG. 3: Transformed E. coli BL21 (pLysS). Transformed E. coli BL21 (pLysS) cells with one colony expressing a designer endolysin with activity against autoclaved S. aureus N305 cells (A). This colony was subsequently picked and streaked out on the same agar, containing autoclaved S. uberis 0140J cells instead (B). If clearing zones (i.e., “halos”) were observed against the peptidoglycan of both pathogens, the designer endolysin was regarded a bispecific hit.

FIG. 4: Graphic display of the twelve chimeric endolysins withheld after screening of the VersaTile-assembled libraries with in vitro lytic activity against S. uberis 0140J and S. aureus N305 peptidoglycan. The corresponding molecular weight (MW) and isoelectric point (pI) of each chimeric endolysin is shown. The modular subdomains of the designer endolysins are represented as follows: the N-terminal CPP, EADs, CBDs and the C-terminal HIS have white, light grey, dark grey and black fillings, respectively.

FIG. 5: Relative in vitro scoring system of eleven of the twelve identified chimeric endolysins in PBS and UHT-milk against the bovine mastitis pathogens S. uberis, S. aureus, S. agalactiae and S. dysgalactiae. A cumulative score of 3.0 corresponds to a candidate that obtained the maximum score in each of the individual conditions performed. (A) S. uberis, (B) S. aureus, (C) S. agalactiae (D) S. dysgalactiae. (E) Cumulative in vitro score for each chimeric endolysin against all 4 pathogens. NC4 could not be included due to instability in PBS.

FIG. 6: Comparative turbidity reduction assay performed with a 0.5 μM concentration of 3 chimeric endolysins against stationary phase S. uberis 0140J. NC3 (square), NC5 (triangle) and NC8 (diamond). Bacteria in PBS (circle) served as negative control.

FIG. 7: Turbidity reduction (A) and time killing (B) assays with a 0.3 μM concentration of 2 chimeric endolysins against stationary phase S. uberis 0140J. NC2 (open diamond) and NC5 (triangle). Bacteria in PBS served as negative controls (round dots). Δ indicates the observed difference in log₁₀ (CFU/mL), *** indicates p<0.001.

FIG. 8: Turbidity reduction (A, B, D, E) and time killing (C, F) assays with a 0.3 μM (black triangles) and a 0.9 UM (white triangles) concentration of chimeric endolysin NC5 against bovine mastitis-isolated stationary phase S. uberis belonging to the global clonal complex (GCC ST-) 5 & 143 (A, B & C), and S. agalactiae & dysgalactiae (D, E & F). Bacteria in PBS served as negative controls (black dots). Δ indicates the observed difference in log₁₀ (CFU/mL), *** indicates p<0.001.

FIG. 9. Time killing assays with a concentration of 0.3 μM of 2 natural endolysins versus NC5 against bovine mastitis-isolated stationary phase S. uberis GCC ST-5 (A), S. agalactiae (B) and S. dysgalactiae (C). PlySs2 (right filled square), PlySs9 (left filled square) and NC5 (black triangles). Bacteria in PBS served as negative controls (black dots). Δ indicates the observed difference in log₁₀ (CFU/mL), * and *** indicate p<0.05 and p<0.001, whereas ‘ns’ indicates non-significance corresponding to a p >0.05.

FIG. 10: Biofilm mass of bovine mastitis pathogen S. uberis. (A) Determination of the biofilm composition of S. uberis GCC ST-143 by proteinase K, DNase I and NaIO₄ treatments, as well as eradications of biofilm (B) biomass and (C) CFU by a concentration of 1.5 μM NC5 after incubation during 2 h30 at 37° C. on a shaker. Δ indicates the observed difference in log₁₀ (CFU/mL), whereas *, ** and ‘ns’ indicate p<0.05, p<0.001 and non-significance corresponding to a p >0.05, respectively.

FIG. 11: Intracellular killing of S. uberis GCC ST-5 in the bovine mammary epithelial cell lines (A) MAC-T and (B) PS after challenging infected cells with a concentration of 2.5 μM NC5 at 37° C. during 1 h. MAC-T and PS were infected at a multiplicity of infection of approximately 250 and 500, respectively. Cell culture medium without the addition of NC5 served as the negative control (0.0 μM). Δ indicates the observed difference in log₁₀ (CFU/mL), whereas ** and *** indicate p<0.01 and p<0.001, respectively.

FIG. 12: Challenge of mastitic raw cow's milk from 4 infected animals (A, B, C, D) during 8 h with either a concentration of 0.5 μM NC5 or 50 μg/mL of the reference treatment beta-lactam penicillin antibiotic or a combination of both. NC5 (triangle), cloxacillin (diamond), a combination of both (square), PBS (circle) as a negative control. The amount of surviving bacteria was determined respecting a 2 h interval. Δ indicates the observed difference in log₁₀ (CFU/mL) observed between the combination treatment and the negative control, whereas the difference between the combination treatment and the 50 μg/mL cloxacillin group is indicated by the p-value or *, ** and *** corresponding to a p<0.05, p<0.01 and p<0.001, respectively.

FIG. 13. Comparative evaluation of the hallmarks of mastitis in S. uberis-infected murine mammary glands treated with endolysin NC5 in either a low or a high dose combined with cloxacillin, in comparison to cloxacillin stand-alone and placebo (PBS) treatment. (A) Bacterial load with or (B) without presumed slow responding mice, and (C) Ly6G-positive staining for neutrophil quantification in mammary glands of presumed fast responding mice, all harvested 4 h after intraductally treating S. uberis-infected murine mammary glands. Data are shown as individual points representing each mouse with a bar indicating the mean and an error bar representing the standard error of the mean (SEM). The dotted line indicates the mean±2×SEM of the placebo group as a criterium to exclude presumed slow responding mice. LOD: limit of detection (400 CFU/g tissue).

FIG. 14. Comparative evaluation of the inflammatory protein profile between the different treatment groups of this study. Mice that showed a slow response in bacterial load were excluded for the mediators G-CSF and LCN2. (A) MIP-2, (B) G-CSF, (C) LCN2, (D) IL-1a, (E) IL-1B, (F) IL-6, (G) TNF-α, (H) MCP-1, (I) M-CSF and (J) CHI3L1 quantification of mouse mammary glands harvested 4 h after intramammary treatment with either placebo (PBS), 30 μg cloxacillin, or a combination of 30 μg cloxacillin with either a low (23.5 μg) or high (235.0 μg) dose of endolysin NC5. Data are shown as individual points representing each mouse with a bar indicating the mean and an error bar representing the standard error of the mean. The following outliers were removed: 822.0 μg/mL for IL-6 in the cloxacillin stand-alone group; 6.6 μg/mL for LCN2 in the low dose combination therapy group; 7,574.3 pg/mL for G-CSF, 263.6 and 520.1 μg/mL for IL-6, 189.7 and 223.0 μg/mL for MCP-1 and 8.9 μg/mL for M-CSF, all in the high dose combination therapy group. Single (*) and double (**) asterisks indicate p<0.05 and p<0.01, respectively.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

As used in the specification and the appended claims, the singular forms “a”, “an”, and “the” include plural referents unless the context clearly dictates otherwise. By way of example, “a compound” means either one compound or more than one compound.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Throughout the description and claims of this specification, the word “comprise” and other forms of the word, such as “comprising” and “comprises,” means including but not limited to, and is not intended to exclude, for example, other additives, components, integers, or steps. The term “consisting essentially of” or “consists essentially of” means that e.g. a product or method must contain the listed compounds, ingredient(s), or steps and may also contain small amounts (for example up to 5% by weight, or up to 1% or 0.1% by weight) of other ingredient(s), compounds, or steps provided that any additional ingredients, compounds, or steps do not affect the essential properties of the respective product or method. The terms described above and others used in the specification are well understood to those in the art. All references, and teachings specifically referred to, cited in the present specification are hereby incorporated by reference in their entirety.

Several optimized chimeric endolysins tackling different streptococcal species causing e.g. infections of the mammary gland have been developed in the present invention employing a high-throughput assembly method combined with relevant in vitro and in vivo screening tools.

The overall objective of the present invention is to provide an engineered and/or recombinant endolysin displaying improved lytic activity against streptococci such as for example S. uberis, S. dysgalactiae, and/or S. agalactiae, and/or against staphylococci such as S. aureus. Moreover, the endolysins according to the invention are particularly suitable as an add-on therapy to the current antimicrobial arsenal against infections caused by streptococci, in particular against infections of the mammary gland, more in particular against bovine mastitis-causing streptococci and/or staphylococci.

The definitions and preferred aspects defined herein above and below for the endolysins of the present invention apply, mutatis mutandis also for the nucleic acid molecule, vector, host cell, pharmaceutical composition, combinations, uses and method of treatment of the present invention.

### Definitions

The term “lysins” refers to cell-wall lytic enzymes encoded by bacteriophages (endolysins) or bacteria (autolysins) which have the ability to hydrolyse the cell-wall of target bacteria when added exogenously. Endolysins have important advantages over classical antibiotics, e.g. a novel mode of action; a narrow spectrum of susceptible bacteria; rapid killing of both stationary- and exponentially-growing bacteria; activity on mucous membranes and bacterial biofilms; low probability of developing resistances; and reduced impact on normal microbiota. Lysins from Gram-positive bacteria and their phages usually comprise at least one catalytic domain and one or more cell wall-binding domains. In contrast, many lysins produced by Gram-negative species or their phages only contain the catalytic domain, though modular endolysins have also been reported. The catalytic units dictate the type of peptidoglycan (PG) bond to be cleaved, whereas the cell wall-binding domain(s) largely determines the lytic spectrum by specific recognition of cell wall elements distributed in genus-, or species/strain-specific manner. Endolysins show a modular organization exhibiting a combination of different polypeptide domains showing enzymatic activity or cell binding activity, the so-called “EADs” (enzymatically active domains) and “CBDs” (cell binding domains), respectively. Mostly, EADs are located at the N-terminal part of the endolysins, and CBDs at the C-terminal parts, but there are also exceptions to this.

In the context of the present invention, the term “natural endolysin” or “wild-type endolysin” refers to an endolysin encoded by a prophage sequence within a bacterial genome. In the context of the present disclosure, the term “natural endolysin” therefore refers to an endolysin which has not been domain-swapped. A natural endolysin can be unmodified, meaning that the amino acid sequence of the endolysin corresponds to the native sequence. Examples of natural endolysins are PlySs2 and PlySs9, respectively derived from Streptococcus suis serotype-2 and -9 prophages (Vander Elst et al., 2020).

In the context of the present invention, the term “chimeric endolysin”, “recombinant endolysin” or “engineered endolysin” refers to an (artificial) endolysin having domains of different origin and/or in an assembly that is different than the natural endolysin. A chimeric endolysin can be unmodified, meaning that the amino acid sequence of the endolysin corresponds to the native sequence of the respective domains composing the endolysin. Alternatively, a chimeric endolysin can be modified, meaning that the amino acid sequence of the endolysin comprises at least one mutation compared to the native sequence of the respective domains composing the endolysin. In line with this definition, the person skilled in the art readily understands that the chimeric endolysins as described herein are non-naturally occurring endolysins. That is, the chimeric endolysin of the present invention has been modified by hand of man and excludes, by definition, natural endolysins, i.e., as it can be naturally found in nature. The examples herein provide suitable method(s) how to generate the chimeric endolysin of the invention. In the present invention, chimeric, recombinant or engineered endolysins are also referred to as “fusion proteins”, resulting from the joining or fusion of different domains/(poly) peptides in one protein.

The person skilled in the art will further understand that the chimeric, recombinant or engineered endolysins as described can be synthetically produced or expressed in an expression system which is not the native expression system e.g. a recombinant expression system such as a recombinant vector. In a particular embodiment, a chimeric endolysin or protein can thus be interpreted as an endolysin or protein expressed in an expression system which is different from the expression system it is natively expressed in. The term “recombinant” also includes molecules formed by laboratory methods of genetic recombination, which bring together genetic material from different sources, thereby creating sequences which would not otherwise be found in the native genome.

The term “isolated” is used to indicate that a cell, protein, (poly) peptide or nucleic acid (polynucleotide) is separated from its native environment. Isolated cells, proteins, (poly) peptides and nucleic acids may be substantially pure, i.e., essentially free of other substances with which they may bound in nature.

The terms “catalytic domain” or “enzymatic domain” refer to the part of the protein chain which contains the region where the catalysed chemical reaction takes place.

As used herein, “cell-penetrating peptides” (CPPs), also known as protein transduction domains (PTDs), are short peptides (up to 25, 30, 35 or 40 residues) that are not only able to translocate small macromolecular drugs, nucleic acids, proteins, viruses, or imaging agents across plasma membranes but also allow CPP/cargo complexes to transport across the cell membrane, by different endocytosis pathways depending on the types of CPPs. Hence, CPPs with membrane penetrating function could transport hydrophilic macromolecules to eukaryotic cells through energy-independent pathways. CPPs typically have an amino acid composition that contains either a high relative abundance of positively charged basic amino acids such as lysine or arginine, or an alternating pattern of polar, positively charged amino acids and non-polar, hydrophobic amino acids. These two types of structures are referred to as polycationic or amphipathic, respectively. A third class of CPPs are the hydrophobic peptides, containing either only apolar residues with low net charge or hydrophobic amino acid groups that are crucial for cellular uptake.

The terms “peptide”, “polypeptide”, “protein” and variations of these terms refer to peptide, oligopeptide, oligomer or protein, including a fusion protein, respectively, comprising at least two amino acids joined to each other by a normal or modified peptide bond, such as in the cases of the isosteric peptides, for example. These terms also include herewith “peptidomimetics” which are defined as peptide analogs containing non-peptidic structural elements, which peptides are capable of mimicking or antagonizing the biological action(s) of a natural parent peptide.

The term “variant” refers to a protein, (poly) peptide or nucleic acid including one or more insertions, deletions, and/or substitutions, either non-conservative or preferably conservative, relative to the native (wild-type) amino acid or nucleic acid sequence. More particular, said one or more amino acid substitution is a ‘conservative’ amino acid substitution, i.e., the substitution of an amino acid by another amino acid of the same class, in which the classes are as follows:

| Class | Amino acid examples |
|---|---|
| Nonpolar | Ala, Val, Leu, Pro, Met, Phe, Trp, Ile |
| Uncharged polar | Gly, Ser, Thr, Cys, Tyr, Asn, Gln |
| Acidic | Asp, Glu |
| Basic | Lys, Arg, His |

In one embodiment, a protein or (poly) peptide may comprise an amino acid sequence with at least 85% identity to the reference or wild-type amino acid sequence, preferably at least 90% identity, more preferably at least 95% identity, even more preferably at least 96% identity, even more preferably at least 97% identity, even more preferably at least 98% identity, even more preferably at least 99% identity, even more preferably at least 99.5% identity to a reference or wild-type amino acid sequence. In another embodiment, a nucleic acid may comprise an nucleic acid sequence with at least 85% identity to the reference or wild-type nucleic acid sequence, preferably at least 90% identity, more preferably at least 95% identity, even more preferably at least 96% identity, even more preferably at least 97% identity, even more preferably at least 98% identity, even more preferably at least 99% identity, even more preferably at least 99.5% identity to a reference or wild-type nucleic acid sequence. The percentage identity of nucleic acid and protein or (poly) peptide sequences can be calculated using commercially available algorithms, which compare a reference sequence with a query sequence. The following programs (provided by the National Center for Biotechnology Information) may be used to determine homologies/identities: BLAST, gapped BLAST, BLASTN and PSI BLAST, which may be used with default parameters. Preferably, % identity is identified over the whole lengths of the sequences to be compared. It will be appreciated that percent identity is calculated in relation to polypeptides or nucleic acids whose sequence has been aligned optimally. Fragment and variants of an amino acid sequence may be made using any of the methods of protein engineering, directed evolution and/or site-directed mutagenesis well known in the art (for example, see Molecular Cloning: a Laboratory Manual, 3rd edition, Sambrook & Russell, 2001, Cold Spring Harbor Laboratory Press). It will be appreciated by a skilled person that a polypeptide according to the invention, or fragment, variant, or fusion thereof, may comprise or consist of a derivative of a native amino acid sequence, or a fragment or variant thereof. Chemical derivatives of one or more amino acids may be achieved by reaction with a functional side group. Derivatives also include proteins or peptides containing one or more additions or deletions as long as the requisite activity is maintained.

As used herewith “bacterial infections and disorders” refer to infections and disorders caused by bacteria. Of particular interest in the context of the invention are infections and disorders caused by at least one strain of the Streptococcus genus, in particular selected from the group comprising Streptococcus uberis, Streptococcus dysgalactiae, and/or Streptococcus agalactiae. In a further embodiment, the chimeric endolysins of the invention have also been found biochemically active against Staphylococcus aureus peptidoglycan. Therefore, the endolysins according to the invention may be effective in a dose-dependent way for infections caused by S. aureus. In a particular embodiment, the chimeric endolysin according to the invention is a bispecific chimeric endolysin which acts against the peptidoglycans of both pathogens. Bacterial infections and disorders include infections of the mammary gland, in particular of the breast, udder or dugs, such as mammitis or mastitis. A mammary gland is an exocrine gland in humans and other mammals that produces milk to feed young offspring. The mammary glands are arranged in organs such as the breasts in primates (for example, humans and chimpanzees), the udder in ruminants (for example, cows, goats, sheep, and deer), and the dugs of other animals (for example, dogs and cats).

As defined herewith the terms “killing activity” of an endolysin against a particular bacterium represents a reduction in the number of viable bacterial cells caused by the lysing activity of said endolysin. The killing activity of the endolysin against said bacteria can be complete, meaning that 100% of the bacterial cells have been lysed, or partial meaning that at least about 60%, at least about 70%, at least about 80%, at least about 90%, or at least about 95% of the bacterial cells have been lysed. Killing activity can be determined by measuring a decrease in optical density (OD) at 600-620 nm, in particular OD600, of a bacterial cell suspension and/or a decrease in Colony Forming Units (CFU) per millilitre (mL) of a bacterial cell suspension after exposure to the endolysin to be tested.

As defined herewith the terms “binding capacity” of an endolysin to the cell wall of a particular bacterium refers to the ability of said endolysin to specifically interact and adhere to the cell wall of said bacterium. The binding capacity of an endolysin to the cell wall of bacteria can be determined by methods known in the art.

As used herein, “treatment” and “treating” and the like generally mean obtaining a desired pharmacological and physiological effect. The effect may be therapeutic in terms of a partial or complete cure of a disease, condition, symptom or adverse effect attributed to the disease. The term “treatment” as used herein covers any treatment of a disease in a mammal as provided and includes: (a) inhibiting the disease, i.e., arresting its development; or relieving the disease, i.e., causing regression of the disease and/or its symptoms or conditions such as improvement or remediation of damage. In particular, treatment of bacterial infections comprises decreasing or even eradicating the infection, for instance by killing the bacteria and, thus, controlling, reducing or inhibiting bacterial proliferation as well as reducing the number of viable bacterial cells. Herein it is preferred that the disease, e.g. mastitis or infection of the mammary gland (e.g. udder) is treated therapeutically in terms of a partial or complete cure of the disease or the symptoms.

As used herein, the term “prevention” or alternatively “to prevent” is to be understood as prophylactically ‘stopping’, ‘averting’, ‘arresting’, ‘blocking’, ‘reducing’ or ‘halting’ disease symptoms, parameters or causal factors that are related with the development of a particular disease, condition, or infection, before the actual onset of the disease, condition, or infection. More specifically, the pharmacological and physiological effects may be prophylactic in terms of preventing or partially preventing a disease, condition or infection from occurring in subjects who have not yet been diagnosed with a disease or who do not (yet) perceive or experience any symptoms related to the disease (i.e., asymptomatic subjects). In particular, prevention of bacterial infections comprises decreasing or even eradicating the developing infection, for instance by killing the bacteria and, thus, controlling, reducing or inhibiting bacterial proliferation as well as reducing the number of viable bacterial cells before symptoms are perceived. The term ‘prevention’ also includes reducing the risk for a subject of getting infected.

The term “effective dosage” as used herein refers to an amount of at least one endolysin according to the invention, composition, combination or pharmaceutical composition thereof, that elicits the biological or medicinal response in a tissue, system, animal or human that is being sought and as provided herein. In one embodiment, the effective dosage is a “therapeutically effective dosage” for the alleviation of the symptoms of the disease or condition being treated. In another embodiment, the effective dosage is a “prophylactically effective dosage” for prophylaxis of the symptoms of the disease or condition being prevented. The term also includes herein the amount of active protein or polypeptide sufficient to reduce the progression of the disease, notably to reduce or inhibit the disorder or infection and thereby elicit the response being sought (i.e., an “inhibition effective dosage”). The relative terms “improved,” “increased,” “enhanced”, “alleviated”, “reduce”, “ameliorate”, “decrease”, “reduce”, and the like refer to the effects of the chimeric endolysin according to the invention or pharmaceutical composition comprising the chimeric endolysin, relative to a composition lacking the chimeric endolysin but otherwise identical (i.e., placebo or control). For example, an endolysin or composition according to the invention can reduce or improve disclosed symptoms by at least 5%, such as at least 10%, at least 15%, at least 20%, preferably at least 25%, more preferably at least 30%, and particularly preferably at least 40%, when compared to untreated subjects. The term “subject” as used herein refers to mammals, in particular female mammals. For examples, mammals contemplated by the present invention include human, primates, domesticated animals such as cattle, sheep, pigs, horses, laboratory rodents and the like. It is preferred that the subject is a (female) ruminant, more in particular cattle, even more in particular a (dairy) cow. The term “ruminants” includes many domesticated animals, or animals that otherwise are of agricultural, veterinary or economic importance, such as sheep, goats, cattle, bison, yaks, water buffalo, deer, camels, llamas, alpacas, as well as various wild animals. “Small ruminants” are understood to include sheep, goats, and deer. The term “cattle” refers to bovine animals including but not limited to steer, bulls, cows, heifers and calves. In another embodiment, the subject is a human.

### Chimeric Endolysins

When reference is made to chimeric endolysin or endolysin according to the invention, one will understand that this refers to endolysins other than natural endolysins. Additionally, one will understand that these terms also refer to the term “fusion protein”. As such, the terms can be used interchangeably. Thus, the chimeric or engineered endolysin is the fusion protein according to the invention resulting from the joining or fusion of different domains/(poly) peptides in one protein.

In a first aspect, the present invention provides a fusion protein, comprising a cell penetrating peptide (CPP), at least one enzymatic activity domain (EAD), and at least one cell wall binding domain (CBD) comprising an amino acid sequence selected from SEQ ID NO: [1] (i.e. Ply1081 CW_7) IDQLVQETLAGKYGNGEQRKAALGSQYQAVMAVINGKATAPKKTVDQLAQEVIQGKHGNGEDRKKSL GPDYDAVQKRVTEILKGS, SEQ ID NO: [2] (i.e. PlySs2 SH3_5): SRSYRETGTMTVTVDALNVRRAPNTSGEIVAVYKRGESFDYDTVIIDVNGYVWVSYIGGSGKRNYVAT GATKDGKRFGNAWGTF, or a sequence having at least 85% identity thereto. In particular for this embodiment, reference is made to FIG. 3.

In a specific embodiment, the isoelectric point (pI) of the fusion protein according to the invention is in the range of 9 to 10, in particular in the range of 9.03 to 9.80, more in particular in the range of 9.05 to 9.65. The “isoelectric point” refers to the pH at which the chimeric endolysin carries no net electrical charge. This is of major relevance for the fusion protein's stability, solubility and lytic activity. More specifically, a pl of the fusion protein in the range 9 to 10, more particular 9.05 to 9.65, assures a positive charge in both normal and mastitic milk (latter having a pH in the range of 6.5 to 7.5), which causes the fusion protein to stay in its soluble form and possess affinity for the negatively charged bacterial cell wall. The pl of a fusion protein is in silico predicted by protein biology software generally known to the skilled person, or as provided in the present examples.

In another embodiment, the molecular weight (MW) of the fusion protein according to the invention is in the range of 20 to 80 kDa, in particular 25 to 70 kDa, more in particular 30 to 65 kDa. The molecular weight of a fusion protein can be in silico predicted by protein biology software and experimentally evaluated by SDS-PAGE and mass spectrometry.

In a specific embodiment, the present invention provides a fusion protein comprising a CPP, at least one EAD, and at least one CBD comprising an amino acid sequence as set forth in SEQ ID NO: [1], or a sequence having at least 85%, 86%, 87%, 88%, 89%, 90%, 91%, 92%, 93%, 94%, 95%, 96%, 97%, 98%, or 99% identity thereto. In particular for this embodiment, reference is made to FIG. 3 and chimeric endolysins NC1, NC3, NC5, NC6, and NC8.

In a further embodiment, the present invention provides a fusion protein comprising a CPP, at least one EAD, and at least one CBD comprising an amino acid sequence as set forth in SEQ ID NO: [2], or a sequence having at least 85%, 86%, 87%, 88%, 89%, 90%, 91%, 92%, 93%, 94%, 95%, 96%, 97%, 98%, or 99% identity thereto. In particular for this embodiment, reference is made to FIG. 3 and chimeric endolysins NA1, NA2, NC2, NC4, NC7, NC9, and E1.

In a further embodiment, the present invention provides a fusion protein comprising a CPP, at least one EAD, and at least one CBD, comprising an amino acid sequence as set forth in SEQ ID NO: [1], or a sequence having at least 85%, 86%, 87%, 88%, 89%, 90%, 91%, 92%, 93%, 94%, 95%, 96%, 97%, 98%, or 99% identity thereto, wherein said at least one EAD comprises an amino acid sequence as set forth in SEQ ID NO: [3] (i.e. PlySs9 amidase_3): GKHLVICGHGQGRTGYDPGAVNAKLGITEAGKVRELSKLMSKYSGQQIDFITEQNVYDYRSITSIGKG YDSITELHFNAFNGSAKGTEVLIQSSLEADKEDMAILSLLSRYFQNRGIKKVDWLYNANQAASRGYTYR LVEIAFIDNEQDMAIFENKKEDIAKGLVSAITGVEVKTIVPSPPSSTVGSSGTPSKSIYLVGDSLRVLPHA THYQTGQKIANWVKGRTYKILQVKNVHQSNSKRAYLLDGIKSWVLEQDVEGTT, or a sequence having at least 85%, 86%, 87%, 88%, 89%, 90%, 91%, 92%, 93%, 94%, 95%, 96%, 97%, 98%, or 99% identity thereto. In particular for this embodiment, reference is made to FIG. 3 and chimeric endolysins NC1, NC3, NC5, NC6, and NC8.

In another embodiment, the present invention provides a fusion protein comprising a CPP, at least one EAD, and at least one CBD, comprising an amino acid sequence as set forth in SEQ ID NO: [1], or a sequence having at least 85%, 86%, 87%, 88%, 89%, 90%, 91%, 92%, 93%, 94%, 95%, 96%, 97%, 98%, or 99% identity thereto, wherein said at least one EAD comprises an amino acid sequence as set forth in SEQ ID NO: [3], or a sequence having at least 85%, 86%, 87%, 88%, 89%, 90%, 91%, 92%, 93%, 94%, 95%, 96%, 97%, 98%, or 99% identity thereto, and wherein said fusion protein comprises at least one further EAD comprising an amino acid sequence as set forth in SEQ ID NO: [2], or a sequence having at least 85%, 86%, 87%, 88%, 89%, 90%, 91%, 92%, 93%, 94%, 95%, 96%, 97%, 98%, or 99% identity thereto. In particular, for this embodiment, reference is made to FIG. 3 and chimeric endolysins NC1, NC3, NC5, NC6, and NC8.

In a very specific embodiment, the fusion protein according to the invention comprises a CPP and an amino acid sequence as set forth in SEQ ID NO: [9], or a sequence having at least 85%, 86%, 87%, 88%, 89%, 90%, 91%, 92%, 93%, 94%, 95%, 96%, 97%, 98%, or 99% identity thereto.

In a specific embodiment, the Ply1081 CW_7 domain is coupled with the PlySs9 Amidase_3 domain, more in particular the Ply1081 CW_7 domain is coupled with the PlySs9 Amidase_3 domain by means of a linker.

In yet a specific embodiment, the present invention provides a fusion protein comprising a CPP, at least one EAD, and at least one CBD comprising an amino acid sequence as set forth in SEQ ID NO: [2], or a sequence having at least 85% identity thereto, wherein at least one EAD comprises an amino acid sequence as set forth in SEQ ID NO: [3], or a sequence having at least 85%, 86%, 87%, 88%, 89%, 90%, 91%, 92%, 93%, 94%, 95%, 96%, 97%, 98%, or 99% identity thereto. In particular for this embodiment, reference is made to FIG. 3 and chimeric endolysins NA2, NC2, NC4, NC7, and E1.

In another embodiment, the present invention provides a fusion protein comprising a CPP, at least one EAD, and at least one CBD comprising an amino acid sequence as set forth in SEQ ID NO: [2], or a sequence having at least 85%, 86%, 87%, 88%, 89%, 90%, 91%, 92%, 93%, 94%, 95%, 96%, 97%, 98%, or 99% identity thereto, wherein at least one EAD comprises an amino acid sequence as set forth in SEQ ID NO: [3], or a sequence having at least 85%, 86%, 87%, 88%, 89%, 90%, 91%, 92%, 93%, 94%, 95%, 96%, 97%, 98%, or 99% identity thereto, and the fusion protein comprises at least one further EAD comprising an amino acid sequence as set forth in SEQ ID NO: [4] (i.e. PlySs2 CHAP): TTVNEALNNVRAQVGSGVSVGNGECYALASWYERMISPDATVGLGAGVGWVSGAIGDTISAKNIGSS YNWQANGWTVSTSGPFKAGQIVTLGATPGNPYGHVVIVEAVDGDRLTILEQNYGGKRYPVRNYYSAA SYRQQVVHYIT, or a sequence having at least 85%, 86%, 87%, 88%, 89%, 90%, 91%, 92%, 93%, 94%, 95%, 96%, 97%, 98%, or 99% identity thereto. In particular for this embodiment, reference is made to FIG. 3 and chimeric endolysins NC2, NC4, NC7, and E1.

In a particular embodiment, a fusion protein is provided wherein the CPP is located at the N-terminal position of said fusion protein.

In a specific aspect of the invention, the fusion protein provided herein comprises the peptide and domains in the order CPP-EAD-CBD from the N- to the C-terminal positions. In another particular embodiment, the fusion protein according to the invention comprises the peptide and domains in the order CPP-EAD-CBD-EAD from the N- to the C-terminal positions. In a more specific embodiment, the fusion proteins CPP-EAD-CBD or CPP-EAD-CBD-EAD, are characterised by a CBD comprising an amino acid sequence selected from SEQ ID NO: [1], SEQ ID NO: [2], or a sequence having at least 85% identity thereto, and/or at least one EAD comprising an amino acid sequence as set forth in SEQ ID NO: [3], or a sequence having at least 85% identity thereto. Optionally, the fusion protein comprises at least one further EAD comprising an amino acid sequence as set forth in SEQ ID NO: [4], or a sequence having at least 85% identity thereto.

As used herein, cell penetrating peptides (CPPs) are carriers with small peptide domains (generally equal to or less than 60, less than 50, or less than 40 amino acids (AA)) that can easily cross cell membranes. CPPs can facilitate cellular uptake of various molecular cargo, ranging from nanosize particles to small chemical molecules. Cell penetrating sequences can be used as extensions to (poly) peptide sequences thereby making them more permeable to cell membranes, or CPPs can be attached to other cargo molecules to enhance their cellular uptake.

In one embodiment, the N-terminal domain of the fusion protein of the invention is a functional polypeptide, in particular a CPP, wherein the function comprises the ability to lyse the cell wall of Streptococci or Staphylococci inside (bovine) mammary epithelial cells. In particular, the present invention provides a fusion protein wherein the CPP is positively charged. In a particular embodiment, the CPP of the chimeric endolysin according to the invention is equal to or less than 60, more specific equal to or less than 50, even more specific equal to or less than 40 AAs. The smaller the CPP is at the N-terminal position of the fusion protein, the more steric hindrance on the hydrolysing activity of the engineered endolysin is minimized. Furthermore, its positive charge may add to the affinity of the fusion protein for the negatively charged bacterial cell wall.

In another embodiment, the present invention provides a fusion protein wherein the CPP is selected from the list comprising: HIV-1 TAT, poly-arg (R8), any NZ2114 and derived peptides (Chen et al. 2017; incorporated by reference), Pep-1 and Penetratin (also termed Transportin). As used herein, peptide NZ2114 is a variant of plectasin that is significantly more potent than parental peptide. NZ2114-derived peptide or variants thereof can be selected from NZ16K (H1), NZ16R (H2), NZ18K (H3), NZ18R (H4), NZ16K18K (H5), NZ16K18R (H6), NZ16R18K (H7), or NZ16R18R (H8) (Table 2). For example, the N-terminal domain may be a polypeptide comprising or consisting of the amino acid of any one of SEQ ID NOs: [35 to 47], or any variant thereof having at least 80% identity, preferably at least 85% identity, more preferably at least 90% identity, even more preferably at least 95% identity, even more preferably at least 96% identity, even more preferably at least 97% identity, even more preferably at least 98% identity, even more preferably at least 99% identity, even more preferably at least 99.5% identity, and most preferably at least 99.7% identity resp. with the amino acid sequence of any one of SEQ ID NOS: [35 to 47] (Table 2).

In a preferred embodiment, the fusion protein according to the invention comprises at least a part of the HIV-1 TAT sequence, such as at least 10%, at least 20%, at least 30%; at least 40%, at least 50%, at least 60%, at least 70%, at least 80%, at least 90% or 100% of the HIV-1 TAT sequence SEQ ID NO: [39].

In a specific embodiment, the present invention provides a fusion protein comprising a CPP, at least one EAD, preferably comprising an amino acid sequence as set forth in SEQ ID NO: [3], or variant thereof; and at least one CBD comprising an amino acid sequence as set forth in SEQ ID NO: [1], or variant thereof, wherein the CPP is selected from the list comprising: HIV-1 TAT, poly-arg (R8), NZ2114, H1, H2, H3, H4, H5, H6, H7, H8, Pep-1 or Penetratin.

In a specific embodiment, the present invention provides a fusion protein comprising a CPP, at least one EAD, preferably comprising an amino acid sequence as set forth in SEQ ID NO: [3], or variant thereof; and at least one CBD comprising an amino acid sequence as set forth in SEQ ID NO: [2], or variant thereof, wherein the CPP is selected from the list comprising: HIV-1 TAT, poly-arg (R8), H1, H2, H3, H4, H5, H6, H7, H8, Pep-1 or Penetratin.

In an embodiment, one or more EADs and/or one or more CBDs and/or one or more CPPs of the fusion protein according to the invention are coupled directly, are delineated by or include a linker sequence.

A “linker sequence” refers to an amino acid sequence that joins the two portions or domains of the fusion protein as provided herein. As used herein, a “linker” or “linker sequence” may consist of a polypeptide having a length of about and between 1 and 70 amino acids, more specifically a length of about and between 2 and 60 amino acids, even more specific of about and between 2 and 50 amino acids. In a specific embodiment, one or more EADs and/or one or more CBDs further comprises a linker sequence, in particular a C-terminal linker sequence, such as a polypeptide having a length of about and between 1 and 70 amino acids. For example, the Ply1081 CW_7 domain comprises a C-terminal linker sequence of at least 20 AAs, preferably at least 30 AAs, more preferably at least 40 AAs, most preferably about 42 AAs. In a particular embodiment, the linker sequence of the Ply1081 CW_7 domain comprises or consists of amino the acid sequence: PSGNIPKTPSDAPKSEVVNSSTEPKTEETGANGKATDTKITK (SEQ ID NO: [48]).

In another specific embodiment, the PlySs2 SH3_5 domain further comprises a C-terminal linker sequence of at least 1 AA, more in particular an AA sequence consisting of K.

In another specific embodiment, the PlySs2 CHAP domain further comprises a C-terminal linker sequence of at least 5 AAs, preferably at least 10 AAs, more preferably at least 12 AAs, most preferably about 14 AAs. In a particular embodiment, the linker sequence of the PlySs2 CHAP domain comprises or consists of the amino acid sequence: PPGTVAQSAPNLAG (SEQ ID NO: [49]).

In another specific embodiment, the PlySs9 Amidase 3 domain further comprises a C-terminal linker sequence of at least 1 AA, preferably at least 2 AAs, more preferably at least 3 AAs, most preferably about 4 AAs. In a particular embodiment, the linker sequence of the PlySs9 Amidase 3 domain comprises or consists of the amino acid sequence: KGHS (SEQ ID NO: [50]).

In a particular embodiment, the fusion protein may further comprise a “position marker” that is preferably used to effectively attach or “click” a particular EAD or CBD domain, linker or CPP sequence with another EAD or CBD domain or linker or CPP. In the context of the present invention, the term “position marker” is an amino acid sequence that covalently links the polypeptides/domains (e.g. a particular EAD or CBD domain, linker or CPP sequence) as identified herein to form a fusion protein. More specific, the position marker comprises at least one peptide bond, i.e. 1, 2, 3, 4 or more peptide bonds. As appreciated by one of skill in the art, the position marker can comprise various types of amino acids, such as acidic, basic or neutral amino acids. In a particular embodiment, the position marker comprises a polypeptide comprising or consisting of the amino acid sequence (i) (XX) n, wherein n is 1, 2, 3, 4, 5 or 6, and wherein each X can be independently G, A, K, Y or S. Specific examples of possible click sequences include: GA, AG, GS, SG, YK and KY, more particular GA, AG, GS and KY.

For example, the TAT CPP may further comprise a click sequence comprising two amino acids at the C-terminal domain (i.e. MYGRKKRRQRRRGA; SEQ ID NO: [51]).

In a very specific embodiment, the fusion protein according to the invention comprises an amino acid sequence as set forth in SEQ ID NO: [17], or a sequence having at least 85%, 86%, 87%, 88%, 89%, 90%, 91%, 92%, 93%, 94%, 95%, 96%, 97%, 98%, or 99% identity thereto. In particular for this embodiment, reference is made to FIG. 3 chimeric endolysin NC5.

In a very specific embodiment, the fusion protein according to the invention comprises an amino acid sequence as set forth in SEQ ID NO: [11], or a sequence having at least 85%, 86%, 87%, 88%, 89%, 90%, 91%, 92%, 93%, 94%, 95%, 96%, 97%, 98%, or 99% identity thereto. In particular for this embodiment, reference is made to FIG. 3 chimeric endolysin NA1.

In a very specific embodiment, the fusion protein according to the invention comprises an amino acid sequence as set forth in SEQ ID NO: [12], or a sequence having at least 85%, 86%, 87%, 88%, 89%, 90%, 91%, 92%, 93%, 94%, 95%, 96%, 97%, 98%, or 99% identity thereto. In particular for this embodiment, reference is made to FIG. 3 chimeric endolysin NA2.

In a very specific embodiment, the fusion protein according to the invention comprises an amino acid sequence as set forth in SEQ ID NO: [13], or a sequence having at least 85%, 86%, 87%, 88%, 89%, 90%, 91%, 92%, 93%, 94%, 95%, 96%, 97%, 98%, or 99% identity thereto. In particular for this embodiment, reference is made to FIG. 3 chimeric endolysin NC1.

In a very specific embodiment, the fusion protein according to the invention comprises an amino acid sequence as set forth in SEQ ID NO: [14], or a sequence having at least 85%, 86%, 87%, 88%, 89%, 90%, 91%, 92%, 93%, 94%, 95%, 96%, 97%, 98%, or 99% identity thereto. In particular for this embodiment, reference is made to FIG. 3 chimeric endolysin NC2.

In a very specific embodiment, the fusion protein according to the invention comprises an amino acid sequence as set forth in SEQ ID NO: [15], or a sequence having at least 85%, 86%, 87%, 88%, 89%, 90%, 91%, 92%, 93%, 94%, 95%, 96%, 97%, 98%, or 99% identity thereto. In particular for this embodiment, reference is made to FIG. 3 chimeric endolysin NC3.

In a very specific embodiment, the fusion protein according to the invention comprises an amino acid sequence as set forth in SEQ ID NO: [16], or a sequence having at least 85%, 86%, 87%, 88%, 89%, 90%, 91%, 92%, 93%, 94%, 95%, 96%, 97%, 98%, or 99% identity thereto. In particular for this embodiment, reference is made to FIG. 3 chimeric endolysin NC4.

In a very specific embodiment, the fusion protein according to the invention comprises an amino acid sequence as set forth in SEQ ID NO: [18], or a sequence having at least 85%, 86%, 87%, 88%, 89%, 90%, 91%, 92%, 93%, 94%, 95%, 96%, 97%, 98%, or 99% identity thereto. In particular for this embodiment, reference is made to FIG. 3 chimeric endolysin NC6.

In a very specific embodiment, the fusion protein according to the invention comprises an amino acid sequence as set forth in SEQ ID NO: [19], or a sequence having at least 85%, 86%, 87%, 88%, 89%, 90%, 91%, 92%, 93%, 94%, 95%, 96%, 97%, 98%, or 99% identity thereto. In particular for this embodiment, reference is made to FIG. 3 chimeric endolysin NC7.

In a very specific embodiment, the fusion protein according to the invention comprises an amino acid sequence as set forth in SEQ ID NO: [20], or a sequence having at least 85%, 86%, 87%, 88%, 89%, 90%, 91%, 92%, 93%, 94%, 95%, 96%, 97%, 98%, or 99% identity thereto. In particular for this embodiment, reference is made to FIG. 3 chimeric endolysin NC8.

In a very specific embodiment, the fusion protein according to the invention comprises an amino acid sequence as set forth in SEQ ID NO: [21], or a sequence having at least 85%, 86%, 87%, 88%, 89%, 90%, 91%, 92%, 93%, 94%, 95%, 96%, 97%, 98%, or 99% identity thereto. In particular for this embodiment, reference is made to FIG. 3 chimeric endolysin NC9.

In a very specific embodiment, the fusion protein according to the invention comprises an amino acid sequence as set forth in SEQ ID NO: [22], or a sequence having at least 85%, 86%, 87%, 88%, 89%, 90%, 91%, 92%, 93%, 94%, 95%, 96%, 97%, 98%, or 99% identity thereto. In particular for this embodiment, reference is made to FIG. 3 chimeric endolysin E1.

Chimeric endolysin NC5 (see FIG. 3) demonstrated improved lytic activity compared to NC3 and NC8, as the HIV-1 TAT peptide fused to NC5 has a smaller size and a higher pl (causing a higher positive charge of the fusion protein at the pH of milk (latter having a pH in the range of 6.5 to 7.5), resulting in higher affinity for the negatively charged bacterial cell wall) compared to the CPPs that are fused to NC3 and NC8 (Table 2).

In another aspect of the invention, the fusion protein may be fused to a polypeptide or protein in order to facilitate purification of said fusion protein. Examples of such fusions are well known to those skilled in the art. Similarly, the fusion protein may be fused to an oligo-hist tag such as His or to an epitope recognized by an antibody such as well-known Myc tag epitope. Fusions to any fragment variant or derivative of an endolysin according to the present invention are also included in the scope of the invention, It will be appreciated that fusions (or variants or derivatives thereof) which retain desirable properties, namely endolysin activity are preferred. It is also particularly preferred if the fusions are ones which are suitable for use in methods described herein. For example, the fusion may comprise a further portion which confers a desirable feature on the fusion protein of the invention; for example, the portion may be useful in detecting or isolating the endolysin, promoting cellular uptake of the endolysin, or directing secretion of the protein from a cell. The portion may be, for example, a biotin moiety, a radioactive moiety, a fluorescent moiety, for example a small fluorophore or a green fluorescent protein (GFP) fluorophore, as well known to those skilled in the art. The moiety may be an immunogenic tag, for example a Myc tag, as known to those skilled in the art or may be a lipophilic molecule or polypeptide domain that is capable of promoting cellular uptake of the endolysin, as known to those skilled in the art.

In a particular embodiment, the fusion protein according to the invention further comprises a C-terminal poly-his tag such as HIS which is particularly suitable for purification purposes.

In a further aspect, an isolated nucleic acid encoding the fusion protein according to the invention is provided. In this context, the nucleic acid contains variants of its conservative substitutions (e.g. substitution of degenerate codons) and complementary sequences. The terms “nucleic acid” and “polynucleotide” are synonymous and include genes, cDNA molecules, mRNA molecules, and fragments thereof such as oligonucleotides. In a further embodiment, the nucleic acid sequence can be ‘codon-optimized’ in view of the used expression system or host organism as generally known to the skilled person. The term “codon-optimized” as it refers to genes or coding regions of nucleic acid molecules for transformation of various hosts, refers to the alteration of codons in the gene or coding regions of the nucleic acid molecules to reflect the typical codon usage of the host organism without altering the polypeptide encoded by the DNA. Such optimization includes replacing at least one, or more than one, or a significant number, of codons with one or more codons that are more frequently used in the genes of that organism.

In a specific embodiment, the present invention provides isolated nucleic acids wherein said nucleic acids comprises a nucleic acid sequence as set forth in SEQ ID NO: [5, 6, 7 or 8], or a sequence having at least 65%, 70%, 75%, 80%, 85%, 90%, 95%, 96%, 97%, 98%, or 99% identity thereto encoding corresponding amino acid sequence as set forth in resp. SEQ ID NO: [1, 2, 3, 4]. In particular, nucleic acid sequences include the nucleotide sequence of any one or more of the sequences as described herein encoding the fusion protein according to the invention.

In another specific embodiment, the present invention provides an isolated nucleic acid wherein said nucleic acids comprises a nucleic acid sequence as set forth in SEQ ID NO: [5], or a sequence having at least 65%, 70%, 75%, 80%, 85%, 90%, 95%, 96%, 97%, 98%, or 99% identity thereto encoding an amino acid sequence comprising an amino acid sequence as set forth in SEQ ID NO: [1] or a sequence having at least 85%, 86%, 87%, 88%, 89%, 90%, 91%, 92%, 93%, 94%, 95%, 96%, 97%, 98%, or 99% identity thereto.

In a particular embodiment, the present invention provides an isolated nucleic acid wherein said nucleic acids comprises a nucleic acid sequence as set forth in SEQ ID NO: [10], or a sequence having at least 65%, 70%, 75%, 80%, 85%, 90%, 95%, 96%, 97%, 98%, or 99% identity thereto encoding an amino acid sequence comprising an amino acid sequence as set forth in SEQ ID NO: [9] or a sequence having at least 85%, 86%, 87%, 88%, 89%, 90%, 91%, 92%, 93%, 94%, 95%, 96%, 97%, 98%, or 99% identity thereto.

In a very specific embodiment, the present invention provides an isolated nucleic acid wherein said nucleic acids comprises a nucleic acid sequence as set forth in SEQ ID NO: [29], or a sequence having at least 65%, 70%, 75%, 80%, 85%, 90%, 95%, 96%, 97%, 98%, or 99% identity thereto encoding an amino acid sequence comprising an amino acid sequence as set forth in SEQ ID NO: [17], or a sequence having at least 85%, 86%, 87%, 88%, 89%, 90%, 91%, 92%, 93%, 94%, 95%, 96%, 97%, 98%, or 99% identity thereto.

In a very specific embodiment, the present invention provides an isolated nucleic acid wherein said nucleic acids comprises a nucleic acid sequence as set forth in SEQ ID NO: [23], or a sequence having at least 65%, 70%, 75%, 80%, 85%, 90%, 95%, 96%, 97%, 98%, or 99% identity thereto encoding an amino acid sequence comprising an amino acid sequence as set forth in SEQ ID NO: [11], or a sequence having at least 85%, 86%, 87%, 88%, 89%, 90%, 91%, 92%, 93%, 94%, 95%, 96%, 97%, 98%, or 99% identity thereto.

In a very specific embodiment, the present invention provides an isolated nucleic acid wherein said nucleic acids comprises a nucleic acid sequence as set forth in SEQ ID NO: [24], or a sequence having at least 65%, 70%, 75%, 80%, 85%, 90%, 95%, 96%, 97%, 98%, or 99% identity thereto encoding an amino acid sequence comprising an amino acid sequence as set forth in SEQ ID NO: [12], or a sequence having at least 85%, 86%, 87%, 88%, 89%, 90%, 91%, 92%, 93%, 94%, 95%, 96%, 97%, 98%, or 99% identity thereto.

In a very specific embodiment, the present invention provides an isolated nucleic acid wherein said nucleic acids comprises a nucleic acid sequence as set forth in SEQ ID NO: [25], or a sequence having at least 65%, 70%, 75%, 80%, 85%, 90%, 95%, 96%, 97%, 98%, or 99% identity thereto encoding an amino acid sequence comprising an amino acid sequence as set forth in SEQ ID NO: [13], or a sequence having at least 85%, 86%, 87%, 88%, 89%, 90%, 91%, 92%, 93%, 94%, 95%, 96%, 97%, 98%, or 99% identity thereto.

In a very specific embodiment, the present invention provides an isolated nucleic acid wherein said nucleic acids comprises a nucleic acid sequence as set forth in SEQ ID NO: [26], or a sequence having at least 65%, 70%, 75%, 80%, 85%, 90%, 95%, 96%, 97%, 98%, or 99% identity thereto encoding an amino acid sequence comprising an amino acid sequence as set forth in SEQ ID NO: [14], or a sequence having at least 85%, 86%, 87%, 88%, 89%, 90%, 91%, 92%, 93%, 94%, 95%, 96%, 97%, 98%, or 99% identity thereto.

In a very specific embodiment, the present invention provides an isolated nucleic acid wherein said nucleic acids comprises a nucleic acid sequence as set forth in SEQ ID NO: [27], or a sequence having at least 65%, 70%, 75%, 80%, 85%, 90%, 95%, 96%, 97%, 98%, or 99% identity thereto encoding an amino acid sequence comprising an amino acid sequence as set forth in SEQ ID NO: [15], or a sequence having at least 85%, 86%, 87%, 88%, 89%, 90%, 91%, 92%, 93%, 94%, 95%, 96%, 97%, 98%, or 99% identity thereto.

In a very specific embodiment, the present invention provides an isolated nucleic acid wherein said nucleic acids comprises a nucleic acid sequence as set forth in SEQ ID NO: [28], or a sequence having at least 65%, 70%, 75%, 80%, 85%, 90%, 95%, 96%, 97%, 98%, or 99% identity thereto encoding an amino acid sequence comprising an amino acid sequence as set forth in SEQ ID NO: [16], or a sequence having at least 85%, 86%, 87%, 88%, 89%, 90%, 91%, 92%, 93%, 94%, 95%, 96%, 97%, 98%, or 99% identity thereto.

In a very specific embodiment, the present invention provides an isolated nucleic acid wherein said nucleic acids comprises a nucleic acid sequence as set forth in SEQ ID NO: [30], or a sequence having at least 65%, 70%, 75%, 80%, 85%, 90%, 95%, 96%, 97%, 98%, or 99% identity thereto encoding an amino acid sequence comprising an amino acid sequence as set forth in SEQ ID NO: [18], or a sequence having at least 85%, 86%, 87%, 88%, 89%, 90%, 91%, 92%, 93%, 94%, 95%, 96%, 97%, 98%, or 99% identity thereto.

In a very specific embodiment, the present invention provides an isolated nucleic acid wherein said nucleic acids comprises a nucleic acid sequence as set forth in SEQ ID NO: [31], or a sequence having at least 65%, 70%, 75%, 80%, 85%, 90%, 95%, 96%, 97%, 98%, or 99% identity thereto encoding an amino acid sequence comprising an amino acid sequence as set forth in SEQ ID NO: [19], or a sequence having at least 85%, 86%, 87%, 88%, 89%, 90%, 91%, 92%, 93%, 94%, 95%, 96%, 97%, 98%, or 99% identity thereto.

In a very specific embodiment, the present invention provides an isolated nucleic acid wherein said nucleic acids comprises a nucleic acid sequence as set forth in SEQ ID NO: [32], or a sequence having at least 65%, 70%, 75%, 80%, 85%, 90%, 95%, 96%, 97%, 98%, or 99% identity thereto encoding an amino acid sequence comprising an amino acid sequence as set forth in SEQ ID NO: [20], or a sequence having at least 85%, 86%, 87%, 88%, 89%, 90%, 91%, 92%, 93%, 94%, 95%, 96%, 97%, 98%, or 99% identity thereto.

In a very specific embodiment, the present invention provides an isolated nucleic acid wherein said nucleic acids comprises a nucleic acid sequence as set forth in SEQ ID NO: [33], or a sequence having at least 65%, 70%, 75%, 80%, 85%, 90%, 95%, 96%, 97%, 98%, or 99% identity thereto encoding an amino acid sequence comprising an amino acid sequence as set forth in SEQ ID NO: [21] or a sequence having at least 85%, 86%, 87%, 88%, 89%, 90%, 91%, 92%, 93%, 94%, 95%, 96%, 97%, 98%, or 99% identity thereto.

In a very specific embodiment, the present invention provides an isolated nucleic acid wherein said nucleic acids comprises a nucleic acid sequence as set forth in SEQ ID NO: [34], or a sequence having at least 65%, 70%, 75%, 80%, 85%, 90%, 95%, 96%, 97%, 98%, or 99% identity thereto encoding an amino acid sequence comprising an amino acid sequence as set forth in SEQ ID NO: [22] or a sequence having at least 85%, 86%, 87%, 88%, 89%, 90%, 91%, 92%, 93%, 94%, 95%, 96%, 97%, 98%, or 99% identity thereto.

The present invention also provides a vector including one or more of the above-mentioned nucleic acids.

### Production

Methods for the production of the fusion proteins (chimeric endolysins) of the present invention are well known in the art. Conveniently, the fusion protein is or comprises a recombinant protein. In one embodiment, the endolysins according to the invention are produced by standard techniques of genetic engineering comprising the use of a recombinant vector comprising a polynucleotide encoding an chimeric endolysin as described herein. Numerous expression systems can be used including bacterial plasmids and derived vectors, transposons, yeast episomes, insertion elements, yeast chromosome elements, viruses such as baculovirus, papilloma viruses such as SV40, vaccinia viruses, adenoviruses, fox pox viruses, pseudorabies viruses, retroviruses, cosmid or phagemid derivatives. The nucleotide sequence can be inserted in the recombinant expression vector by methods well known to a person skilled in the art such as, for example, those that are described in Molecular Cloning: A laboratory Manual, Sambrook et al, 4th Ed., Cold Spring Harbor Laboratory Press, Cold Spring Harbor, N. Y., 2001. The recombinant vector can include nucleic acid sequences that control the regulation, the expression, the transcription, and/or the translation of the polynucleotide encoding the chimeric endolysin, these sequences being selected according to the host cells that are used. The recombinant vector can further include nucleic acid sequences such as those encoding His tags for facilitating the purification step. Subsequently, such a recombinant vector is introduced in a host cell according to methods that are well known to a person skilled in the art, such as those described in Basic methods in molecular biology, Davis et al, 2nd ed., McGraw-Hill Professional Publishing, 1995, and Molecular Cloning: A laboratory Manual, supra, such as transfection by calcium phosphate, transfection by DEAE dextran, transfection, microinjection, transfection by cationic lipids, electroporation, transduction or infection. Hence in a third aspect, the present invention provides a vector comprising the nucleic acid as described herein.

In a further embodiment, the present invention provides a host cell expressing the fusion protein according to the invention, or a host cell comprising the nucleic acid or the vector according to the invention.

In particular, the nucleic acid sequence provided herein is operably linked to at least one regulatory sequence. “Operably linked” means that the coding sequence is linked to the regulatory sequence in a manner that allows expression of the coding sequence. Regulatory sequences are selected to direct the expression of the protein of interest in a suitable host cell, and include promoters, enhancers, and other expression control elements well known to the skilled person. In a particular embodiment, the nucleic acid of the present invention is in operable linkage to a promoter that drives expression in a host cell.

Hence, in one embodiment, the vector includes a promoter for driving expression of the nucleic acid disclosed herein, optionally a nucleic acid sequence encoding a signal peptide (also referred to as leader sequence) that secretes or integrates the peptide expression product on the membrane, the nucleic acid of the present invention, and optional a nucleic acid sequence encoding a terminator. When the expression vector is manipulated in a production strain or cell line, the vector may or may not be integrated into the genome of the host cell when introduced into the host cell. The vector usually carries a replication site and a marker sequence that can provide phenotypic selection in the transformed cell.

The host cell can be, for example, bacterial cells such as Escherichia coli, cells of fungi such as yeast cells and cells of Aspergillus, Streptomyces, insect cells, Chinese Hamster Ovary cells (CHO), C127 mouse cell line, BHK cell line of Syrian hamster cells, Human Embryonic Kidney 293 (HEK 293) cells. In a particular embodiment, the host cell is E. coli. Said host cells are then cultivated in appropriate conditions so as to produce the chimeric endolysin described herein, which can then further be purified from the culture medium or from the host cell lysate by any standard purification methods such as e.g. Immobilized-Metal Affinity Chromatography (IMAC). Hence in a fourth aspect, the present invention provides a host cell comprising the vector as described herein.

The invention furthermore encompasses a method of making the fusion protein provided herein, said method comprising the steps of:
introducing into a host cell a nucleic acid or construct encoding the fusion protein;
culturing said host cell under conditions suitable for expression of said protein; and
recovering the protein so expressed.

### Compositions

In a further aspect of the invention the present invention provides a pharmaceutical composition or combination comprising a chimeric endolysin (i.e., fusion protein) according to the first aspect of the invention, a nucleic acid according to the second aspect of the invention, a vector according to the third aspect of the invention, or a host cell according to the fourth aspect of the invention; and a pharmaceutically acceptable excipient.

As used herein, “pharmaceutical composition” means a therapeutically effective formulation, in particular for use in the methods of the invention. A “therapeutically effective dosage”, or “effective dosage”, or “therapeutically effective”, as used herein, refers to that amount which provides a therapeutic effect for a given condition and administration regimen. This is a predetermined quantity of active material, in particular the chimeric endolysin, calculated to produce a desired therapeutic effect in association with the required additive and diluent, i.e., a carrier or administration vehicle. As provided before, it is intended to mean an amount sufficient to reduce, and preferably prevent, a clinically significant deficit in the activity, function and response of a subject. Alternatively, a therapeutically effective dosage is sufficient to cause an improvement in a clinically significant condition in a subject. As is appreciated by those skilled in the art, the amount of an active material may vary depending on its specific activity. Suitable dosage amounts may contain a predetermined quantity of active composition calculated to produce the desired therapeutic effect in association with the required diluent. In the methods and use of compositions of the invention, a therapeutically effective dosage of the chimeric endolysin is provided. A therapeutically effective dosage can be determined by the ordinary skilled medical or veterinary worker based on patient characteristics, such as age, weight, sex, condition, complications, other diseases, etc., as is well known in the art. In one embodiment of the invention, the pharmaceutical composition comprises a fusion protein according to the invention. Thus, the pharmaceutical composition may comprise an amount of the fusion protein sufficient to inhibit at least in part the growth of cells of the genus Streptococcus and/or Staphylococcus in a subject who is infected or susceptible to infection with said bacteria. Preferably, the pharmaceutical composition comprises an amount of the fusion protein sufficient to damage or kill cells of the genus Streptococcus and/or Staphylococcus in the subject. It will be appreciated by a person skilled in the art that the fusion proteins of the invention are generally administered in admixture (e.g. as part of a composition) with a suitable pharmaceutical excipient, diluent or carrier selected with regard to the intended route of administration and standard pharmaceutical practice (for example, see Remington: The Science and Practice of Pharmacy, 19th edition, 1995, Ed. Alfonso Gennaro, Mack Publishing Company, Pennsylvania, USA).

Typically, the pharmaceutical composition comprises the fusion protein as described herein and a pharmaceutically acceptable excipient being any combination of suitable ingredients, carrier, and/or diluent, optionally in combination with an adjuvant.

As used herein, the term “pharmaceutically acceptable excipient” refers to any suitable combination of “ingredient”, “diluent”, “adjuvant” and/or “carrier”, which, by themselves or in combination, is not harmful to the individual receiving the composition. By “pharmaceutically acceptable” is meant a material that is not biologically or otherwise undesirable, i.e., the material may be administered to an individual along with the fusion protein of the invention without causing any undesirable biological effects or interacting in a deleterious manner with any of the other components of the pharmaceutical composition in which it is contained. A pharmaceutically acceptable carrier is preferably a carrier that is relatively non-toxic and safe to a patient at concentrations consistent with effective activity of the active ingredient so that any side effects ascribable to the carrier do not impair the beneficial effects of the active ingredient.

In an alternative embodiment of the invention, the pharmaceutical compositions do not comprise the fusion protein itself but instead comprises a nucleic acid molecule capable of expressing said fusion protein. Suitable nucleic acids, expression vectors, and host cells are as described in detail herein. For example, a recombinant probiotic may be used (LAB strain, e.g., Lactococcus lactis or a Lactobacillus sp.). In a further embodiment of the invention, the pharmaceutical compositions comprise a bacteriophage capable of expressing a fusion protein according to the invention. Thus, for treatment or prevention of bacterial infections described herein, the fusion protein of the invention may be administered as a protein, as a nucleic acid construct, vector or host cell which expresses the fusion protein, as part of a living organism which expresses the fusion protein (including bacteriophages), or by any other convenient method known in the art so as to achieve contact of the fusion protein with its bacterial target.

The composition of the invention can contain one or more fusion proteins of the invention.

In a particular embodiment, the composition of the present invention may comprise any combination of different fusion proteins as shown in FIG. 3, being NA1, NA2, NC1 to NC9 and E1, such as a combination of 1, 2, 3, 4, 5, 6, 7, 8, 9, 10, 11 or 12 of these fusion proteins. In a particular embodiment, the composition of the present invention may comprise one or more fusion proteins selected from the group comprising NC1, NC2, NC3, NC4, NC5, NC6, NC7, NC8 or NC9. In a further embodiment, the composition of the present invention may comprise one or more fusion proteins selected from the group comprising NC1, NC3, NC5, NC6 or NC8. In a very specific embodiment, the composition of the present invention comprises at least fusion protein NC5. Examples of possible combinations are compositions comprising NC1 and NC3, NC1 and NC5, NC1 and NC6, NC1 and NC8, NC3 and NC5, NC3 and NC6, NC6 and NC8, NC5 and NC6, NC5 and NC8, or NC6 and NC8.

In a specific embodiment, the composition of the present invention may comprise fusion protein NC5 in combination with NC8; alternatively NC5 in combination with NC3; alternatively NC5 in combination with NC2; alternatively NC5 in combination with NC8, NC3; alternatively NC5 in combination with NC8, NC2; alternatively NC5 in combination with NC3, NC2; alternatively NC5 in combination with NC8, NC3, NC2.

Pharmaceutical compositions of this invention may further comprise one or more pharmaceutically acceptable additional adjuvant(s) such as alum, stabilizers, antimicrobial agents, buffers, couloring agents, flavoring agents, adjuvants, and the like. The pharmaceutical composition may be in different forms such as solids, such as tablets or filled capsules, or liquids such as solutions, suspensions, aerosols, emulsions, elixirs, or capsules filled with the same, for oral use, or in the form of sterile injectable solutions for parenteral (including subcutaneous) use. Compositions of this invention may also be liquid or creamy formulations, e.g. suitable for topical or local use, including, but not limited to, aqueous or oily suspensions, solutions, creams, foams, gels, lotions, emulsions, syrups, and elixirs. The compositions may also be formulated as a dry product for reconstitution with water or other suitable vehicle before use. Such liquid preparations may contain additives including, but not limited to, suspending agents, emulsifying agents, non-aqueous vehicles and preservatives. Suspending agents include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid. Further materials as well as processing techniques and the like are set out in Part 5 of Remington's “The Science and Practice of Pharmacy”, 22nd Edition, 2012, University of the Sciences in Philadelphia, Lippincott Williams & Wilkins.

In a particular embodiment, the fusion protein or composition of the invention is administered locally, more specific locally to the mammary gland, even more particular intraductally such as through (one of) the teat canal(s) of the mammary gland/udder, or on the teat apex such as through dipping. Injectable compositions are typically based upon injectable PBS or other injectable carriers known in the art. The composition of this invention may also be formulated as topical or transdermal formulations comprising aqueous or non-aqueous vehicles including, but not limited to, creams, ointments, lotions, pastes, medicated plaster, patch, or membrane. In a further embodiment, the fusion protein or composition may be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

### Combinations

In a further embodiment, the fusion protein of the present invention can be administered alone or in combination with a co-agent useful in the prevention and/or treatment of Streptococcus infections or disorders such as inflammation of the mammary gland, in particular mastitis, including those caused by Streptococcus uberis, Streptococcus dysgalactiae and/or Streptococcus agalactiae, and optionally other Gram-positive bacteria such as Staphylococci like Staphylococcus aureus.

More specific, the fusion protein or composition according to the invention can be administered in combination with one or more conventional antibiotic treatments, in particular a beta-lactam antibiotic, more in particular penicillins. Examples of penicillin antibiotics that are particularly suitable (e.g. for the treatment of mammary gland infections or (bovine) mastitis) are cloxacillin, dicloxacillin, flucloxacillin, methicillin, nafcillin, or oxacillin.

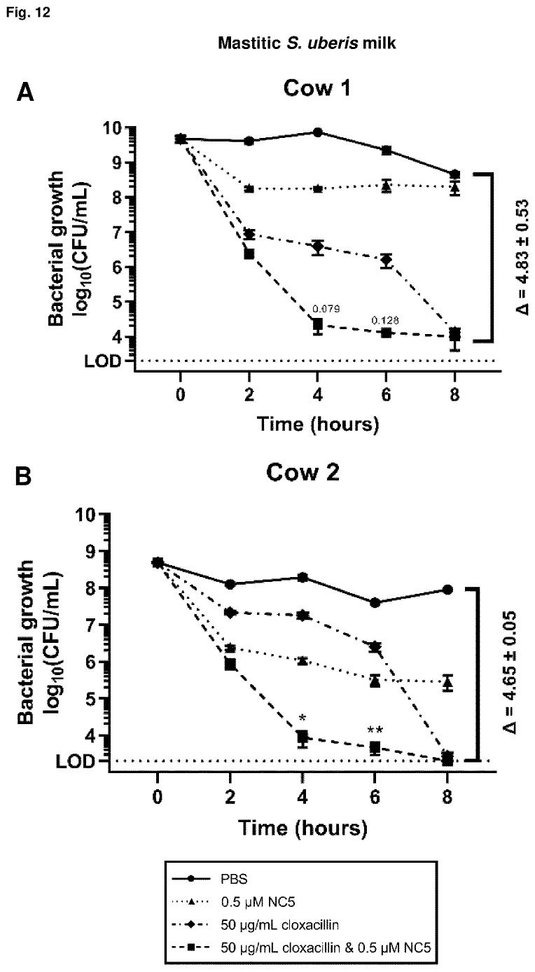

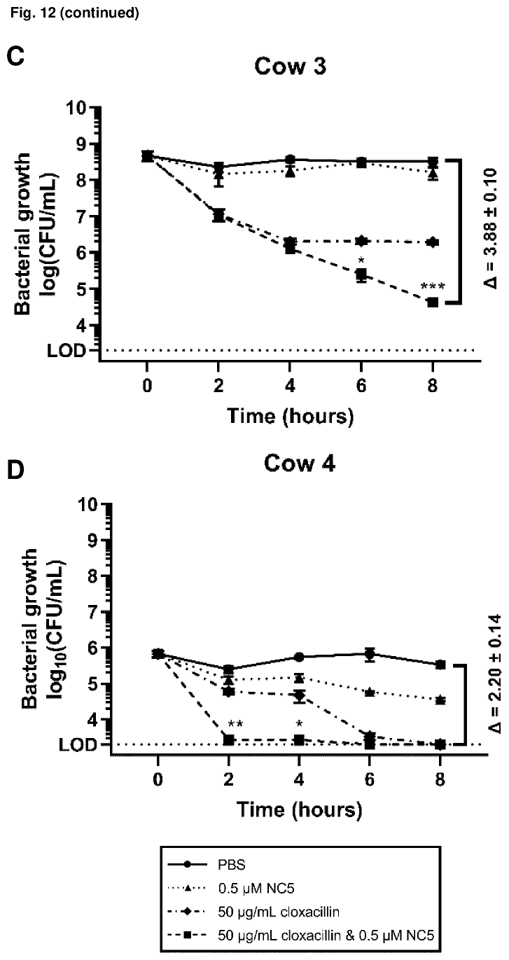

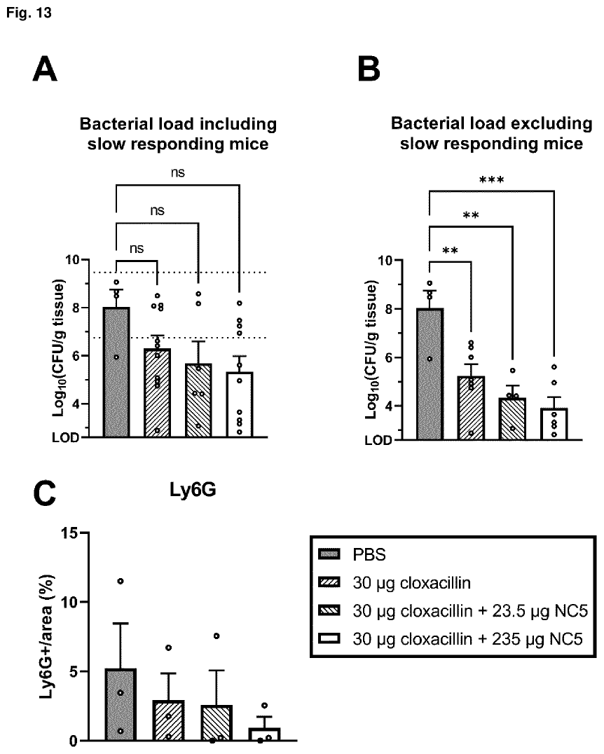

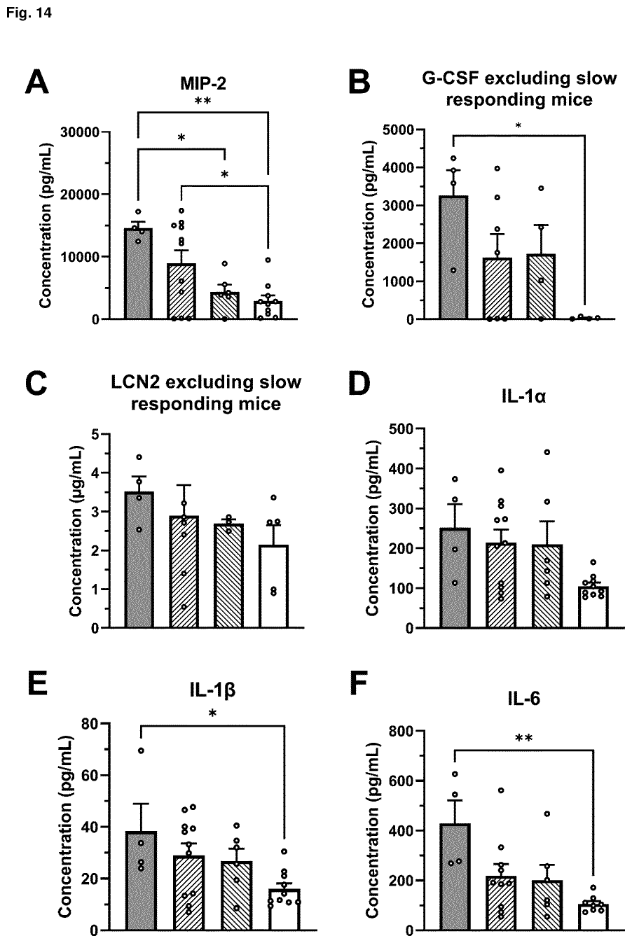

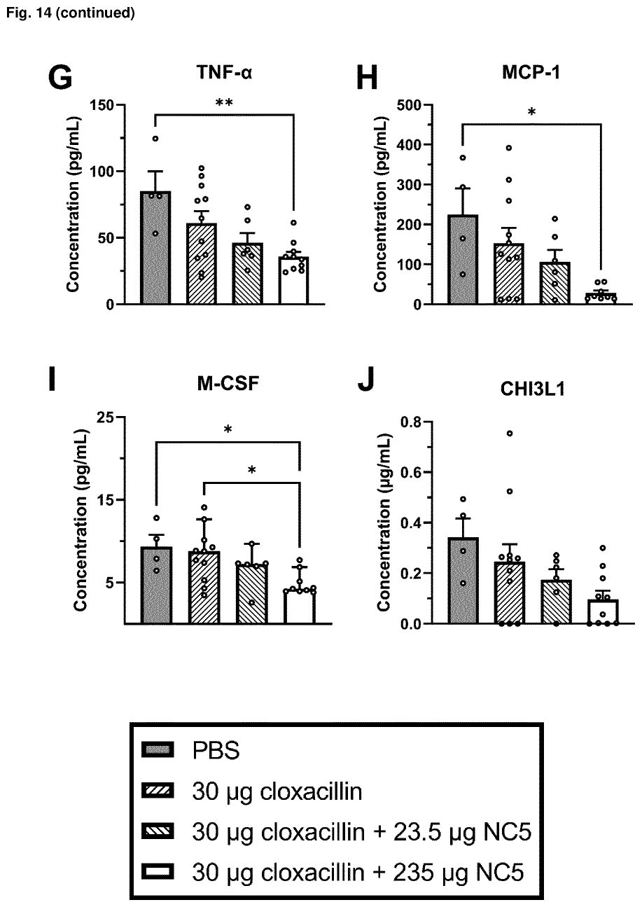

In a particular embodiment, the pharmaceutical composition according to the invention, further comprises at least one antibiotic, in particular a beta-lactam antibiotic, more in particular penicillin, even more in particular cloxacillin. As shown in the examples (FIG. 12, FIG. 13, FIG. 14), it was specifically observed that a combined administration of the fusion protein according to the invention and an antibiotic caused a significant reduction of bacterial growth. This reduction was attained on average 4 h faster than antibiotic treatment alone. In a particular embodiment, the pharmaceutical composition according to the invention which further comprises at least one antibiotic such as cloxacillin is particularly suitable in reducing bacterial growth in subjects who respond fast after the administration, in particular in subjects who respond within 4 h after administration. In a preferred embodiment, the pharmaceutical composition comprises at least 5 μg cloxacillin, such as at least 10, 15, 20, 25, 30, 35, 40, 45, 50 μg cloxacillin in combination with at least 5 μg dose of endolysin NC5 such as at least 10, 15, 20, 50, 75, 100, 125, 150, 175, 200, 225 μg dose of endolysin NC5. In another preferred embodiment, the pharmaceutical composition comprises 15-50 μg cloxacillin in combination with 23.5-235 μg dose of endolysin NC5.

Furthermore, the fusion protein or composition according to the invention can be administered in combination with one or more additional natural or chimeric endolysins, or nucleic acid molecules, vectors, host cell or bacteriophage capable of expressing the same.

In one embodiment, the combination is provided as a kit or parts, optionally including instructions for use.

### Uses

As mentioned before, the fusion protein of the present invention can be administered alone, as a pharmaceutical composition, or as a pharmaceutical composition in combination with an antibiotic (i.e., add-on therapy).

In a further embodiment, the present invention provides a fusion protein according to the invention, a nucleic acid according to the invention, a vector according to the invention, a host cell according to the invention, a (pharmacological) composition according to the invention, or kit according to the invention for use in medicine, such as veterinary medicine.

In a particular embodiment, the present invention provides a fusion protein, composition or combination for use in killing and/or reducing or preventing the growth or biofilm formation of bacteria in vivo or ex vivo, even more in particular for preventing, treating or reducing bacterial infections of the mammary gland, wherein the bacteria are from the genus Streptococcus or Staphylococcus, more specific selected from the group consisting of Streptococcus uberis, Streptococcus dysgalactiae, and Streptococcus agalactiae or Staphylococcus aureus. As used herein, an “infection” refers to a bacterial infection, more specific an infection with gram-positive bacteria, even more specific a bacterial infection caused by bacteria from the genus Streptococcus or Staphylococcus, such as Streptococcus uberis, Streptococcus dysgalactiae, Streptococcus agalactiae and/or Staphylococcus aureus; in particular Streptococcus uberis, Streptococcus dysgalactiae and/or Streptococcus agalactiae. In another embodiment, the bacterial infection results in biofilm-formation and/or an inflammation of (part of) the mammary gland, such as the breast or udder. In particular, the invention provides the fusion protein, the nucleic acid, the vector, the host cell, the (pharmacological) composition, or the combination as provided herein for preventing, treating and/or reducing mastitis or symptoms of mastitis, in particular clinical and/or subclinical mastitis. Mastitis is an infectious disease which causes severe inflammation of the mammary gland and udder tissue of dairy cattle/ruminants. It usually occurs as an immune response to bacterial invasion of the teat canal. Mastitis can occur as easily recognizable clinical mastitis, or mastitis can exist within the herd in its subclinical form, with few, if any, symptoms present in the latter case.

In yet another embodiment, the present invention provides a fusion protein, composition or combination for use in the treatment of an infection, wherein the infection is of (part of) the mammary gland, in particular of the udder more in particular of the bovine/ruminant udder.

In a specific embodiment, the present invention provides a fusion protein, composition or combination for use in the treatment, wherein the subject is a mammal, in particular a cow, buffalo, goat, sheep, camel, yak, horse, reindeer or donkey, in particular a (dairy) cow.

In a very specific embodiment, the present invention provides a fusion protein or composition for use in (dairy) cow/ruminants.

In a further aspect, the present invention provides a method of treating an infection or disease in a subject comprising administering to said subject an effective dosage of a fusion protein, a nucleic acid, a vector, a host cell, a pharmaceutical composition or combination according the invention.

In a specific embodiment, the present invention provides a method of treating an infection or disease wherein the subject has mastitis and wherein said administration is effective for reducing the severity of said mastitis, reducing or preventing bacterial biofilm formation and/or reducing the bacterial growth in the milk retrieved from the subject.

In particular, the invention provides the use of a fusion protein, a nucleic acid, a vector, a host cell, or pharmacological composition of the present invention, in the manufacture of a medicament for treating bacterial infections and disorders as provided herein.

A further aspect of the invention provides a method for killing and/or inhibiting/preventing the growth of bacteria, in particular Streptococcus uberis, Streptococcus dysgalactiae, Streptococcus agalactiae and/or Staphylococcus aureus in vivo or ex vivo, in a subject or a sample (e.g. milk) or the environment, said method comprising administering or applying the fusion protein, nucleic acid, vector, host cell, or composition of the invention. In a specific embodiment, the use as provided herein results in reducing the severity of inflammation of the mammary gland, reducing or treating mastitis, reducing or preventing bacterial biofilm formation and/or reducing the bacterial growth in the milk retrieved from the subject. For example, the fusion proteins having said activity may be used to clean surfaces which may be susceptible to contamination with such bacterial cells.

In another embodiment, the fusion protein, nucleic acid, vector, host cell, (pharmaceutical) composition according to the invention, is administered in a single dose. Alternatively, administration of a plurality of doses is also envisaged (for example, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more doses). Administration is preferably at a frequency sufficient to maintain a continuous presence of the fusion protein at the mammary gland of the subject. Preferably, the dose and dosage frequency is sufficient to prevent occurrence or recurrence of a disease or condition associated with Streptococcus and/or Staphylococcus in a subject. In particular, the dose and dosage frequency is sufficient to prevent occurrence or recurrence of bacterial growth in a subject.

It will be appreciated that the compositions described herein may be administered to a subject in combination with one or more additional therapeutic agents. For example, the fusion proteins described herein may be administered to a subject in combination with one or more conventional antibiotic treatments, one or more additional chimeric or natural endolysin, or any other therapeutic agent.

### EXAMPLES

### Example 1—Screening of Designer Endolysin Constructs

### 1.1. Construction and Screening of 6 Designs Consisting of 88,704 Theoretical Combinations

### 1.1.1. Material and Methods

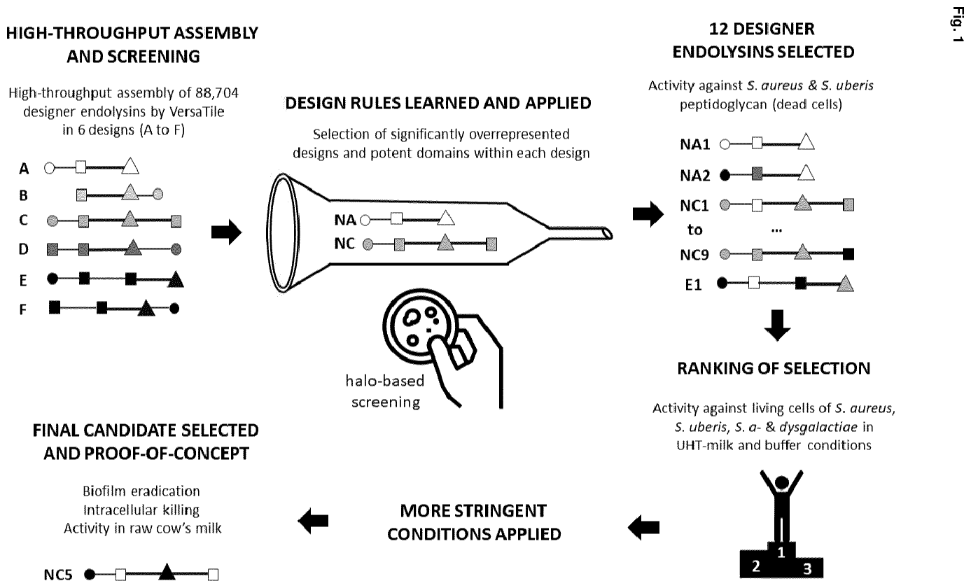

FIG. 1 displays a general overview of the high throughput assembly, screening and hit-to-lead selection of 6 libraries containing 88,704 designer endolysins.

### Bio-Informatic Analysis

S. uberis or S. suis genomes were derived from NCBI GenBank and screened for intact prophages. Subsequently, genes in the discovered prophage genomes were annotated to identify endolysins. Multiple sequence alignments were consistently performed by Clustal Omega using the MAFFT algorithm in combination with a Pearson/FASTA output. The percentages given were always derived from the percentage identity matrix that was calculated. Endolysin subdomains were delineated by combining InterPro and Phyre2 analyses.

### Cloning of (Endolysin) Genes and VersaTile Assembly of Chimers

Wildtype endolysins were codon optimized for expression in E. coli and synthesized (Twist Bioscience, USA) with a 5′ and 3′ BsaI restriction site. Subsequently, these synthesized gene fragments were introduced into pVTD3 (as destination vector) by Type IIs cloning [Gerstmans et al. 2020]]. The endolysin subdomains were thereafter subcloned starting from the full-length endolysins in the entry vector (pVTEIII), yielding tiles. Briefly, tiles were made in the entry vector pVTEIII by VersaTile cloning with the restriction enzyme SapI. Subsequently, chimers were assembled with BsaI in a cyclic restriction ligation reaction. A 4-way system was chosen with pVTD1, pVTD2 or pVTD3 as destination vectors depending on the number and order of tiles to be assembled and the desired position of the Hiss tag. All these destination vectors contain a T7 promotor.

### Bacterial Strains and Culture Conditions

The Gram-positive bovine mastitis isolates S. uberis 0140J and S. aureus Newbould 305 (N305), respectively known as S. uberis ATCC BAA-854 and S. aureus ATCC 29740, were used as reference strains. All other S. uberis and S. aureus strains included are recent (i.e., <5 years) strains isolated by the Milk Control Center of Flanders (MCC Vlaanderen) from (sub) clinical bovine mastitis cases originating from various dairy farms located in Flanders (Belgium). These isolates were complementary verified by MALDI-TOF MS. Staphylococci and streptococci were grown at 37° C. in tryptic soy broth (TSB) or brain heart infusion (BHI) (Oxoid, Belgium), respectively. Plating was conducted on tryptic soy agar (TSA) (Oxoid, Belgium) for staphylococci or brain heart infusion (Oxoid, Belgium) with the addition of 15.0 g/L agar (BHI-agar) (Chem Lab, Belgium) for streptococci. E. coli harboring the cloned constructs were grown in Lysogeny Broth (LB) (for 1.0 L: 10.0 g tryptone, 10.0 g NaCl and 5.0 g yeast extract) or Terrific Broth (TB) (for 1.0 L: 12.0 g tryptone, 24.0 g yeast extract, 4.0 mL 100% glycerol, 0.72M K₂HPO₄ and 0.17M KH₂PO₄) containing either 100.0 μg/mL ampicillin or 50.0 μg/mL kanamycin (Carl Roth, Germany). Selective plates that were used consisted of the same recipe, but 15.0 g/L agar (Chem Lab, Belgium) was added.

### 1.1.2. Results

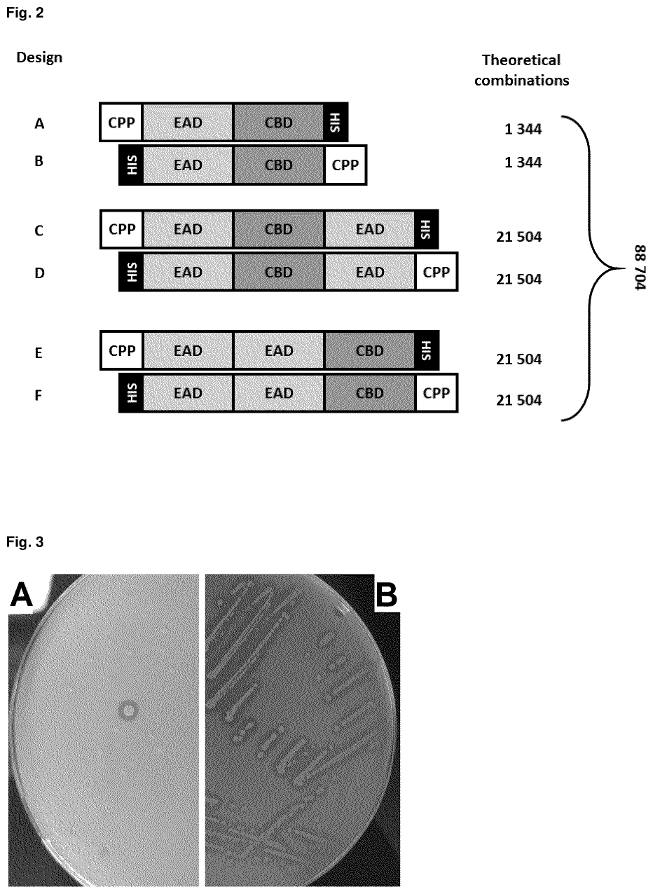

A selection of bi-specific endolysins with known activity against S. uberis and S. aureus was made. This included the EADs and CBDs from the natural endolysins PlySs2, PlyC, LysRODI, LysC1C, LysA72, PlySs9, Ply700, PlyASA2 and PlyGRCS (Table 1). This selection was subsequently supplemented with multiple EADs and CBDs that were bio-informatically discovered in intact prophage elements present in S. suis and S. uberis genomes derived from the NCBI Genbank. A multiple sequence alignment was performed and the protein sequences of the endolysin subdomains that shared less than 85.0% and 87.5% homology for S. suis and S. uberis, respectively, were selected for tile construction. As such, the amidase_5, CW_7 and glycosaminidase subdomains of PlyHA0609, the amidase_5 and CW_7 subdomain of Ply1081, the CW_7 subdomain of PlySH0104 and SH3_4 subdomains of PlyNCTC3858 and -4674 were found eligible (Table 1). To also intracellularly target S. uberis and S. aureus, 5 CPPs (i.e., oligo-arg, TAT, Penetratin, Pep-1 and H2) were selected and integrated as tiles (Table 2). After selecting the EADs, CBDs and CPPs to create and screen for bispecific designer endolysins by VersaTile assembly, six architectures were chosen in a 4-way assembly system to construct a total of 88,704 theoretical variants (FIG. 2). The designs A & B, C & D and E & F consisted of the same backbone, but each time the position of the CPP and His tag were mirrored (FIG. 2). It was also decided to screen without a CPP being present. Taking into account the selected 16 EADs, 14 CBDs and 6 CPPs, this resulted in 1,344 (=16×14×6) theoretical combinations for both libraries A & B, whereas this number was 21,504 (=6×16×16×14) for each of the libraries C, D, E & F (FIG. 2).

TABLE 1
The EADs and CBDs from the selected endolysins together with
the bacteriophage host genus and NCBI reference of the
(pro)phage from which the endolysins were derived.

| | Bacteriophage | (Pro)phage | | Selected |
|---|---|---|---|---|
| Endolysin | host genus | NCBI Reference | Selected EAD(s) | CBD |
| PlySs2 | Streptococcus | KC348602.1 | CHAP | SH3_5 |
| PlyC | Streptococcus | AY212251.1 | CHAP | PlyCb |
| LysRODI | Staphylococcus | KP027446.1 | CHAP, Amidase_2 | SH3_5 |
| LysC1C | Staphylococcus | KP027447.1 | CHAP, Amidase_2 | SH3_5 |
| LysA72 | Staphylococcus | EU861004.1 | CHAP, Amidase_2 | SH3_5 |
| PlySs9 | Streptococcus | KC348603.1 | CHAP, Amidase_3 | None |
| Ply700 | Streptococcus | DQ198146.1 | Amidase_5 | SH3_4 |
| PlyλSA2 | Streptococcus | AE009948.1 | Amidase_5, | CW_7 |
| | | | Glycosaminidase | |
| PlyHA0609 | Streptococcus | NZ_CP024126.1 | Amidase_5, | CW_7 |
| | | | Glycosaminidase | |
| Ply1081 | Streptococcus | NZ_CP017667.1 | Amidase_5 | CW_7 |
| PlySH0104 | Streptococcus | NZ_CP025419.1 | None | CW_7 |
| PlyNCTC3858 | Streptococcus | NZ_LS483397.1 | None | SH3_4 |
| PlyNCTC4674 | Streptococcus | NZ_LS483408.1 | None | SH3_4 |
| PlyGRCS | Streptococcus | NC_023550.1 | None | SH3_5 |

TABLE 2
The selected eukaryotic CPPs together with their protein sequence and pI.

| | | | SEQ |
|---|---|---|---|
| CPP | Protein sequence | pI | ID NO |
| Oligo-arg (R8) | MRRRRRRRR | 12.85 | [35] |
| TAT | MYGRKKRRQRRR | 12.31 | [36] |
| Penetratin | MRQIKWFQNRRMKWKK | 12.31 | [37] |
| Pep-1 | MKETWWETWWTEWSQPKKKRKV | 9.82 | [38] |
| NZ2114 | MGFGCNGPWNEDDLRCHNHCKSIKGYKGGYCAKGGFVCKCY | 8.62 | [39] |
| NZ16K (H1) | MGFGCNGPWNEDDLRCKNHCKSIKGYKGGYCAKGGFVCKCY | 8.84 | [40] |
| NZ16R (H2) | MGFGCNGPWNEDDLRCRNHCKSIKGYKGGYCAKGGFVCKCY | 8.86 | [41] |
| NZ18K (H3) | MGFGCNGPWNEDDLRCHNKCKSIKGYKGGYCAKGGFVCKCY | 8.84 | [42] |
| NZ18R (H4) | MGFGCNGPWNEDDLRCHNRCKSIKGYKGGYCAKGGFVCKCY | 8.86 | [43] |
| NZ16K18K (H5) | MGFGCNGPWNEDDLRCKNKCKSIKGYKGGYCAKGGFVCKCY | 9.02 | [44] |
| NZ16K18R (H6) | MGFGCNGPWNEDDLRCKNRCKSIKGYKGGYCAKGGFVCKCY | 9.04 | [45] |
| NZ16R18K (H7) | MGFGCNGPWNEDDLRCRNKCKSIKGYKGGYCAKGGFVCKCY | 9.04 | [46] |
| NZ16R18R (H8) | MGFGCNGPWNEDDLRCRNRCKSIKGYKGGYCAKGGFVCKCY | 9.06 | [47] |

1.2. Screening of 88,704 VersaTile-Assembled Designer Endolysins Resulted in a Top 12 Hits with Lytic Activity Against S. uberis and S. aureus Peptidoglycan

### 1.2.1. Material and Methods

### Halo-Based Screening of Active Fusion Proteins

Chemocompetent E. coli TOP10, BL21 (DE3) or BL21 (DE3) pLysS (NEB, USA) were transformed with 5 ng/μL DNA via heat shock at 42° C. after a 30 min incubation on melting ice. The transformed cells were then incubated for 1 h at 37° C. in super optimal broth (SOC) (for 1.0 L: 0.5% yeast extract, 2.0% tryptone, 10.0 mM NaCl, 2.5 mM KCl, 10.0 mM MgCl₂, 10 mM MgSO₄ and 20 mM D-glucose) after a 5 min recovery on melting ice. E. coli were subsequently plated. For rationally assembled constructs (i.e., natural endolysins PlySs2 & PlySs9 as positive controls), one colony from the overnight incubated plate was inoculated and grown in LB with the addition of a selective antibiotic and again incubated overnight. For randomly assembled VersaTile constructs (e.g., NC5), transformed E. coli BL21 (DE3) pLysS were plated on LB agar that contained 50.0 μg/mL kanamycin and 5% sucrose (Carl Roth, Germany) as selection markers, 1 mM isopropyl β-D-1-thiogalactopyranoside (ThermoFisher Scientific, USA) as inducing agent for protein expression and 1.0 or 2.0% autoclaved, washed peptidoglycan of either S. aureus N305 or S. uberis 0140J, respectively. These plates were then incubated at 37° C. for 48 hours followed by incubation at room temperature for at least another 24 hours. Colonies displaying a halo against S. aureus N305 were picked and streaked out on the same agar, but containing S. uberis 0140J peptidoglycan instead. Constructs that yielded halos against the peptidoglycan of both bacterial species were regarded as double positive hits. Those hits were along with the rationally assembled constructs inoculated and grown in LB with the addition of 50.0 μg/mL kanamycin. Long-term storage was performed at −80° C. by adding 10% glycerol in a cryovial. Pure plasmid was obtained from an overnight culture using the GeneJET plasmid miniprep kit (ThermoFisher Scientific, USA) and sent to LGC Genomics (Berlin, Germany) for Sanger sequencing. The genetic code was aligned using Benchling (Biology Software, USA). All DNA manipulations, DNA engineering and DNA alignments were executed via Benchling (Biology Software, USA).

### 1.2.2. Results

Following the construction of the six libraries of variants by VersaTile shuffling, the variants were screened with a halo assay. Therefore, E. coli BL21 (pLysS) were transformed with the assembled plasmids and plated on selective agar containing kanamycin and sucrose as selection markers, IPTG as inducing agent for protein expression and autoclaved S. aureus N305 cells. E. coli colonies harboring an active and properly-expressed chimeric designer endolysin showed a clearing zone (i.e., halo) around their respective colony (FIG. 3A). Subsequently, E. coli showing this halo were picked and streaked out onto the same agar, but containing autoclaved S. uberis 0140J cells instead (FIG. 3B). If clearing zones (i.e., “halos”) were observed against the peptidoglycan of both pathogens, the designer endolysin was regarded a ‘bispecific designer endolysin’. After identifying clearing zones, E. coli were grown for long-term storage and plasmids were Sanger sequenced. In total, eighteen hits were identified (i.e., 10 hits in architecture A (=55.55%), 7 hits in architecture C(=38.90%) and 1 hit in architecture E (=5.55%)). The different distributions per architecture and the occurrences of observed tiles at their observed position were compared to an expected distribution to assess if they differed significantly or not. As such, bispecific designer endolysins or “hits” occurred significantly more in architecture A (p<0.001; Bonferroni correction for multiple comparisons with adapted (a) of 0.1, p<0.1/6 (=0.016)) and architecture C (p=0.021) (Table 3). One additional hit (p >0.016) was found in architecture E. No active bispecific endolysins were found in architecture B, D and F.

TABLE 3
The approximate number and percentage of transformed E. coli screened for each
design, together with the respective number and percentage of bispecific hits.

| | | Approximately | | | | Binomial T-test (If | |
|---|---|---|---|---|---|---|---|
| | | screened° | | Bispecific hits | | applicable) | |
| Screening | Design | Numeric | % | Numeric | % | p-value | Significance |
| 1 | A | 3900 | 290.17 | 10 | 0.26 | <0.001 | * |
| | B | 3900 | 290.17 | 0 | 0 | — | |
| | C | 5120 | 23.81 | 7 | 0.17 | 0.021 | ns |
| | D | 6780 | 31.53 | 0 | 0 | — | |
| | E | 16 220 | 75.43 | 1 | 0.01 | 0.341 | ns |

| | | | | | | |
|---|---|---|---|---|---|---|
| | F | 4250 | 19.76 | 0 | 0 | — |
| | Total | 40 170 | 45.29 | 18 | 0.04 | — |
| 2 | NA | 145 | 725.00 | 4 | 2.76 | — |
| | NC | 275 | 343.15 | 25 | 9.09 | — |
| | Total | 420 | 420.00 | 29 | 6.90 | — |

A binomial T-test (Bonferroni correction with adapted significance level (α) of 0.1, p < 0.1/6 (=0.016)) was performed in the first screening to identify libraries that statistically contained more active designer endolysins.
°means it was assumed that every possible chimeric endolysin occurred only once in the performed screening,
* indicates a significantly different distribution compared to a uniform distribution,
‘ns’ means the observed p-value was not statistically significant.

Concerning the EADs, PlySs2 CHAP and PlySs9 amidase_3 were significantly (p<0.006; Bonferroni correction for multiple comparisons with adapted (a) of 0.1, p<0.1/16 (=0.006)) overrepresented at position 2, in comparison to the CHAP domain of LysRODI that was significantly (p<0.006) overrepresented at position 4 (Table 4). To finalize this observation, the PlySs2 SH3_5 and Ply1081 CW_7 were significantly (p<0.007; Bonferroni correction for multiple comparisons with adapted (a) of 0.1, p<0.1/14 (=0.007)) overrepresented as CBDs at position 3 (Table 4).

TABLE 4
Statistical analysis of overrepresented tiles observed in position
1 for architecture A, C and E, in position 2 & 3 for architecture
A and C, and in position 4 for architecture C.

| | | | Times observed | | Binomial T-test | |
|---|---|---|---|---|---|---|
| Position | Tile | | Numeric | % | p-value | Significance |
| 1 | CPP | No CPP | 16 | 88.89 | <0.001 | * |
| 1 | CPP | R₈ (oligoarginine) | 2 | 11.11 | 0.755 | ns |
| 2 | EAD | PlySs2 CHAP | 8 | 47.10 | <0.001 | * |
| 2 | EAD | PlySs9 amidase_3 | 7 | 41.20 | <0.001 | * |
| 2 | EAD | LysRODI CHAP | 1 | 5.85 | 1.000 | ns |
| 2 | EAD | LysRODI amidase_2 | 1 | 5.85 | 1.000 | ns |
| 3 | CBD | PlySs2 SH3_5 | 10 | 58.80 | <0.001 | * |
| 3 | CBD | Ply1081 CW_7 | 5 | 29.40 | 0.005 | * |
| 3 | CBD | PlySH0104 CW_7 | 1 | 5.90 | 1.000 | ns |
| 3 | CBD | PlyλSA2 CW_7 | 1 | 5.90 | 1.000 | ns |
| 4 | EAD | LysRODI CHAP | 4 | 57.10 | <0.001 | * |
| 4 | EAD | LysRODI amidase_2 | 1 | 14.30 | 0.364 | ns |
| 4 | EAD | PlySs9 amidase_3 | 1 | 14.30 | 0.364 | ns |
| 4 | EAD | PlyC CHAP | 1 | 14.30 | 0.364 | ns |

A binomial T-test (Bonferroni correction with adapted significance level (α) of 0.1, p < 0.1/6 (=0.016) for every CPP, p < 0.1/16 (=0.006) for every EAD, p < 0.1/14 (=0.007) for every CBD) was performed to identify the tiles that significantly were overrepresented at their respective position.
* indicates a significantly different distribution compared to a uniform distribution,
‘ns’ means the observed p-value was not statistically significant.

To proceed, it was decided to narrow-down the designs and building blocks according to the observations made and the goals set. As such, a second screening was performed by only including the significantly overrepresented designs A and C, which we hereafter refer to as narrow-down architecture A (NA) and C (NC). Only the significantly overrepresented tiles in position 2 and 3 were included, meaning only PlySs2 CHAP and PlySs9 amidase_3 were included as candidate building blocks in position 2 and PlySs2 SH3_5 and Ply1081 CW_7 in position 3. It was decided to include all CPPs in order to create endolysins with cell penetrating capacities. In position 4 (only for NC), all building blocks were included due to the limited number of hits found in the first screening (i.e., only 7).

By narrowing down the designs to NA and NC with the selected 5 CPPs in position 1, 2 EADs in position 2, 2 CBDs in position 3 and 4 EADs in position 4, a library with 100 (=(5×2×2)+(5×2×2×4)=100) theoretical combinations remained. Similar to the first screening, a halo-based plating method followed by Sanger sequencing was used to screen NA and NC in a saturating way for bispecific designer endolysins (Table 3). This narrow-down to NA and NC caused an increase of the percentage of hits found compared to A and C (2.76% and 9.09% versus 0.26% and 0.17%, respectively; Table 3). This corresponds to a 11- to 53-fold enrichment by implementing design rules identified in the first screening campaign of the library with 88,704 variants and shows the benefits of the combination of the combinatorial VersaTile technique in combination with a deep screening effort.

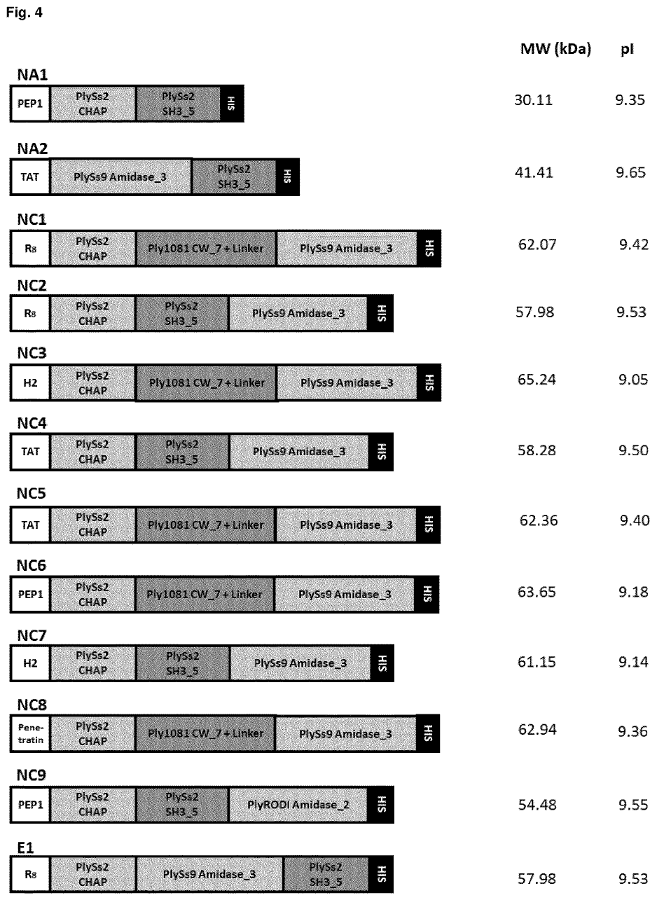

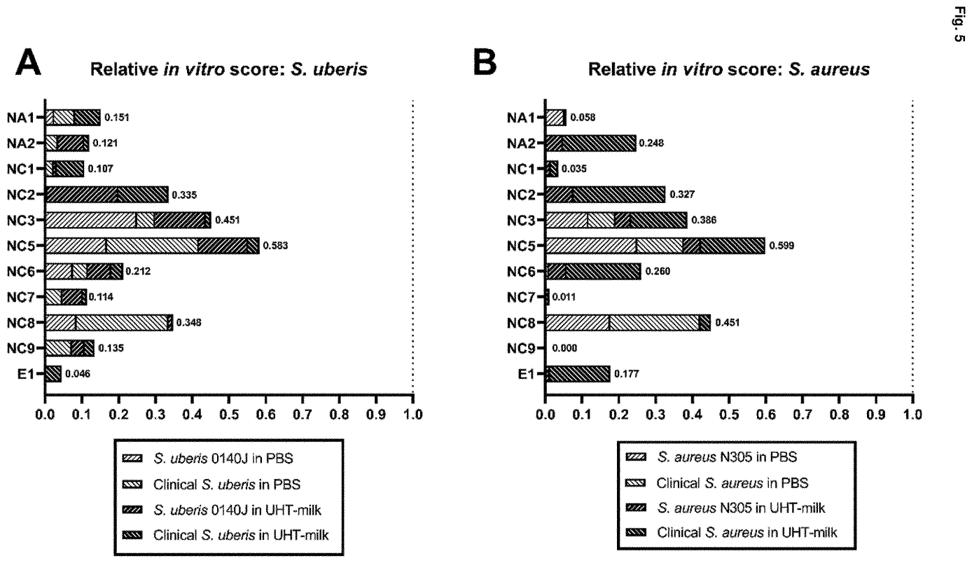

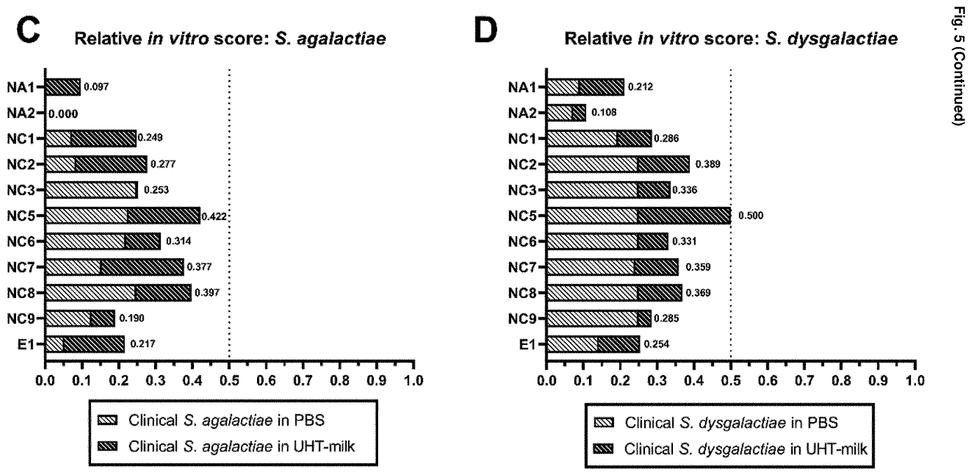

Eventually, 12 bispecific designer endolysins were discovered with lytic activity against autoclaved S. aureus N305 and S. uberis 0140J cells (FIG. 4). Within these 12 bispecific designer endolysins, 2, 9 and 1 hit belonged to architecture NA, NC and E, respectively. All hits carried the PlySs2 CHAP domains in position 2, except for hit NA2 that contained the PlySs9 amidase_3 (FIG. 4). Similarly, all hits in architecture NC carried the PlySs9 amidase_3 in position 4, except for hit NC9 that had the LysRODI amidase_2 instead. The hits in architecture NC always combined a CHAP domain with an amidase, which may result in a synergistic effect by targeting different peptidoglycan bonds. An important observation is that PlySs2 CHAP+Ply1081 CW_7+PlySs9 amidase_3 occurred in combination with all the CPPs included (i.e., NC1, NC3, NC5, NC6 & NC8), which is a strong and independent confirmation that this unexpected combination of two unrelated EADs and a third CBD from another origin stands out among other combinations. The selected CBDs in position 3 were more or less equally present in hits retrieved from architecture NC, whereas only PlySs2 SH3_5 was observed in both hits of architecture NA. Hit E1, which was found in the first screening, consisted of the same building blocks as hit NC2, but in a different order, and was retained for further analysis. All twelve hits had a pl in the range of 9.05 to 9.65, which assures a positive charge in both normal and mastitic milk (latter having a pH in the range of 6.5 to 7.5) causing the fusion protein to stay in its soluble form and possess affinity for the negatively charged bacterial cell wall. The MW of the twelve hits ranged from 30.11 to 65.24 kDa.

1.3. Challenge of the Identified Twelve Chimeric Endolysin Top Candidates with Living, Stationary Phase S. uberis and S. aureus in PBS and UHT-Milk

It is important to note that differences in enzymatic and antibacterial activity may be observed between different buffer systems such as PBS, UHT milk, raw bovine milk or mastitic milk. Therefore, and in order to create standardized conditions, the experiments described above were conducted in PBS or commercial heat-treated sterile milk. The overall best performing endolysins were selected based on in vitro cumulative performance in PBS and UHT-milk. The skilled artisan is aware that the optimal concentration and type of endolysin may vary according to the medium wherein the endolysin is used.

### 1.3.1. Materials and Methods

### Protein Expression and Purification for In Vitro Experiments

A 5.0 mL overnight culture of the transformed E. coli BL21 (DE3) was poured into a 2.0 L baffled Erlenmeyer flask filled with 0.5 L LB (or alternatively TB) and the addition of 50.0 μg/mL kanamycin. When the E. coli obtained an optical density measured at 600 nm (OD₆₀₀) of 0.6-0.8, protein expression was induced by addition of 1 mM IPTG (Carl Roth, Germany). After expression overnight at 16° C., E. coli were pelleted, supernatant was decanted and stored at −80° C. until further processing. The pellet was dissolved in PBS containing 10 mM imidazole (Carl Roth, Germany), 1 mM phenylmethylsulphonyl fluoride (Carl Roth, Germany) and 1 mM DNAse I (NEB, USA). The E. coli suspension was then sonicated on ice and ultra-centrifuged (20000 g, 4° C., 20 minutes). Subsequently, the supernatant was poured over a His GraviTrap™ column (Sigma-Aldrich, USA) for Ni-NTA chromatography. The column was washed with 10 mM imidazole in PBS (pH 7.4), 1 M NaCl (Carl Roth, Germany) in PBS (pH 7.4) and 20 mM 2-(N-morpholino) ethane sulfonic acid (MES) (Carl Roth, Germany) in PBS (pH 6.0). Finally, the target protein was eluted from the column with increasing concentrations of imidazole (20, 50, 100, 250 and 500 mM) in PBS, each time with the addition of 0.5 M NaCl and 10% glycerol (pH 7.4). The target protein in the eluted fractions was detected by SDS-PAGE on a 12% polyacrylamide gel (Bio-Rad Laboratories, USA), stained with Coomassie brilliant blue (Carl Roth, Germany) and decolorized in distilled water. Buffer exchange and concentrating the protein was executed by using Pierce™ Protein Concentrators PES with a MW cut-off (MWCO) of 10 kDa (ThermoFisher Scientific, USA). Finally, protein was filter-sterilized (PVDF membrane, 0.45 μm) by means of a syringe and the protein concentration was determined by Bradford.

### Evaluation of the Enzymatic and Antibacterial Activity in PBS and UHT-Pasteurized Milk

Concerning the time killing assay, an overnight bacterial culture was diluted 1:10 for S. uberis, S. agalactiae-& dysgalactiae or 1:100 for S. aureus in PBS or UHT-pasteurized whole cow's milk. A volume of 10 μL thereof was combined with an equal amount of chimeric endolysin in PBS (or only PBS as negative control) in triplicate and incubated in a covered 96-well microtiter plate at 37° C. for 2 h. The dose was low (i.e. 0.25 μM), but this was chosen to differentiate between less and better performing variants, consequently allowing a ranking. Finally, these fractions were serially diluted 1:10 in PBS. 5 μL of each fraction was spotted on TSA or BHI-agar and incubated overnight at 37° C. The following day, colony forming units (CFU) were counted and converted to CFU/mL to calculate logarithmic reductions in comparison with the negative control. The relative scores were calculated by dividing the observed logarithmic reduction for each endolysin by the maximal reduction observed for that specific condition and strain. Next, this value was divided by the number of conditions and strains tested (i.e., 8=4 bacterial strains×2 conditions (PBS/UHT-milk)). As such, the best performing candidate in each individual assay received a score of 0.125 (=(1/1)/8). When accumulating these individual scores over the different conditions tested, the chimeric endolysin that would perform superior in each individual assay is expected to yield the maximal score of 1.0 (=8×0.125). Turbidity reduction assays were performed by combining 100 μL of overnight grown bacterial cells with an equal volume of purified enzyme at the desired concentration. Bacterial cells were washed with PBS and resuspended in a 1:1 ratio with enzyme to an OD₆₀₀ of approximately 1.0. Next, the OD₆₀₀ was measured using a Tecan infinite 200 PRO plate reader (Tecan, Switzerland) every 15 seconds at 37° C., shaking the 96-well plate between each measurement.

### 1.3.2. Results

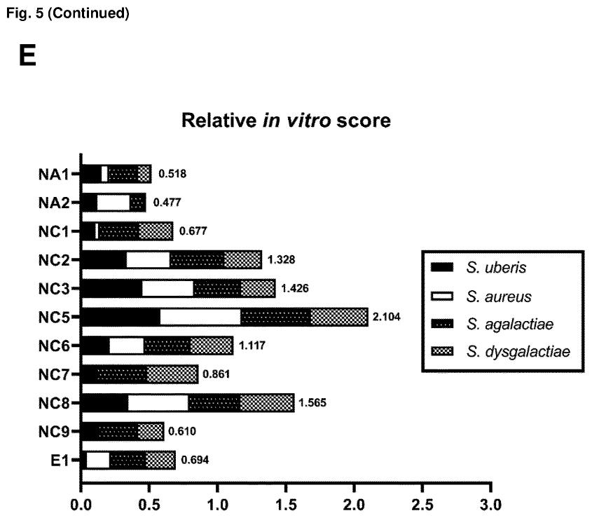

The twelve chimeric endolysins were expressed and purified (SDS-PAGE not shown). Stationary phase S. uberis 0140J or S. aureus N305 and one randomly chosen clinical isolate of S. uberis, S. aureus, S. agalactiae and S. dysgalactiae were challenged with a final concentration of 0.25 μM endolysin in a 2 h time killing assay (TKA) in both phosphate buffered saline (PBS) and ultra-high temperature pasteurized whole cow's milk (UHT-milk). NC4 could not be included in this assay as it disintegrated and precipitated during the preceding dialysis to remove the elution buffer's imidazole from the purified proteins. This can optionally be solved by using another buffer. The observed logarithmic reductions under PBS and UHT-milk conditions were subsequently converted to a relative in vitro scoring system which allowed a ranking of the leading candidates within these twelve bispecific chimeric endolysins (FIG. 5). In a next narrow-down step it was decided to continue with the four overall best performing candidates, which were identified to be NC2, NC3, NC5 and NC8.

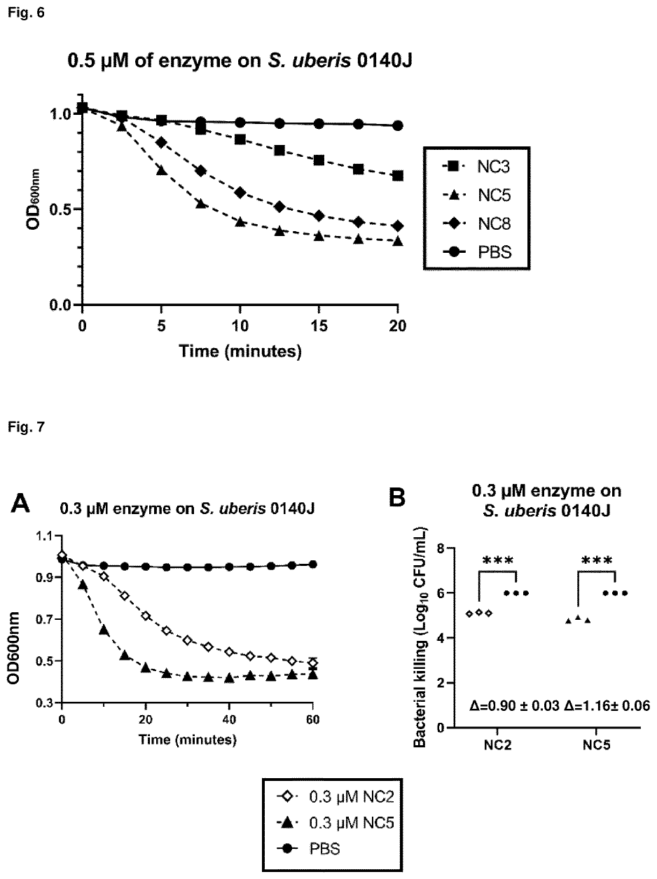

Upon this selection of NC2, NC3, NC5 and NC8, the effect of the CPP on the lytic activity of these constructs was investigated. When NC3, NC5 and NC8 were comparatively challenged at an equimolar concentration of 0.5 UM with stationary phase S. uberis 0140J in a turbidity reduction assay (TRA), it was seen that NC5 lysed the bacterial cells faster and resulted in a higher reduction of the OD₆₀₀ (FIG. 6). It can be expected that both the size and pl of the CPP influences the construct's lytic activity, as an increase of those two parameters can cause steric hindering and an improved affinity for the negatively charged bacterial cell wall, respectively. In line with this, the HIV-1 TAT peptide fused to NC5 indeed has a smaller size and a higher pl compared to the CPPs that are fused to NC3 and NC8 (Table 2).

1.4. Activity Against (Sub) Clinical Field Isolates of S. uberis, S. agalactiae-& dysgalactiae and S. aureus 1.4.1. Materials and Methods

### Whole Genome Sequencing, Sequence Typing and Determination of the Agr and Capsular (Sero) Types of Mastitis Isolates

DNA was extracted from overnight bacterial cultures according to the manufacturer's protocol using the DNeasy Ultraclean Microbial kit (Qiagen, USA). Subsequently, the genomic DNA was sequenced using Illumina MiniSeq platform at the Laboratory of Gene Technology, KU Leuven (Belgium). A library was prepared using the Nextera Flex DNA Library Kit (Illumina, USA) for each sample, tagged with a unique adapter sequence. The quality of each library preparation was controlled using an Agilent Bioanalyzer 2100. Genome assembly was performed using the Belgian Galaxy platform (SPAdes assembly algorithm: version 3.12.0). Quality of the reads was verified using FASTQC (version 1.1.5). Regarding S. uberis, multilocus sequence typing was performed. The sequencing data in FASTA format were entered into the PubMLST database (accessed on 5 Jan. 2022) to confirm the bacterial species and identify allelic matches. Each S. uberis isolate was defined by an allelic profile, which corresponds to the allele numbers at the seven loci in the order arcC, ddl, gki, recP, tdk, tpi, and yqil. According to the combination of alleles, the sequence type (ST) and global clonal complex (GCC) were determined. If an unknown ST emerged, the GCC was estimated based on multiple sequence alignment against a set of S. uberis genomes with a known GCC derived from the PubMLST database. This was also done if the allelic profile yielded a ST that was not yet assigned to a GCC in the PubMLST database. Regarding S. aureus, the agr and capsular (sero) type were determined. Multiple sequence alignment using BLASTn was performed with each S. aureus genome against the agr type I, II, III and IV genes, as well as the genes encoding capsular serotype 5 or 8.

Time killing and turbidity reduction assays were performed as previously described in 1.3.1.

### 1.4.2. Results

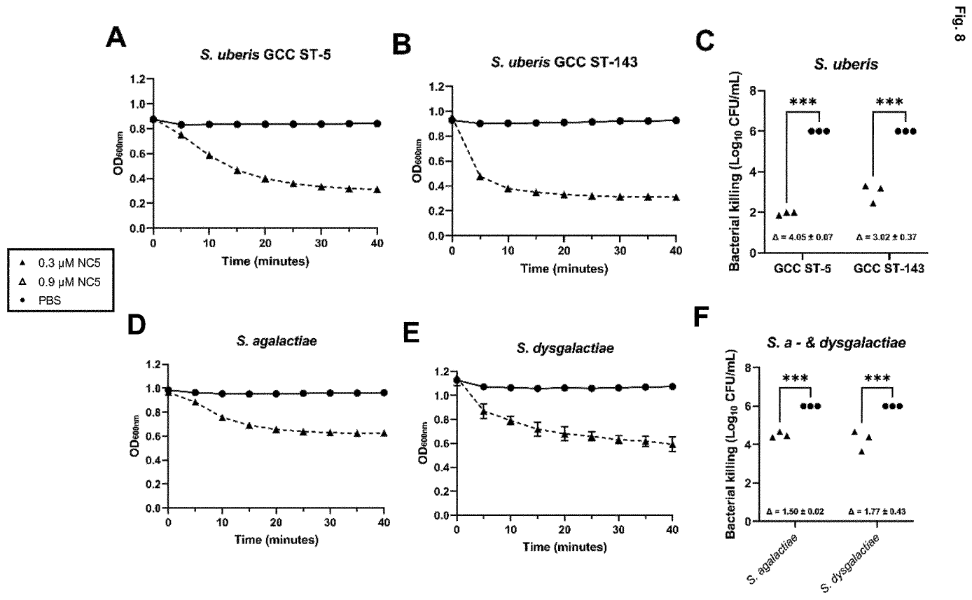

Next, the activity of NC2 and NC5 was comparatively evaluated by TRAs and time killing assays (TKAs) against S. uberis 0140J and S. aureus N305. This revealed a significant (p<0.001) reduction of the OD₆₀₀ (i.e., 0.53±0.01 ΔOD₆₀₀ for NC2; 0.57±0.00 ΔOD₆₀₀ for NC5) and log₁₀ (i.e., 0.90±0.03 Δ log₁₀ for NC2; 1.16±0.06 Δ log₁₀ for NC5) for S. uberis 0140J with 0.3 μM endolysin (FIGS. 7A&B). To validate these results, 5 clinical and 4 subclinical bovine mastitis isolates of each bacterial species were likewise challenged (Table 5). This included S. uberis isolates belonging to the global clonal complex (GCC sequence types (STs)-5 and -143, which are typically associated with (sub) clinical bovine mastitis)]. After pairwise comparing the lytic effect of NC2 versus NC5 within each S. uberis isolate challenged with 0.3 μM endolysin, it became clear that NC5 lysed the S. uberis isolates significantly (p <0.01) faster (i.e., (ΔOD600/min)/μM). At the end of the TRA and TKA, no significant (P >0.05) pairwise differences in ΔOD₆₀₀ and Δ log₁₀ against S. uberis was seen if NC2 was compared with NC5, and vice versa. This means that NC5 lysed all S. uberis strains faster, but NC2 caught up with the enzymatic activity of NC5 upon termination of the TRA. Although sensitivity to these designer endolysins was strain-dependent, enzymatic activity evaluated by a drop in OD₆₀₀ over time was demonstrated for all S. uberis strains challenged (Table 5). This enzymatic activity corresponded with bacterial killing that attained 4.05±0.07 and 3.02±0.37 log₁₀ for a GCC ST-5 and -143 S. uberis isolate, respectively (FIGS. 8 A, B&C and Table 5). Of note, a decrease of the OD₆₀₀ not always resulted in a log₁₀ reduction (e.g., clinical S. uberis 4 in Table 5). For S. aureus, one cap 5+ agr I and eight cap 8+ agr I, II or III isolates are typically associated with (sub) clinical bovine mastitis, and showed significantly (p<0.001) improved enzymatic (i.e., ΔOD₆₀₀) and killing (i.e., Δ log₁₀) activity against S. aureus for NC2 when pairwise compared to NC5. In contrast to S. uberis, there was no significant (p >0.05) difference in S. aureus lysis speed (i.e., (ΔOD600/min)/μM) between NC2 and NC5.

The lytic capability of NC5 was examined against other relevant bovine mastitis streptococci. Three clinical bovine mastitis isolates of S. agalactiae & dysgalactiae were additionally challenged with 0.3 μM NC5. This resulted in significant (p<0.001) reductions up to 0.39±0.02 ΔOD₆₀₀ and 1.50±0.02 Δ log₁₀ for the S. agalactiae isolates, and 0.48±0.03 ΔOD and 1.77±0.43 Δ log₁₀ for the S. dysgalactiae isolates (FIGS. 8 D, E & F and Table 5). NC5 can therefore be regarded a potent chimeric endolysin against S. uberis, S. agalactiae & dysgalactiae.

TABLE 5
Determination of the ΔOD₆₀₀, ΔLog₁₀ and lysis speed (i.e., (ΔOD₆₀₀/min)/μM) on mastitis
isolates of (sub)clinical infected dairy cows. Bacteria were challenged during 1 h with 0.3
μM of either NC2 or NC5. MALDI-TOF MS biotyping was performed to confirm the bacterial species
of the isolates. A score to each isolate is assigned indicating a non- (<1.70) to low (≥1.70)
or high (≥2.00) confidence level. Whole genome sequencing was performed to assign a known sequencing
type (ST) or global clonal complex (GCC) to the S. uberis isolates. These sequencing data confirmed
the bacterial species in addition to MALDI-TOF MS biotyping for S. uberis.

| | MALDI- | | NC2 | | | NC5 | | |
|---|---|---|---|---|---|---|---|---|
| | TOF Score | | | ΔLog₁₀ | (ΔOD₆₀₀/ | | ΔLog₁₀ | (ΔOD₆₀₀/ |
| Mastitis | (confidence | Genomic | | (CFU/ | min)/ | | (CFU/ | min)/ |
| isolate | level) | Characteristics | ΔOD₆₀₀ | mL) | μM | ΔOD₆₀₀ | mL) | μM |
| Clinical | 2.44 (High) | Unknown ST | 0.32 ± | 0.00 ± | 0.03 ± | 0.42 ± | 0.03 ± | 0.06 ± |
| S. uberis 1 | | and GCC | 0.00 | 0.48 | 0.00 | 0.01 | 0.26 | 0.00 |
| Clinical | 2.35 (High) | Unknown | 0.54 ± | 4.16 ± | 0.08 ± | 0.38 ± | 4.05 ± | 0.11 ± |
| S. uberis 2 | | ST, GCC 5 ° | 0.02 | 0.15 | 0.01 | 0.02 | 0.07 | 0.00 |
| Clinical | 2.31 (High) | Unknown | 0.72 ± | 1.66 ± | 0.22 ± | 0.62 ± | 1.82 ± | 0.44 ± |
| S. uberis 3 | | ST, GCC 5 ° | 0.01 | 0.17 | 0.02 | 0.01 | 0.18 | 0.01 |
| Clinical | 2.43 (High) | ST 815, | 0.30 ± | 0.01 ± | 0.04 ± | 0.63 ± | 0.00 ± | 0.05 ± |
| S. uberis 4 | | GCC 143 ° | 0.06 | 0.21 | 0.00 | 0.01 | 0.22 | 0.00 |
| Clinical | 2.33 (High) | ST 944, | 0.63 ± | 0.86 ± | 0.12 ± | 0.59 ± | 0.71 ± | 0.21 ± |
| S. uberis 5 | | GCC 5 | 0.01 | 0.11 | 0.01 | 0.01 | 0.13 | 0.00 |
| Subclinical | 2.42 (High) | ST 808, | 0.65 ± | 2.84 ± | 0.18 ± | 0.66 ± | 3.02 ± | 0.43 ± |
| S. uberis 1 | | GCC 143 | 0.00 | 0.12 | 0.01 | 0.01 | 0.37 | 0.01 |
| Subclinical | 2.47 (High) | Unknown ST | 0.59 ± | 1.02 ± | 0.06 ± | 0.65 ± | 1.13 ± | 0.11 ± |
| S. uberis 2 | | and GCC | 0.01 | 0.20 | 0.01 | 0.01 | 0.12 | 0.00 |
| Subclinical | 2.28 (High) | Unknown | 0.67 ± | 1.31 ± | 0.18 ± | 0.53 ± | 1.24 ± | 0.39 ± |
| S. uberis 3 | | ST, GCC 5 ° | 0.01 | 0.25 | 0.01 | 0.02 | 0.10 | 0.01 |
| Subclinical | 2.34 (High) | Unknown ST | 0.66 ± | 2.84 ± | 0.15 ± | 0.42 ± | 2.48 ± | 0.28 ± |
| S. uberis 4 | | and GCC | 0.00 | 0.37 | 0.01 | 0.00 | 0.34 | 0.00 |
| S. agalactiae 1 | 2.45 (High) | Not determined | | | | 0.42 ± | 1.49 ± | 0.15 ± |
| | | | | | | 0.00 | 0.12 | 0.01 |
| S. agalactiae 2 | 2.30 (High) | | | | | 0.39 ± | 1.50 ± | 0.08 ± |
| | | | | | | 0.02 | 0.02 | 0.00 |
| S. agalactiae 3 | 2.36 (High) | | | | | 0.41 ± | 0.00 ± | 0.12 ± |
| | | | | | | 0.00 | 0.20 | 0.00 |
| S. dysgalactiae 1 | 2.05 (High) | | | | | 0.48 ± | 1.77 ± | 0.25 ± |
| | | | | | | 0.03 | 0.43 | 0.01 |
| S. dysgalactiae 2 | 2.36 (High) | | | | | 0.66 ± | 0.15 ± | 0.30 ± |
| | | | | | | 0.03 | 0.07 | 0.00 |
| S. dysgalactiae 3 | 2.24 (High) | | | | | 0.51 ± | 1.11 ± | 0.25 |
| | | | | | | 0.01 | 0.17 | 0.01 |

° indicates the GCC was estimated based on sequence alignment against known GCCs from the pubMLST database.

1.5. Bactericidal Activity of NC5 Equals or Outperforms that of Natural Endolysins PlySs2 and PlySs9 Against S. uberis, S. agalactiae and S. dysgalactiae

### 1.5.1. Materials and Methods

Time killing assays were performed as previously described in 1.3.1.

### 1.5.2. Results

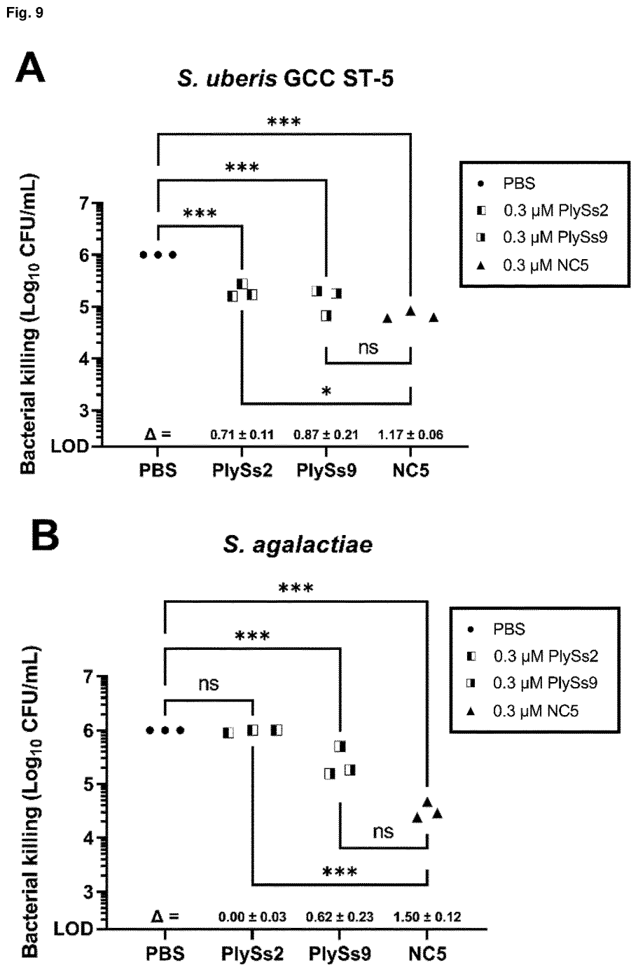

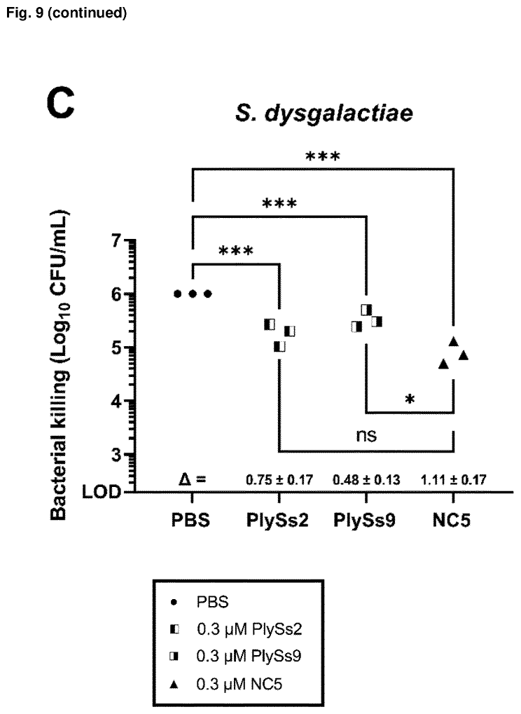

To finalize the screening assays, the bactericidal activity of NC5 was compared with that of the original wild type endolysins PlySs2 &-9 against one randomly selected mastitis-derived S. uberis GCC ST-5, S. agalactiae and S. dysgalactiae at 0.3 μM. This revealed that NC5 consistently showed the highest bacterial killing, with significant (p<0.001) logo reductions of 1.17±0.06, 1.50±0.12 and 1.11±0.17 against S. uberis GCC ST-5 and S. agalactiae & dysgalactiae, respectively (FIG. 9). PlySs2 &-9 also caused significant (p<0.001) log₁₀ reductions of 0.71±0.11 and 0.87±0.21 against S. uberis GCC ST-5, of which the PlySs2 log₁₀ reduction was found significantly (p<0.05) lower than that of NC5 (FIG. 9A). No significant (p >0.05) log₁₀ reduction was caused by PlySs2 against S. agalactiae in comparison with the PBS negative control, to which NC5 performed both significantly (p<0.001) better (FIG. 9B). PlySs9 also caused a significant (p<0.001) log₁₀ reduction of the selected S. agalactiae strain of 0.62±0.23, but this did not differ significantly (p >0.05) from NC5 (FIG. 9B). Concerning S. dysgalactiae, PlySs2 &-9 caused significant (p<0.001) log₁₀ reductions of 0.75±0.17 and 0.48±0.13, respectively (FIG. 9C). This latter observed logo reduction for PlySs2 did not differ significantly (p >0.05) from NC5, whereas the log₁₀ reduction for PlySs9 differed significantly (p<0.05) from NC5 (FIG. 9C). Taken together, NC5 significantly killed S. uberis GCC ST-5 (p<0.05) and S. agalactiae (p<0.001) better than PlySs2, killed S. dysgalactiae (p<0.05) better than PlySs9 and never showed inferior bactericidal activity compared to these wild type endolysins PlySs2 &-9 against these randomly selected mastitis-derived strains.

### Example 2—NC5 Eradicates S. uberis Biofilm

### Materials and Methods

Biofilms were grown by transferring mid-log phase bacteria (OD₆₀₀=0.6) grown in brain heart infusion (BHI) (Oxoid, Belgium) containing 0.25% α-D-glucose (ThermoFisher Scientific, USA) to a nunclon delta-treated, U-shaped-bottom 96-well microplate (ThermoFisher Scientific, USA). This whole was incubated for 21-24 h in a box containing a wet tissue. Subsequently, media and planktonic cells were removed and 1.5 UM of chimeric endolysin was allowed to eradicate the biofilms during 2 h30 at 37° C. on a shaker at 120 rpm. Biofilm characterization was performed by proteinase K (100 μg/mL in 100 mM NaCl and 20 mM Tris; pH 7.5) (ThermoFisher Scientific, USA), DNase I (100 μg/mL in 150 mM NaCl and 1 mM CaCl₂); pH 5.0) (Sigma-Aldrich, USA) or NaIO₄ (10 mM in 50 mM sodium acetate buffer; pH 4.5) (Sigma-Aldrich, USA) treatment for 1 h on a shaker at 120 rpm and 37° C. Biomass was fixated with 100% ethanol (ThermoFisher Scientific, USA) and stained with crystal violet (0.1% crystal violet (Sigma-Aldrich, USA), 5% methanol and isopropanol (ThermoFisher Scientific, USA) in PBS. Triple washing with PBS by inverting the plate was executed before solubilizing the stained biomass in 30% acetic acid (Chem-Lab, Belgium). OD_{590 nm} was measured using a CLARIOstar Plus plate reader (BMG Labtech, The Netherlands). Logarithmic reductions were determined by scraping biomass from the wells with a sterile tooth picker followed by resuspension in PBS, which was subsequently serially diluted and spotted on BHI-agar. CFU/mL were counted after overnight incubation at 37° C.

### Results

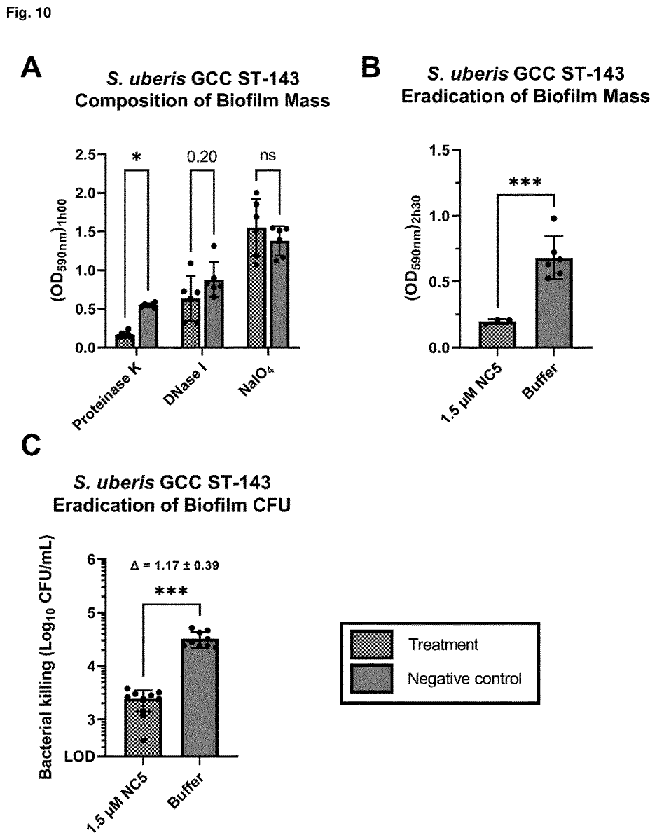

S. uberis GCC ST-143, which was found sensitive to NC5 (FIG. 8B), could reproducibly form a biofilm in vitro that was mainly composed of protein, as proteinase K treatment significantly (p<0.05) eradicated its biofilm mass (FIG. 10A). Upon challenging this biofilm with 1.5 μM NC5 during 2 h30, a significant (p<0.001) reduction of the biofilm mass corresponding to a significant (p<0.001) 1.17±0.39 Δ Log₁₀ reduction was observed (FIGS. 10B&C).

### Example 3-NC5 Tackles S. uberis Intracellularly

### Materials and Methods

### Cell Culture Conditions

MAC-T cells in passage 10-15 were grown in culture flasks at 37° C. and 5% CO₂ in Dulbecco's Modified Eagle's Medium (DMEM) (Gibco, USA) with the addition of 10% fetal bovine serum (FBS) (Gibco, USA), 0.5% insulin-transferrin-selenium (Gibco, USA) and 100 U/mL Penicillin-Streptomycin (P/S) (Gibco, USA). PS cells in passage 10-15 were grown in culture flasks at 37° C. and 5% CO₂ in Advanced DMEM Ham's F-12 (DMEM/F-12) (Gibco, USA) with the addition of 20 mM HEPES (Gibco, USA), 2 mM glutamine (Gibco, USA), 100 U/mL P/S, 1 μg/mL hydrocortisone 21-hemisuccinate (Sigma-Aldrich, USA), 10 ng/ml insulin-like growth factor-I (Peprotech, France), 5 ng/ml fibroblast growth factor (Peprotech, France) and epidermal growth factor (Sigma-Aldrich, USA). Cell cultures were routinely checked for Mycoplasma contamination using a Mycoplasma detection kit (InvivoGen, USA). After washing with Dulbecco's PBS (dPBS) (Gibco, USA), cells were harvested using 0.25% trypsin-ethylenediaminetetraacetic acid (EDTA) (Sigma-Aldrich, USA), which was inactivated once the cells were detached from the culture flask by adding an equal volume of 20% FBS in dPBS. Cells were centrifuged (200 g for 5 min), resuspended in cell culture medium, counted by means of a Bürker chamber and seeded (ratio 1:3 and 1:5 for MAC-T and PS, respectively).

### Intracellular Detection of NC5 by Western Blot

MAC-T cells were seeded in a 6-well plate at 300.000 cells/well and grown to 100% confluency. Next, cell culture medium was replaced by prewarmed NC5 in DMEM (buffer exchange performed at 4-8° C. by means of a Pierce™ Protein Concentrator PES 10 kDa MWCO) and allowed to incubate during 1 h at 37° C. and 5.0% CO₂. DMEM alone served as the negative control. Following incubation, the supernatant was aspirated and cells were washed 5× with prewarmed dPBS. Cells were then harvested, pelleted and washed as previously described, followed by lysis in ice-cold 0.1% triton X-100 (Sigma-Aldrich, USA) in dPBS. The cell lysates were denaturized and subjected to SDS-PAGE before blotting to a nitrocellulose membrane (Novolab, Belgium). The membrane was thereafter incubated during 1 h in dPBSM5 as blocking buffer. Subsequently, the primary antibody being mouse monoclonal IgG anti-HIS5 (Sigma-Aldrich, USA) was added to the blocking buffer at 1:2000 and allowed to incubate under the same conditions for another hour. The nitrocellulose membrane was then washed 3× with dPBST and incubated during 30 min with the secondary antibody being HRP conjugated goat monoclonal anti-mouse IgG (ImTec Diagnostics, Belgium) diluted 1:1000 in dPBST. For immunodetection, the nitrocellulose membrane was washed 3× with wash buffer and 1× with dH20 before incubation in the dark in 1-Step Ultra TMB-Blotting solution (ThermoFisher Scientific, USA). All incubation steps were performed at RT on a shaker. Regarding immunofluorescence, the fivefold washed MAC-T cells were fixated and permeabilized on the glass of the chamber slide by means of an inside stain kit (Miltenyi Biotec, The Netherlands). Primary antibody, i.e. rabbit monoclonal IgG anti-HIS6 (Abcam, The Netherlands), was administered to the fixated cells at 1:100 in Inside Perm solution and allowed to bind during 1 h. Next, cells were washed 3× with Inside Perm solution and treated for 30 min. with Inside Perm solution containing 2 μg/mL Hoescht 33342 (ThermoFisher Scientific, USA), 1:1000 Phalloidin-iFluor 594 (Abcam, The Netherlands) and 1:200 AlexaFluor 488-labeled Goat monoclonal IgG anti-rabbit (Abcam, The Netherlands). Finally, cells were triple washed with Inside Perm solution and mounted with a cover slide using anti-fading glycerol mounting medium with DABCO.

### Intracellular Killing Assay

Cells were seeded in a 24-well plate at 50.000 cells/well with the addition of 25 μg/mL calf skin collagen type I (dissolved in 0.1 M acetic acid) (Sigma-Aldrich, USA) and grown to 100% confluency. Next, medium was removed and cells were washed 3× with dPBS to remove residual P/S. An overnight bacterial culture was washed with dPBS, dissolved in an equal volume of prewarmed cell culture media without the addition of P/S and 0.5 mL thereof was incubated with the cells in every well during 3 h at 37° C. to allow intracellular invasion. Afterwards, this suspension was removed, serially diluted and plated as previously described to determine the amount of extracellular bacteria. Cells were again washed 3× with dPBS and 0.5 mL of prewarmed cell culture media was added with the addition of 200 μg/ml gentamycin (Sigma-Aldrich, USA) and incubated during 3 h at 37° C. to kill remaining extracellular and adhered bacteria. Next, this suspension was likewise removed and plated to check if killing of the extracellular and adhered bacteria was successful. Cells were again 3× washed with dPBS and 2.5 UM of prewarmed NC5 in cell culture media without P/S (buffer exchange performed at 4-8° C. by means of a Pierce™ Protein Concentrator PES 10 kDa MWCO) was added during 1 h to eradicate the intracellular bacteria. Subsequently, cells were 3× washed with dPBS and detached from the wells by trypsinization as previously described. Their viability was checked at this point by trypan blue staining (Sigma-Aldrich, USA). Cells were pelleted and resuspended in dPBS containing 0.1% triton x-100 to lyse the eukaryotic cells and release the intracellular bacteria. This whole was subsequently serially diluted, plated on BHI-agar and incubated overnight at 37° C. in order to determine the amount of intracellular bacteria the following day.

### Results

### NC5 is Present Inside Bovine Mammary Epithelial Cells

To show that NC5 can enter bovine mammary epithelial cells, lysate from MAC-T cells, which were incubated with 2.5 μM NC5, was analyzed by SDS-PAGE and western blot after washing away the extracellular protein. This showed that NC5 neither disintegrated nor precipitated after incubation with MAC-T cells, as was analyzed by SDS-PAGE in the supernatant fraction. The latter also showed that all extracellular protein was washed away before the cells were harvested and immunodetection was performed by western blot on MAC-T lysates. A band at the expected size of NC5 was present in the lysate of treated MAC-T cells, which was absent in the lysate of untreated cells. To further support this key finding that indicates NC5 is present intracellularly, cells that were likewise treated with 2.5 μM NC5 and were visualized by confocal microscopy. This latter technique clearly revealed the intracellular presence of NC5 in the MAC-T cells (microscopy image not shown). By means of control, autofluorescence was checked and untreated MAC-T were likewise stained and visualized. As expected, no autofluorescence was present and no fluorescent signal corresponding to NC5 could be retrieved from the untreated cells. Taken together, these data show that NC5 is able to enter MAC-T.

NC5 Eradicates S. uberis Intracellularly

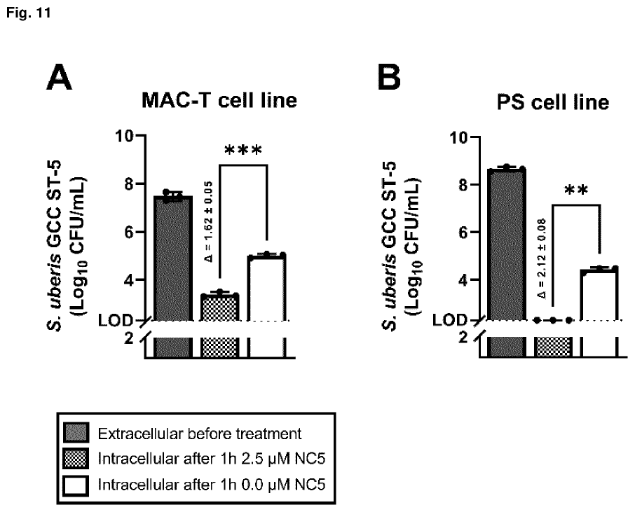

MAC-T and PS bovine mammary epithelial cell lines (BoMEC) were challenged with a S. uberis GCC ST-5 isolate that was found sensitive to NC5 and showed predetermined intracellular invasion (i.e., clinical S. uberis 5 in Table 5). After 3 h co-incubation of S. uberis with both BoMEC, 7.46±0.14 and 8.66±0.08 log 10 were extracellularly retrieved from MAC-T and PS cells, respectively, corresponding to a multiplicity of infection (MOI) of approximately 250 and 500 (FIG. 11). After gentamycin treatment to kill the remaining, extracellular S. uberis, infected MAC-T and PS were challenged during 1 h with 2.5 μM NC5 in cell culture medium. The same medium without the addition of NC5 served as the negative control. From this negative control 5.00±0.06 and 4.42±0.08 log 10 were respectively retrieved from the infected MAC-T and PS cells (FIG. 11). In comparison with this negative control, both BoMEC showed a reduction of the intracellular S. uberis fraction after NC5 treatment. More specifically, a significant (p<0.001) reduction of 1.62±0.05 Δ Log₁₀ was observed for the infected MAC-T, whereas this was at least 2.12±0.08 Δ Log₁₀ for the infected PS as the remaining S. uberis all were below or equal to the detection limit of 200 CFU/mL (FIG. 11). Together, these data suggests that NC5 is able to eradicate S. uberis intracellularly in BoMEC.

### Example 4—NC5 Potentiates Cloxacillin Treatment Against S. uberis in Mastitic Raw Cow's Milk

### Materials and Methods

Raw cow's milk from S. uberis infected dairy cows was supplied by the milk control center of Flanders (MCC Vlaanderen). Upon arrival, the milk was serially diluted and plated on BHI agar to determine the bacterial load. Only milk with >10⁵ log₁₀ CFU/mL S. uberis was included as a selection criterium. If needed, the raw milk was incubated during 8 h at 37° C. to artificially increase the initial bacterial load. Next, triplicates of 160 μL mastitic milk were supplemented in a 96 microtiter plate with 40 μL of either dPBS, 2.5 μM NC5 in dPBS, 250 μg/mL cloxacillin sodium in dPBS (Sigma-Aldrich, USA) or a combination of the two latter (i.e., the combination therapy). This resulted in final concentrations of 0.5 μM (i.e., ±2×MIC) and 50 μg/mL (i.e., ±10×MIC) of NC5 and cloxacillin sodium in 4:5 diluted mastitic milk, respectively. Upon incubation at 37° C., every 2 h, 20 μL was transferred from each well to 180 μL dPBS, serially diluted and plated on BHI agar. Plates were incubated overnight and CFU/mL were calculated the next day. Undiluted, mastitic milk was always pipetted with ClipTip Pipette Tips (Westburg, The Netherlands) due to high viscosity. Mastitis isolates were confirmed as S. uberis by MALDI-TOF MS biotyping.

### Results

Mastitic raw cow's milk from 4 cows with a confirmed S. uberis infection was challenged during 8 h with either 0.5 μM NC5, 50 μg/mL cloxacillin, a combination of both (i.e., the combination therapy) or PBS as a negative control (FIG. 12). Cloxacillin is a penicillin derivative frequently used to intramammarily treat streptococcal mastitis in cows. Treatment with 0.5 μM NC5 alone caused a log₁₀ reduction in all mastitic milk samples after 8 h, but this was only significant (p<0.05) in 2 out of 4 samples compared to the PBS control (i.e., the mastitic milk from cow 2 & 4; FIG. 12). The log₁₀ reductions caused by 0.5 μM NC5 were inferior compared to that caused by 50 μg/mL cloxacillin, except for the mastitic milk from cow 2 at ≤6 h (FIG. 12). The cloxacillin and combination therapy both caused significant (p<0.05) reductions in all 4 mastitic milk samples at 8 h that attained the detection limit in 2 out of 4 samples (i.e., the mastitic milk from cow 2 & 4; FIG. 12). An important finding was that all mastitic milk samples showed a faster decrease of the S. uberis load if cloxacillin treatment was combined with 0.5 μM NC5 (FIG. 12), i.e., between 2 to 8 h and 0 to 6 h in the mastitic milk of cow 1 & 2 and 4, respectively. In the mastitic milk of cow 3, the combination therapy diverged from cloxacillin at 4 h and kept declining at least till 8 h (FIG. 12). The log₁₀ differences caused by the combination therapy differed significantly (p<0.05) from the 50 μg/mL cloxacillin alone group during at least 2 h in the mastitic milk samples of 3 out of 4 cows (i.e., cow 2, 3 and 4; FIG. 12). The same observation was made in the mastitic milk sample of cow 1, but here the log₁₀ differences were non-significant (p >0.05). Taken together, these data suggest that the chimeric endolysins of the invention such as NC5 act additively or synergistically with antibiotics such as cloxacillin in mastitic raw cow's milk and thus forms an adjunct to the standard point-of-care for the treatment of mastitis, in particular of S. uberis mastitis.

### Example 5—NC5 Improves Early Cloxacillin Treatment in a Mouse Model of S. uberis Mastitis

### Materials and Methods

Optimization of the Mouse Model for Bovine S. uberis Mastitis

All experimental procedures on mice were executed at the Faculty of Veterinary Medicine of Ghent University (Merelbeke, Belgium). Breeding pairs of female and male CD-1 mice (Envigo, The Netherlands) were allowed to mate during two weeks, after which the dams gave birth approximately seven days later. Twelve days postpartum, the lactating dams were intraductally inoculated with a blunted 32 gauge pediatric needle in the fourth inguinal mammary gland pair after properly disinfecting the teat. These intraductal inoculations were always performed under general gas anesthesia, using a mixture of medical oxygen and isoflurane at 2.5-3.0% for induction and 1.5-2.0% for maintenance. The long-acting analgesic buprenorphine was administered at 10 μg/kg intraperitoneally for post-surgical pain relief. An inoculum dose of approximately 103 colony forming units (CFU) of a clinical bovine mastitis S. uberis isolate in 100 μl dPBS was intraductally inoculated at 1 h post-weaning and mice were grouped ad random at 12 h post-infection (p.i.) and placed under gas anesthesia a second time to administer intramammary treatment. This treatment was administered in a volume of 150 μl and consisted of: (i) a combination therapy of 30.0 μg of cloxacillin sodium in dPBS and either a low or high dose of endolysin NC5 (i.e., 23.5 and 235.0 μg with number of mice n=6 and 10, respectively), (ii) 30.0 μg cloxacillin sodium in dPBS as antibiotic stand-alone therapy (n=11), or (iii) dPBS as the positive control for mastitis lesions or placebo treatment (n=4). The treated mice were sedated 16 h p.i. (i.e., 4 h post-treatment). The therapeutics were diluted in dPBS to their desired concentration, mixed by pipetting and kept on melting ice until they were administered intraductally within 1 h after preparation. Inoculum preparation was done by growing S. uberis to stationary phase in brain heart infusion (BHI), after which cells were washed with dPBS and diluted in dPBS to the desired concentration based on OD₆₀₀ measurements.

### Murine Mammary Gland Collection and Bacterial Load Determination

Mice received a cocktail of 100 mg/kg ketamine and 10 mg/kg xylazine and were subsequently euthanized by cervical dislocation to harvest the mammary glands. To quantify the bacterial loads, the isolated mammary glands were homogenized using a TissueRuptor and 1:10 serially diluted in dPBS followed by plating on BHI with the addition of 15.0 g/L agar. To compensate for the different weight of the harvested tissues, mammary glands were initially weighed and bacterial load was expressed as log₁₀ (CFU/g tissue). Harvested mammary glands were always kept cold on melting ice.

### Inflammatory Protein Profiling in Murine Mammary Gland Lysates

The host response to the experimental mammary gland infection and subsequent therapy was investigated by determining the levels of inflammatory cytokines and chemokines in mammary gland lysates, obtained by mixing the homogenates with 300 μL caspase lysis buffer (CLB) containing protease inhibitors, and subsequent centrifugation (12,000 g; 4° C.; 20 min). Protein concentration of these lysates was determined using the Bradford protein assay. Standardization of the mammary gland lysates was done by dilution in CLB to assure equal protein concentrations. Levels of the selected inflammatory mediators TNF-α, MCP-1, M-& G-CSF as well as IL-1α, -1β, -6, & -8, were determined on mammary gland lysates using a ProcartaPlex Immunoassay. Quantification of CHI3L1 and LCN2 was performed by ELISA.

### Histology and Immunohistochemistry on Murine Mammary Gland Sections

Harvested mammary glands were fixed for 24 h in 3.5% buffered formaldehyde for subsequent embedding in paraffin. Histology required deparaffinization and rehydration of 5 μm sections followed by short (5 min) incubation in H&E staining buffers. Neutrophils and macrophages were stained by immunohistochemistry (IHC) for the neutrophil marker Ly6G (anti-Ly6G) and macrophage marker Iba-1 (anti-lba-1) on paraffin sections. Deparaffinized and hydrated tissue slides were incubated in citrate buffer (i.e., 10 mM tri-sodium citrate, 0.05% Tween-20, pH 6.0) in a decloacking chamber under pressure at 95° C. for 30 min. Following endogenous peroxidase blocking (3% H₂O₂ in methanol for 10 min at 22° C.), sections were incubated with primary rat anti-mouse Ly6G antibody (diluted 1:1000 in antibody diluent) or primary rabbit anti-mouse Iba-1 (diluted 1:2000) for 1 h at 22° C. Rat-on-mouse or rabbit-on-rodent HRP-Polymer served as secondary antibody on the tissue sections for 30 min at 22° C. Detection of the staining was performed by applying 3,3′-diaminobenzidine on the tissue sections for 10 min. For microscopic evaluation, tissue sections were rehydrated and mounted with a cover glass. Throughout the staining procedure, slides were kept in a humidified box and washed between each incubation step with Tris-buffered saline. ImageJ was used to quantify positive staining (i.e., color deconvolution and automatic counting of % area), as described previously.

### Results

It was evaluated if NC5 in either a low or a high dose (i.e., 23.5 and 235.0 μg, respectively), combined with cloxacillin, reduces local inflammation by evaluating the hallmarks of mastitis (i.e. clinical symptoms, bacterial load, MIP-2 (i.e. IL-8) levels and microscopic influx of neutrophils) and quantifying (pro) inflammatory mediator levels, in comparison to cloxacillin as stand-alone antibiotic therapy and to placebo treatment.

Mice did not display clinical symptoms or macroscopic abnormalities of the mammary glands in all groups, except for one mouse in the high dose combination therapy group in which swelling, increased vascularization and redness were observed in both glands. The bacterial load in the mammary glands at 16 h p.i. decreased in both combination therapy groups compared to cloxacillin as stand-alone therapy, with the highest decrease observed for the high endolysin dose (FIG. 13A). More specifically, stand-alone antibiotic treatment caused—on average—a 55-fold decrease of the bacterial load compared to the placebo group (p=0.43), while 225- to 500-fold decreases (p=0.30 and 0.14) were seen upon addition of the low and high endolysin dose, respectively. Unexpectedly, it was observed that the antibiotic stand-alone and both combination therapy groups showed a high variability indicative for two types of responders, i.e. mice that already showed a response after the 4 h treatment (n=15) and mice that showed no clear response at that early time point yet (n=10). If these presumed slow responding mice were excluded, based on the criterium that their values were within the mean±2×SEM of the placebo group (i.e., 8.04±1.42 log₁₀ (CFU/g tissue)), significance could be demonstrated for all therapy groups in comparison with the placebo group. More specifically, an average decrease of 630-; 4,900- and 13,000-fold (p=0.007, 0.002 and p<0.001) was observed in CFU numbers for the antibiotic stand-alone, the low and high dose combination therapy groups in these presumed fast responders, respectively (FIG. 13B). Upon histopathological evaluation of these presumed fast responder mice, the mammary glands of the different treatment groups showed an improvement of the microscopic mastitic lesions (microscopy images not shown). More specifically, whereas a variable influx of PMN was observed in the placebo group, this influx was observed to a lesser extent in the group that received the antibiotic stand-alone therapy and it continued to decrease even further upon addition of both the low and high dose of the endolysin to cloxacillin. This was corroborated upon quantification of PMN by Ly6G-positive staining, which revealed a 1.8-, 2.0- and 5.7-fold reduced (p=0.95, 0.95 and 0.78) neutrophil influx in the antibiotic stand-alone as well as the low and high dose combination therapy groups vs. the placebo group, respectively (FIG. 13C). Complementary inflammatory protein profiling of this main in vivo proof-of-concept experiment revealed an overall dose-dependent reduction of pro-inflammatory mediators caused by the supplementation of the endolysin NC5 to cloxacillin (FIG. 14). Interestingly, this effect was observed regardless of the exclusion of the slow-responding mice-except for G-CSF and LCN2-indicating that even mice with a lack of reduction in bacterial load still exhibited an overall immunological response. Supporting the overall decrease in PMN influx as observed microscopically, the concentration of the neutrophil chemokine MIP-2 showed a dose-dependent reduction when NC5 was added to cloxacillin (FIG. 14A). More specifically, a significant 3.3- and 5.0-fold decrease (p=0.01 and 0.002) for MIP-2 was observed comparing the low and high combination therapy groups with the placebo group, respectively. Only a 1.6-fold decrease (p=0.14) was observed for the cloxacillin treatment compared to the placebo group. Consequently, there was a significant (p=0.027) difference in MIP-2 levels between the antibiotic stand-alone and high dose combination therapy groups (FIG. 14A). Consistent with these findings for MIP-2, a similar trend was observed for the neutrophil-maturating cytokine G-CSF and the neutrophil-associated antimicrobial iron-chelating protein LCN2, albeit only for the presumed fast responding mice (FIG. 14B-C). More specifically, the antibiotic stand-alone, low and high combination therapy groups showed a 2.0-, 1.9- and a 93.7-fold G-CSF decrease (p=0.34, 0.34 and 0.029, respectively) in comparison with the placebo group, respectively. For LCN2 this reduction was 1.2-, 1.3- and 1.6-fold (p=0.93, 0.93 and 0.71, respectively). Notably, addition of a high dose of NC5 did not further decrease the G-CSF and LCN2 levels if the presumed slow responding mice were taken into account. Indeed, local concentrations of LCN2 were even trended upward slightly 1.1-fold (p=0.82) in the high vs. low dose combination therapy groups, respectively.

The local immune profile was further complemented by including the general pro-inflammatory mediators IL-1α, -1β, -6 and TNF-α (FIG. 14D-G). Overall, these concentrations were again reduced dose-dependently by addition of endolysin NC5 to cloxacillin. More specifically, IL-1α, -1β, -6 and TNF-α concentrations decreased 2.4-, 2.4-, 4.1- and 2.4-fold (p=0.10, 0.05, 0.003 and 0.007) in the group that received the high dose combination therapy in comparison with the placebo group, whereas these decreases were 1.2-, 1.4-, 2.2- and 1.8-fold (p=0.89, 0.46, 0.06 and 0.08) for the low dose combination therapy group, and 1.2-, 1.3-, 2.0- and 1.4-fold (p=0.89, 0.46, 0.06 and 0.51) for the cloxacillin stand-alone group, respectively. The two macrophage-associated inflammatory mediators, monocyte chemoattractant protein (MCP)-1 and macrophage-colony stimulating factor (M-CSF) showed similar dose-dependent reductions after addition of the endolysin to cloxacillin (FIG. 14H-I). More specifically, levels of MCP-1 and M-CSF trended downward slightly 1.5-, 2.1- and were significantly reduced 8.0-fold (p=0.39, 0.25 and 0.02) for MCP-1, as well as 1.1, 1.4 and 1.9-fold (p=0.51, 0.46 and 0.04) for M-CSF, respectively for the antibiotic stand-alone, low and high dose combination therapy groups in comparison with the placebo group. Similar to MIP-2, there was a significant difference in M-CSF levels between the antibiotic stand-alone and the high dose combination therapy groups (p=0.036). In addition, to evaluate the influx of macrophages in the treated murine mammary glands, IHC for Iba-1 was performed. This staining showed the moderate presence of mammary ductal macrophages that physiologically reside between the luminal and basal epithelial cells of the lactating mammary gland, but no differences in the number of macrophage between the treatment groups was observed (microscopy images not shown). Finally, the lesser-known but complementary innate immunity marker CHI3L1 was also determined (FIG. 14J). Supplementation of NC5 to cloxacillin again resulted in a dose-dependent trending downward of the local CHI3L1 concentrations, 1.4-, 2.0- and 3.6-fold (p=0.70, 0.43 and 0.11) for the antibiotic stand-alone, low and high dose combination therapy groups in comparison with the placebo group, respectively.

### REFERENCES

Schmelcher M, Powell A M, Camp M J, Pohl C S, Donovan D M. Synergistic streptococcal phage ASA2 and B30 endolysins kill streptococci in cow milk and in a mouse model of mastitis. Appl Microbiol Biotechnol. 2015 October; 99 (20): 8475-86. doi: 10.1007/s00253-015-6579-0. Epub 2015 Apr. 21. PMID: 25895090; PMCID: PMC4573782.
Liu G, Zhang S, Gao T, Mao Z, Shen Y, Pan Z, Guo C, Yu Y, Yao H. Identification of a novel broad-spectrum endolysin, Ply0643, with high antibacterial activity in mouse models of streptococcal bacteriaemia and mastitis. Res Vet Sci. 2022 March; 143:41-49. doi: 10.1016/j.rvsc.2021.12.014. Epub 2021 Dec. 23. PMID: 34973538.
Nelson D, Schuch R, Chahales P, Zhu S, Fischetti V A. PlyC: a multimeric bacteriophage lysin. Proc Natl Acad Sci USA. 2006 Jul. 11; 103 (28): 10765-70. doi: 10.1073/pnas.0604521103. Epub 2006 Jul. 3. PMID: 16818874; PMCID: PMC1487170.
Yang H, Linden S B, Wang J, Yu J, Nelson D C, Wei H. A chimeolysin with extended-spectrum streptococcal host range found by an induced lysis-based rapid screening method. Sci Rep. 2015 Nov. 26; 5:17257. doi: 10.1038/srep17257. PMID: 26607832; PMCID: PMC4660466.
N. Vander Elst, S. B. Linden, R. Lavigne, E. Meyer, Y. Briers, and D. C. Nelson, “Characterization of the bacteriophage-derived endolysins plyss2 and plyss9 with in vitro lytic activity against bovine mastitis streptococcus uberis,” Antibiotics, vol. 9, no. 9, pp. 1-14, 2020, doi: 10.3390/antibiotics9090621.
Gutiérrez D, Garrido V, Fernández L, Portilla S, Rodríguez A, Grilló M J, García P. Phage Lytic Protein LysRODI Prevents Staphylococcal Mastitis in Mice. Front Microbiol. 2020 Jan. 23; 11:7. doi: 10.3389/fmicb.2020.00007. PMID: 32038593; PMCID: PMC6989612.
Fan J, Zeng Z, Mai K, Yang Y, Feng J, Bai Y, Sun B, Xie Q, Tong Y, Ma J. Preliminary treatment of bovine mastitis caused by Staphylococcus aureus, with trx-SA1, recombinant endolysin of S. aureus bacteriophage IME-SA1. Vet Microbiol. 2016 Aug. 15; 191:65-71. doi: 10.1016/j.vetmic.2016.06.001. Epub 2016 Jun. 6. PMID: 27374909.
Donovan D M, Lardeo M, Foster-Frey J. Lysis of staphylococcal mastitis pathogens by bacteriophage phi11 endolysin. FEMS Microbiol Lett. 2006 December; 265 (1): 133-9. doi: 10.1111/j.1574-6968.2006.00483.x. Epub 2006 Oct. 19. PMID: 17054440.
Obeso J M, Martínez B, Rodríguez A, García P. Lytic activity of the recombinant staphylococcal bacteriophage PhiH5 endolysin active against Staphylococcus aureus in milk. Int J Food Microbiol. 2008 Dec. 10; 128 (2): 212-8. doi: 10.1016/j.ijfoodmicro.2008.08.010. Epub 2008 Aug. 26. PMID: 18809219.
Son B, Kong M, Lee Y, Ryu S. Development of a Novel Chimeric Endolysin, Lys109 With Enhanced Lytic Activity Against Staphylococcus aureus. Front Microbiol. 2021 Jan. 15; 11:615887. doi: 10.3389/fmicb.2020.615887. PMID: 33519773; PMCID: PMC7843465.
H. Gerstmans et al., “A VersaTile-driven platform for rapid hit-to-lead development of engineered lysins,” Sci. Adv., vol. 6, no. 23, pp. 1-12, 2020, doi: 10.1126/sciadv.aaz1136.
Chen H, Mao R, Teng D, Wang X, Hao Y, Feng X, Wang J. Design and pharmacodynamics of recombinant NZ2114 histidine mutants with improved activity against methicillin-resistant Staphylococcus aureus. AMB Express. 2017 December; 7 (1): 46

1. A fusion protein, comprising
a cell penetrating peptide (CPP);
at least one enzymatic activity domain (EAD); and
at least one cell wall binding domain (CBD) comprising an amino acid sequence selected from SEQ ID NO: [1], SEQ ID NO: [2], or a sequence having at least 85% identity thereto.

2. The fusion protein according to claim 1, wherein said at least one EAD comprises an amino acid sequence as set forth in SEQ ID NO: [3], or a sequence having at least 85% identity thereto.

3. The fusion protein according to claim 2, wherein said fusion protein comprises at least one further EAD comprising an amino acid sequence as set forth in SEQ ID NO: [4], or a sequence having at least 85% identity thereto.

4. The fusion protein according to any one of claims 1 to 3, wherein said at least one CBD comprises an amino acid sequence as set forth in SEQ ID NO: [1], or a sequence having at least 85% identity thereto.

5. The fusion protein according to any one of claims 3 to 4, wherein said fusion protein comprises the peptide and domains in the order CPP-EAD-CBD-EAD from the N-terminal to the C-terminal position.

6. The fusion protein according to any one of claims 1 to 5, comprising an amino acid sequence as set forth in SEQ ID NO: [17], or a sequence having at least 85% identity thereto.

7. The fusion protein according to any one of claims 1 to 5, comprising an amino acid sequence as set forth in SEQ ID NO: [20, 15], or a sequence having at least 85% identity thereto.

8. The fusion protein according to any one of claims 1 to 5, comprising an amino acid sequence as set forth in SEQ ID NO: [14], or a sequence having at least 85% identity thereto.

9. An isolated nucleic acid encoding the fusion protein according to any one of claims 1 to 8.

10. The isolated nucleic acid according to claim 9, wherein said nucleic acid comprises a nucleic acid sequence that has at least 65% identity as set forth in SEQ ID NO: [10].

11. A vector comprising the nucleic acid of claims 9 or 10.

12. A host cell expressing the fusion protein according to any one of claims 1 to 8, or comprising the nucleic acid of claim 9 or 10, or the vector of claim 11.

13. A pharmaceutical composition or combination comprising the fusion protein of any one of claims 1 to 8, the nucleic acid of claim 9 or 10, the vector of claim 11, or the host cell of claim 12, and a pharmaceutically acceptable excipient.

14. The pharmaceutical composition or combination according to claim 13, further comprising at least one antibiotic, in particular a beta-lactam antibiotic, more in particular a penicillin, even more in particular cloxacillin.

15. The fusion protein of any one of claims 1 to 8, the nucleic acid of claim 9 or 10, the vector of claim 11, or the host cell of claim 12, the pharmaceutical composition or combination according to claim 13 or 14, for use in medicine, in particular for use in veterinary medicine, more in particular for use in the treatment and/or prevention of an infection caused by Streptococcus uberis, Streptococcus dysgalactiae, Streptococcus agalactiae and/or Staphylococcus aureus in a subject, in particular for the treatment and/or prevention of mastitis, in particular ruminant mastitis, more in particular bovine mastitis.
